(19) United States
(12) Patent Application Publication
(10) Pub. No.: US 2024/0301484 A1
(43) Pub. Date: Sep. 12, 2024

(54) ENZYME METHOD

(71) Applicant: Oxford Nanopore Technologies PLC, Oxford (GB)

(72) Inventors: Ruth Moysey, Oxford (GB); Andrew John Heron, Oxford (GB)

(73) Assignee: Oxford Nanopore Technologies PLC, Oxford (GB)

(21) Appl. No.: 18/673,074

(22) Filed: May 23, 2024

Related U.S. Application Data
(63) Continuation of application No. 17/829,200, filed on May 31, 2022.
Application No. 17/829,200 is a continuation of application No. 16/902,301, filed on Jun. 16, 2020, now Pat. No. 11,634,763.
Application No. 16/902,301 is a continuation of application No. 15/674,653, filed on Aug. 11, 2017, now Pat. No. 10,724,087.
Application No. 15/674,653 is a continuation of application No. 14/351,038, filed on Apr. 10, 2014, now Pat. No. 9,758,823.
(60) Provisional application No. 61/599,244, filed on Feb. 15, 2012.
(60) Provisional application No. 61/549,998, filed on Oct. 21, 2011.

(51) Int. Cl. C12Q 1/6869

(52) U.S. Cl. CPC C12Q 1/6869; C12Q 2521/513; C12Q 2565/631

(57) ABSTRACT

The invention relates to a new method of characterizing a target polynucleotide. The method uses a pore and a Hel308 helicase or amolecular motor which is capable of binding to the target polynucleotide at an internal nucleotide. The helicase or molecular motor controls the movement of the target polynucleotide through the pore.

### FIELD OF THE INVENTION

The invention relates to a new method of characterising a target polynucleotide. The method uses a pore and a Hel308 helicase or a molecular motor which is capable of binding to the target polynucleotide at an internal nucleotide. The helicase or molecular motor controls the movement of the target polynucleotide through the pore.

### BACKGROUND OF THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the “Strand Sequencing” method, a single polynucleotide strand is passed through the pore and the identity of the nucleotides are derived. Strand Sequencing can involve the use of a nucleotide handling protein to control the movement of the polynucleotide through the pore.

### SUMMARY OF THE INVENTION

The inventors have demonstrated that a Hel308 helicase can control the movement of a polynucleotide through a pore especially when a potential, such as a voltage, is applied. The helicase is capable of moving a target polynucleotide in a controlled and stepwise fashion against or with the field resulting from the applied voltage. Surprisingly, the helicase is capable of functioning at a high salt concentration which is advantageous for characterising the polynucleotide and, in particular, for determining its sequence using Strand Sequencing. This is discussed in more detail below.

Accordingly, the invention provides a method of characterising a target polynucleotide, comprising:
(a) contacting the target polynucleotide with a transmembrane pore and a Hel308 helicase such that the helicase controls the movement of the target polynucleotide through the pore and nucleotides in the target polynucleotide interact with the pore; and
(b) measuring one or more characteristics of the target polynucleotide during one or more interactions and thereby characterising the target polynucleotide.

The invention also provides:
a method of forming a sensor for characterising a target polynucleotide, comprising forming a complex between a pore and a Hel308 helicase and thereby forming a sensor for characterising the target polynucleotide;
use of a Hel308 helicase to control the movement of a target polynucleotide through a pore;
a kit for characterising a target polynucleotide comprising (a) a pore and (b) a Hel308 helicase; and
an analysis apparatus for characterising target polynucleotides in a sample, comprising a plurality of pores and a plurality of a Hel308 helicase.

The inventors have also demonstrated that a molecular motor which is capable of binding to a target polynucleotide at an internal nucleotide can control the movement of the polynucleotide through a pore especially when a potential, such as a voltage, is applied. The motor is capable of moving the target polynucleotide in a controlled and stepwise fashion against or with the field resulting from the applied voltage. Surprisingly, when the motor is used in the method of the invention it is possible to control the movement of an entire strand of target polynucleotide through a nanopore. This is advantageous for characterising the polynucleotide and, in particular, for determining its sequence using Strand Sequencing.

Hence, the invention also provides a method of characterising a target polynucleotide, comprising:
(a) contacting the target polynucleotide with a transmembrane pore and a molecular motor which is capable of binding to the target polynucleotide at an internal nucleotide such that the molecular motor controls the movement of the target polynucleotide through the pore and nucleotides in the target polynucleotide interact with the pore; and
(b) measuring one or more characteristics of the target polynucleotide during one or more interactions and thereby characterising the target polynucleotide.

### DESCRIPTION OF THE FIGURES

FIG. 1. a) Example schematic of use of a helicase to control DNA movement through a nanopore. 1) A ssDNA substrate (a) with an annealed primer (b) containing a cholesterol-tag (c) is added to the cis side of the bilayer (d). The cholesterol tag binds to the bilayer, enriching the substrate at the bilayer surface. 2) Helicase (e) added to the cis compartment binds to the DNA. In the presence of divalent metal ions and NTP substrate, the helicase moves along the DNA. 3) Under an applied voltage, the DNA substrate is captured by the nanopore (f) via the leader section on the DNA. The DNA is pulled through the pore under the force of the applied potential until a helicase, bound to the DNA, contacts the top of the pore, preventing further uncontrolled DNA translocation. During this process dsDNA sections (such as the primer) are removed. The helicase movement along the DNA in a 3′ to 5′ direction pulls the threaded DNA out of the pore against the applied field. 4) The helicase pulls the DNA out of the nanopore, feeding it back to the cis compartment. The last section of DNA to pass through the nanopore is the 5′-leader. 5) When the helicase moves the DNA out of the nanopore it is lost back to the cis compartment. b) The DNA substrate design used in the Example (a=400 mer strand of DNA with a 50T leader (b), c=primer, d=cholesterol-tag).

FIG. 2. Helicase is able to move DNA through a nanopore in a controlled fashion, producing stepwise changes in current as the DNA moves through the nanopore. Example helicase-DNA events (indicated by small arrows in the top section) (180 mV, 400 mM KCl, Hepes pH 8.0, 0.15 nM 400 mer DNA, 100 nM Hel308 Mbu, 1 mM DTT, 1 mM ATP, 1 mM MgCl₂). Top) Section of current (y-axis, pA) vs. time (x-axis, s) acquisition of Hel308 400 mer DNA events. The open-pore current is ˜180 pA (labelled A). DNA is captured by the nanopore under the force of the applied potential (+180 mV). DNA with enzyme attached results in a long block (at ˜60 pA in this condition) that shows stepwise changes in current as the enzyme moves the DNA through the pore. Middle) The middle section is an enlargement of one of the DNA events (1), showing DNA-enzyme capture, stepwise current changes as the DNA is pulled through the pore, and ending in a characteristic long polyT level before exiting the nanopore. Bottom) enlargement of the stepwise changes in current as DNA is moved through the nanopore.

FIG. 3. Helicase controlled DNA movement resulting in a consistent pattern of current transitions as DNA is passed through the nanopore (y-axis=current (pA), x-axis=time (s) for FIGS. 3a and 3b). Examples of the last ˜80 current transitions from four typical DNA events that end in the polyT level. The four examples (two in 3a and two in 3b) illustrate that a consistent pattern of current transitions are observed.

FIG. 4. Increased salt concentration increases pore current and gives a larger DNA discrimination range (range=minimum current to maximum current across the DNA current transitions). Example helicase-DNA events (y-axis=current (pA), x-axis=time (s) for FIGS. 4a-c, 180 mV, Hepes pH 8.0, 0.15 nM 400 mer DNA SEQ ID NOs: 59 and 60, 100 nM Hel308 Mbu, 1 mM DTT, 1 mM ATP, 1 mM MgCl₂) at 400 mM, 1 M, and 2 M KCl are shown in FIGS. 4 and 4c. Top traces show a full event that ends in the polyT level (with I-open indicated by A), and lower traces show a zoom section of the last 10 seconds of each event with a constant y-axis current scale of 150 pA. Increasing the salt concentration from 400 mM KCl to 2M KCl leads to a ˜350% increase in the open-pore current (I-open from ˜180 pA to ˜850 pA), and a ˜200% increase in discrimination range (˜25 pA to ˜75 pA) FIG. 4d is a plot of DNA discrimination range as a function of salt concentration (y-axis=range (pA), x-axis=salt (mM)).

FIG. 5. The helicase (a) can control the movement of DNA (b) in at least two modes of operation. The helicase moves along the DNA in the 3′-5′ direction, but the orientation of the DNA in the nanopore (c) (dependent on which end of the DNA is captured) means that the enzyme can be used to either move the DNA out of the nanopore against the applied field (FIG. 5b), or move the DNA into the nanopore with the applied field (FIG. 5a). FIG. 5b) When the 5′ end of the DNA is captured the helicase works against the direction of the field applied by the voltage, pulling the threaded DNA out of the nanopore until the DNA is ejected back to the cis chamber. On the right is an example DNA-helicase event from Hel308 running 5′down against the applied field (y-axis=current (pA), x-axis=time (s)). FIG. 5a) When the DNA is captured 3′-down in the nanopore, the enzyme moves the DNA into the nanopore in the direction of the field until it is fully translocated through the pore and lost on the trans side of the bilayer. On the right is an example DNA-helicase event from Hel308 running 3′-down with the applied field (y-axis=current (pA), x-axis=time (s)). Current traces vary between the 5′down and 3′down orientations of DNA.

FIG. 6. Fluorescence assay for testing enzyme activity. a) A custom fluorescent substrate was used to assay the ability of the helicase (a) to displace hybridized dsDNA. 1) The fluorescent substrate strand (100 nM final) has a 3′ ssDNA overhang, and a 40 base section of hybridized dsDNA. The major upper strand (b) has a carboxyfluorescein base (c) at the 5′ end, and the hybridized complement (d) has a black-hole quencher (BHQ-1) base (e) at the 3′ end. When hybridized the fluorescence from the fluorescein is quenched by the local BHQ-1, and the substrate is essentially non-fluorescent. 1 μM of a capture strand (f) that is complementary to the shorter strand of the fluorescent substrate (d) is included in the assay. 2) In the presence of ATP (1 mM) and MgCl₂ (5 mM), helicase (100 nM) added to the substrate binds to the 3′ tail of the fluorescent substrate, moves along the major strand, and displaces the complementary strand as shown. 3) Once the complementary strand with BHQ-1 is fully displaced the fluorescein on the major strand fluoresces. 4) Excess of capture strand preferentially anneals to the complementary DNA to prevent re-annealing of initial substrate and loss of fluorescence. b) Graph of the initial rate of activity (y-axis, relative activity) in buffer solutions (10 mM Hepes pH 8.0, 1 mM ATP, 5 mM MgCl₂, 100 nM fluorescent substrate DNA, 1 μM capture DNA) containing different concentrations of KCl (x-axis, mM) from 400 mM to 2 M.

FIG. 7 shows examples of helicase controlled DNA events using different Hel308 helicases (y-axis=current (pA), x-axis=time (min) for FIGS. 7a-c, 180 mV, Hepes pH 8.0, 0.15 nM 400 mer DNA SEQ ID NOS: 59 and 60, 100 nM Hel308, 1 mM DTT, 1 mM ATP, 1 mM MgCl₂): Hel308 Mhu (a), Hel308 Mok (b) and Hel308 Mma (c). These represent typical examples of DNA controlled movement through MspA nanopores that ended at the polyT level.

FIG. 8. Fluorescence assay for testing helicase internal binding activity. A) Custom fluorescent substrates were used to assay the ability of the helicases to bind to DNA lacking native 3′ ends, allowing them to subsequently displace hybridized dsDNA. The fluorescent substrate strand (50 nM final) has a 3′ ssDNA overhang, and a 40 base section of hybridized dsDNA. The major upper strands (a) are modified with four consecutive non-DNA-derived triethylene glycol spacers (referred to as “spacer 9” groups, labelled b), either at the 3′ end, or internally, at the junction between the overhang and the dsDNA (as a negative control). Furthermore, the major upper strand has a carboxyfluorescein base (c) at the 5′ end, and the hybridized complement (d) has a black-hole quencher (BHQ-1) base (e) at the 3′ end. When hybridized, the fluorescence from the fluorescein is quenched by the local BHQ-1, and the substrate is essentially non-fluorescent. A capture strand (1 μM, f), that is complementary to the shorter strand (d) of the fluorescent substrate, is included in the assay. B) In the presence of ATP (1 mM) and MgCl₂ (1 mM), a Hel308 helicase homologue (20 nM, g), added to the substrate containing 3′-terminal “spacer 9” groups, can bind to the ssDNA overhang of the fluorescent substrate, move along the major strand, and displace the complementary strand. C) Once the complementary strand with BHQ-1 is fully displaced the fluorescein on the major strand fluoresces. D) An excess of capture strand preferentially anneals to the complementary DNA to prevent re-annealing of initial substrate and loss of fluorescence.

FIG. 9 shows the relative rates of Hel308-mediated dsDNA turnover comparing 3′-unmodified DNA and 3′-“spacer 9” DNA in 400 mM NaCl, 10 mM Hepes, pH 8.0, 1 mM ATP, 1 mM MgCl₂, 50 nM fluorescent substrate DNA, 1 μM capture DNA (y-axis=relative “3′-Sp9” activity (% wrt native 3′), x-axis=a (Mbu), b (Csy), c (Tga), d (Mma), e (Mhu), f (Min), g (Mig), h (Mmaz), i (Mac), j (Mok), k (Mth), l (Mba), m (Mzh)).

FIG. 10. Schematic of the use of a helicase to control DNA movement through a nanopore which is employed in example 5. A) A DNA substrate (SEQ ID NOs 67 (labelled w) and 68 (labelled x)) with an annealed primer (SEQ ID NO 69 (labelled y)) with an attached cholesterol-tag (labelled z) is added to the cis side of the bilayer. The cholesterol tag binds to the bilayer, enriching the substrate at the bilayer surface. Helicase (labelled l) added to the cis compartment binds to the 4 bp leader of SEQ ID NO 67. B) Under an applied voltage, the DNA substrate is captured by the nanopore via the 5′ leader section on the DNA, which strips off SEQ ID NO 69. C) Under the force of the applied field the DNA is pulled into the pore until the bound helicase (1) contacts the top of the pore and prevents further uncontrolled translocation. In this process the antisense strand SEQ ID NO 68 is stripped from the DNA strand. D) In the presence of divalent metal ions and NTP substrate, the helicase (l) on top of the pore moves along the DNA and controls the translocation of the DNA through the pore. The helicase movement along the DNA in a 3′ to 5′ direction pulls the threaded DNA out of the pore against the applied field. The exposed single stranded DNA on the cis side (3′ in this case) is available for further helicases (2-4) to bind either at the terminal nucleotide or at an internal nucleotide. E) If the helicase at the pore (1) disengages from the DNA, the DNA is pulled into the pore by the field until the next helicase (2) on the DNA reaches the pore. The helicase at the pore pulls the DNA out of the nanopore, feeding it back to the cis compartment. The last section of DNA to pass through the nanopore is the 5′-leader. F) When the helicase moves the DNA out of the nanopore it is lost back to the cis compartment. Arrows indicate the direction of DNA movement.

FIG. 11 shows data plots (y-axis=position in the 900 mer, x-axis=index) which indicate how the position of the region of DNA in the nanopore of the 900 mer (y-axis) varied as the Hel308 helicase homologue Mbu controlled the translocation of the DNA strand through the MspA pore (x-axis) during each helicase event. A-C show examples of typical translocation events of the entire DNA strand from approximately the beginning of the strand through to the end of the strand (exiting via polyT leader), whereas event D shows an example of incomplete DNA translocation, where enzyme detachment means the DNA never makes it to the end of the strand. The slips (eg. such as the large slips highlighted by dotted circles) indicate the sequence falling back to a previous point in the strand, and are the result of enzyme detachment. When an enzyme detaches the DNA will be pulled back under the force of the field into the nanopore until another enzyme further along the strand contacts the pore, then continuing helicase movement.

FIG. 12 shows data plots (y-axis=position in the 900 mer, x-axis=index) which indicate how the position of the 900 mer varied as the Hel308 helicase homologue Tga controlled the translocation of the DNA strand through the MspA pore. Events A-D show translocation of the entire DNA strand.

FIG. 13 shows a fluorescence assay used to compare the enzyme processivity of Hel308 Mbu helicase (SEQ ID NO: 10) to that of Hel 308 Mok helicase (SEQ ID NO: 29). A custom fluorescent substrate was used to assay the ability of the helicase to displace hybridized dsDNA. The fluorescent substrate (50 nM final) has a 3′ ssDNA overhang, and 80 (a1) and 33 base-pair (a2) sections of hybridized dsDNA (section A, SEQ ID NO: 70). The major bottom “template” strand (1) is hybridized to an 80 nt “blocker” strand (2, SEQ ID NO: 71), adjacent to its 3′ overhang, and a 33 nt fluorescent probe (3, SEQ ID NO: 72), labelled at its 5′ and 3′ ends with carboxyfluorescein (FAM) (4) and black-hole quencher (BHQ-1) (5) bases, respectively. When hybridized, the FAM is distant from the BHQ-1 and the substrate is essentially fluorescent. In the presence of ATP (1 mM) and MgCl₂ (10 mM) (addition of ATP and MgCl2 is indicated by 7 and 8 respectively), the helicase (6, 20 nM) binds to the substrate's 3′ overhang (SEQ ID NO: 70), moves along the lower strand, and begins to displace the 80 nt blocker strand (SEQ ID NO: 71), as shown in section B. If processive, the helicase displaces the fluorescent probe too (section C, SEQ ID NO: 72, labeled with a carboxyfluorescein (FAM) at its 5′ end a black-hole quencher (BHQ-1) at its 3′ end). The fluorescent probe is designed in such a way that its 5′ and 3′ ends are self-complementary and thus form a kinetically-stable hairpin once displaced, preventing the probe from re-annealing to the template strand (section D). Upon formation of the hairpin product, the FAM is brought into the vicinity of the BHQ-1 and its fluorescence is quenched. A processive enzyme, capable of displacing the 80 mer “blocker” (SEQ ID NO: 71) and fluorescent (SEQ ID NO: 72, labeled with a carboxyfluorescein (FAM) at its 5′ end a black-hole quencher (BHQ-1) at its 3′ end) strands will therefore lead to a decrease in fluorescence over time. However, if the enzyme has a processivity of less than 80 nt it would be unable to displace the fluorescent strand (SEQ ID NO. 72, labeled with a carboxyfluorescein (FAM) at its 5′ end a black-hole quencher (BHQ-1) at its 3′ end) and, therefore, the “blocker” strand (SEQ ID NO: 71) would reanneal to the major bottom strand (section E).

FIG. 14 shows additional custom fluorescent substrates which were also used for control purposes. The substrate used as a negative control was identical to that of the one described in FIG. 3 but lacking the 3′overhang (section A, (SEQ ID NOs: 71 (labelled 1), 72 (labelled 2) (labeled with a carboxyfluorescein (FAM) (3) at its 5′ end a black-hole quencher (BHQ-1) (4) at its 3′ end) and 73 (labelled 5, consisting of an 80 bp section (a1) and a 33 bp section (a2)))). A similar substrate to that described in FIG. 3 but lacking the 80 base pair section (SEQ ID NOs: 72 (labelled 2) (labeled with a carboxyfluorescein (FAM) (3) at its 5′ end a black-hole quencher (BHQ-1) (4) at its 3′ end) and 74 (labelled 6, consisting of a 28 bp section (a3))), was used as a positive control for active, but not necessarily processive, helicases (section B).

FIG. 15 shows a graph (y-axis=fluorescence (arbitrary units), x-axis=time (min)) of the time-dependent fluorescence changes upon testing Hel308 Mbu helicase (SEQ ID NO: 10, empty circles) and Hel 308 Mok helicase (SEQ ID NO: 29, black triangles) against the processivity substrate shown in FIG. 13 in buffered solution (400 mM NaCl, 10 mM Hepes pH 8.0, 1 mM ATP, 10 mM MgCl₂, 50 nM fluorescent substrate DNA (SEQ ID NOs: 70, 71 and 72 (labeled with a carboxyfluorescein (FAM) at its 5′ end a black-hole quencher (BHQ-1) at its 3′ end). The decrease in fluorescence exhibited by Hel308 Mok denotes the increased processivity of these complexes as compared to Hel308 Mbu (SEQ ID NO: 10).

FIG. 16 shows a graph (y-axis=fluorescence (arbitrary units), x-axis=time (min)) of the time-dependent fluorescence changes upon testing Hel308 Mbu helicase (SEQ ID NO: 10, empty circles) and Hel 308 Mok helicase (SEQ ID NO: 29, black triangles) against the positive control processivity substrate (shown in FIG. 14 section B, SEQ ID NOs: 72 (labeled with a carboxyfluorescein (FAM) at its 5′ end a black-hole quencher (BHQ-1) at its 3′ end) and 74) in buffered solution (400 mM NaCl, 10 mM Hepes pH 8.0, 1 mM ATP, 10 mM MgCl₂, 50 nM fluorescent substrate DNA (SEQ ID NOs: 72 (labeled with a carboxyfluorescein (FAM) at its 5′ end a black-hole quencher (BHQ-1) at its 3′ end) and 74)). This positive control demonstrated that both helicases were indeed active, as denoted by a fluorescence decrease for both samples.

### DESCRIPTION OF THE SEQUENCE LISTING

SEQ ID NO: 1 shows the codon optimized polynucleotide sequence encoding the MS-B1 mutant MspA monomer. This mutant lacks the signal sequence and includes the following mutations: D90N, D91N, D93N, D118R, D134R and E139K.

SEQ ID NO: 2 shows the amino acid sequence of the mature form of the MS-B1 mutant of the MspA monomer. This mutant lacks the signal sequence and includes the following mutations: D90N, D91N, D93N, D118R, D134R and E139K.

SEQ ID NO: 3 shows the polynucleotide sequence encoding one subunit of α-hemolysin-E111N/K147N (α-HL-NN; Stoddart et al., PNAS, 2009; 106(19): 7702-7707).

SEQ ID NO: 4 shows the amino acid sequence of one subunit of α-HL-NN.

SEQ ID NOs: 5 to 7 shows the amino acid sequences of MspB, C and D.

SEQ ID NO: 8 shows the amino acid sequence of the Hel308 motif.

SEQ ID NO: 9 shows the amino acid sequence of the extended Hel308 motif.

SEQ ID NOs: 10 to 58 show the amino acid sequences of the Hel308 helicases and motifs in Table 5.

SEQ ID NOs: 59 to 74 show the sequences used in the Examples.

SEQ ID NO: 75 shows the sequence of Hel308 Dth in the alignment on on page 57 onwards.

SEQ ID NO: 76 shows the sequence of Hel308 Mmar in the alignment on on page 57 onwards.

SEQ ID NO: 77 shows the sequence of Hel308 Nth in the alignment on page 57 onwards.

SEQ ID NO: 78 shows the consensus sequence in the alignment on page 57 onwards.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a pore” includes two or more such pores, reference to “a helicase” includes two or more such helicases, reference to “a polynucleotide” includes two or more such polynucleotides, and the like.

All publications, patents and patent applications cited herein, whether supra or infra, are hereby incorporated by reference in their entirety.

### Hel308 Methods of the Invention

The invention provides a method of characterising a target polynucleotide. The method comprises contacting the target polynucleotide with a transmembrane pore and a Hel308 helicase such that the helicase controls the movement of the target polynucleotide through the pore and nucleotides in the target polynucleotide interact with the pore. One or more characteristics of the target polynucleotide are then measured using standard methods known in the art. Steps (a) and (b) are preferably carried out with a potential applied across the pore. As discussed in more detail below, the applied potential typically results in the formation of a complex between the pore and the helicase. The applied potential may be a voltage potential. Alternatively, the applied potential may be a chemical potential. An example of this is using a salt gradient across the lipid membrane. A salt gradient is disclosed in Holden et al., J Am Chem Soc. 2007 Jul. 11; 129(27):8650-5.

In some instances, the current passing through the pore during one or more interactions is used to determine the sequence of the target polynucleotide. This is Strand Sequencing.

The method has several advantages. First, the inventors have surprisingly shown that Hel308 helicases have a surprisingly high salt tolerance and so the method of the invention may be carried out at high salt concentrations. In the context of Strand Sequencing, a charge carrier, such as a salt, is necessary to create a conductive solution for applying a voltage offset to capture and translocate the target polynucleotide and to measure the resulting sequence-dependent current changes as the polynucleotide passes through the pore. Since the measurement signal is dependent on the concentration of the salt, it is advantageous to use high salt concentrations to increase the magnitude of the acquired signal. High salt concentrations provide a high signal to noise ratio and allow for currents indicative of the presence of a nucleotide to be identified against the background of normal current fluctuations. For Strand Sequencing, salt concentrations in excess of 100 mM are ideal and salt concentrations of 1 M and above are preferred. The inventors have surprisingly shown that Hel308 helicases will function effectively at salt concentrations as high as, for example, 2 M.

Second, when a voltage is applied, Hel308 helicases can surprisingly move the target polynucleotide in two directions, namely with or against the field resulting from the applied voltage. Hence, the method of the invention may be carried out in one of two preferred modes. Different signals are obtained depending on the direction the target polynucleotide moves through the pore, ie in the direction of or against the field. This is discussed in more detail below.

Third, Hel308 helicases typically move the target polynucleotide through the pore one nucleotide at a time. Hel308 helicases can therefore function like a single-base ratchet. This is of course advantageous when sequencing a target polynucleotide because substantially all, if not all, of the nucleotides in the target polynucleotide may be identified using the pore.

Fourth, Hel308 helicases are capable of controlling the movement of single stranded polynucleotides and double stranded polynucleotides. This means that a variety of different target polynucleotides can be characterised in accordance with the invention.

Fifth, Hel308 helicases appear very resistant to the field resulting from applied voltages. The inventors have seen very little movement of the polynucleotide under an “unzipping” condition. This is important because it means that there are no complications from unwanted “backwards” movements when moving polynucleotides against the field resulting from an applied voltage.

Sixth, Hel308 helicases are easy to produce and easy to handle. Their use therefore contributed to a straightforward and less expensive method of sequencing.

The method of the invention is for characterising a target polynucleotide. A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the target polynucleotide can be oxidized or methylated. One or more nucleotides in the target polynucleotide may be damaged. One or more nucleotides in the target polynucleotide may be modified, for instance with a label or a tag. The target polynucleotide may comprise one or more spacers.

A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), guanosine monophosphate (GMP), thymidine monophosphate (TMP), uridine monophosphate (UMP), cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP) and deoxycytidine monophosphate (dCMP). The nucleotides are preferably selected from AMP, TMP, GMP, CMP, UMP, dAMP, dTMP, dGMP or dCMP.

A nucleotide may be abasic (i.e. lack a nucleobase).

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains.

The whole or only part of the target polynucleotide may be characterised using this method. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The organism or microorganism is typically archaean, prokaryotic or eukaryotic and typically belongs to one the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs. Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton.

The sample may be a non-biological sample. The non-biological sample is preferably a fluid sample. Examples of a non-biological sample include surgical fluids, water such as drinking water, sea water or river water, and reagents for laboratory tests.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

A transmembrane pore is a structure that permits hydrated ions driven by an applied potential to flow from one side of the membrane to the other side of the membrane.

Any membrane may be used in accordance with the invention. Suitable membranes are well-known in the art. The membrane is preferably an amphiphilic layer. An amphiphilic layer is a layer formed from amphiphilic molecules, such as phospholipids, which have both hydrophilic and lipophilic properties. The amphiphilic layer may be a monolayer or a bilayer.

The membrane is preferably a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Example. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

In a preferred embodiment, the lipid bilayer is formed as described in International Application No. PCT/GB08/004127 (published as WO 2009/077734).

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂0₃, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in International Application No. PCT/US2008/010637 (published as WO 2009/035647).

The method is typically carried out using (i) an artificial bilayer comprising a pore, (ii) an isolated, naturally-occurring lipid bilayer comprising a pore, or (iii) a cell having a pore inserted therein. The method is preferably carried out using an artificial lipid bilayer. The bilayer may comprise other transmembrane and/or intramembrane proteins as well as other molecules in addition to the pore. Suitable apparatus and conditions are discussed below. The method of the invention is typically carried out in vitro.

The polynucleotide may be coupled to the membrane. This may be done using any known method. If the membrane is an amphiphilic layer, such as a lipid bilayer (as discussed in detail above), the polynucleotide is preferably coupled to the membrane via a polypeptide present in the membrane or a hydrophobic anchor present in the membrane. The hydrophobic anchor is preferably a lipid, fatty acid, sterol, carbon nanotube or amino acid.

The polynucleotide may be coupled directly to the membrane. The polynucleotide is preferably coupled to the membrane via a linker. Preferred linkers include, but are not limited to, polymers, such as polynucleotides, polyethylene glycols (PEGs) and polypeptides. If a polynucleotide is coupled directly to the membrane, then some data will be lost as the characterising run cannot continue to the end of the polynucleotide due to the distance between the membrane and the helicase. If a linker is used, then the polynucleotide can be processed to completion. If a linker is used, the linker may be attached to the polynucleotide at any position. The linker is preferably attached to the polynucleotide at the tail polymer.

The coupling may be stable or transient. For certain applications, the transient nature of the coupling is preferred. If a stable coupling molecule were attached directly to either the 5′ or 3′ end of a polynucleotide, then some data will be lost as the characterising run cannot continue to the end of the polynucleotide due to the distance between the bilayer and the helicase's active site. If the coupling is transient, then when the coupled end randomly becomes free of the bilayer, then the polynucleotide can be processed to completion. Chemical groups that form stable or transient links with the membrane are discussed in more detail below. The polynucleotide may be transiently coupled to an amphiphilic layer or lipid bilayer using cholesterol or a fatty acyl chain. Any fatty acyl chain having a length of from 6 to 30 carbon atoms, such as hexadecanoic acid, may be used.

In preferred embodiments, polynucleotide is coupled to a lipid bilayer. Coupling of polynucleotides to synthetic lipid bilayers has been carried out previously with various different tethering strategies. These are summarized in Table 1 below.

TABLE 1

| Attachment | Type of | |
|---|---|---|
| group | coupling | Reference |
| Thiol | Stable | Yoshina-Ishii, C. and S. G. Boxer (2003). |
| | | “Arrays of mobile tethered vesicles |
| | | on supported lipid bilayers.” |
| | | J Am Chem Soc 125(13): 3696-7. |
| Biotin | Stable | Nikolov, V., R. Lipowsky, et al. (2007). |
| | | “Behavior of giant vesicles with |
| | | anchored DNA molecules.” |
| | | Biophys J 92(12): 4356-68 |
| Cholestrol | Transient | Pfeiffer, I. and F. Hook (2004). “Bivalent |
| | | cholesterol-based coupling of oligonucletides |
| | | to lipid membrane assemblies.” |
| | | J Am Chem Soc 126(33): 10224-5 |
| Lipid | Stable | van Lengerich, B., R. J. Rawle, et al. |
| | | “Covalent attachment of lipid vesicles |
| | | to a fluid-supported bilayer allows |
| | | observation of DNA-mediated vesicle |
| | | interactions.” Langmuir 26(11): |
| | | 8666-72 |

Polynucleotides may be functionalized using a modified phosphoramidite in the synthesis reaction, which is easily compatible for the addition of reactive groups, such as thiol, cholesterol, lipid and biotin groups. These different attachment chemistries give a suite of attachment options for polynucleotides. Each different modification group tethers the polynucleotide in a slightly different way and coupling is not always permanent so giving different dwell times for the polynucleotide to the bilayer. The advantages of transient coupling are discussed above.

Coupling of polynucleotides can also be achieved by a number of other means provided that a reactive group can be added to the polynucleotide. The addition of reactive groups to either end of DNA has been reported previously. A thiol group can be added to the 5′ of ssDNA using polynucleotide kinase and ATPγS (Grant, G. P. and P. Z. Qin (2007). “A facile method for attaching nitroxide spin labels at the 5′ terminus of nucleic acids.” Nucleic Acids Res 35(10): e77). A more diverse selection of chemical groups, such as biotin, thiols and fluorophores, can be added using terminal transferase to incorporate modified oligonucleotides to the 3′ of ssDNA (Kumar, A., P. Tchen, et al. (1988). “Nonradioactive labeling of synthetic oligonucleotide probes with terminal deoxynucleotidyl transferase.” Anal Biochem 169(2): 376-82).

Alternatively, the reactive group could be considered to be the addition of a short piece of DNA complementary to one already coupled to the bilayer, so that attachment can be achieved via hybridization. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Acad Sci USA 89(20): 9823-5). Alternatively either ssDNA or dsDNA could be ligated to native dsDNA and then the two strands separated by thermal or chemical denaturation. To native dsDNA, it is possible to add either a piece of ssDNA to one or both of the ends of the duplex, or dsDNA to one or both ends. Then, when the duplex is melted, each single strand will have either a 5′ or 3′ modification if ssDNA was used for ligation or a modification at the 5′ end, the 3′ end or both if dsDNA was used for ligation. If the polynucleotide is a synthetic strand, the coupling chemistry can be incorporated during the chemical synthesis of the polynucleotide. For instance, the polynucleotide can be synthesized using a primer a reactive group attached to it.

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. By using an antisense primer that has a reactive group, such as a cholesterol, thiol, biotin or lipid, each copy of the target DNA amplified will contain a reactive group for coupling.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as 6, 7 or 8 subunits. The pore is more preferably a heptameric or octameric pore. The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin. anthrax toxin and leukocidins, and outer membrane proteins/porins of bacteria, such as Mycobacterium smegmatis porin (Msp), for example MspA, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from Msp or from α-hemolysin (α-HL).

The transmembrane protein pore is preferably derived from Msp, preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. Preferably the pore is derived from MspA or a homolog or paralog thereof.

A monomer derived from Msp comprises the sequence shown in SEQ ID NO: 2 or a variant thereof. SEQ ID NO: 2 is the MS-(B1)8 mutant of the MspA monomer. It includes the following mutations: D90N, D91N, D93N, D118R, D134R and E139K. A variant of SEQ ID NO: 2 is a polypeptide that has an amino acid sequence which varies from that of SEQ ID NO: 2 and which retains its ability to form a pore. The ability of a variant to form a pore can be assayed using any method known in the art. For instance, the variant may be inserted into a lipid bilayer along with other appropriate subunits and its ability to oligomerise to form a pore may be determined. Methods are known in the art for inserting subunits into membranes, such as lipid bilayers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

Over the entire length of the amino acid sequence of SEQ ID NO: 2, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

SEQ ID NO: 2 is the MS-(B1)8 mutant of the MspA monomer. The variant may comprise any of the mutations in the MspB, C or D monomers compared with MspA. The mature forms of MspB, C and D are shown in SEQ ID NOs: 5 to 7. In particular, the variant may comprise the following substitution present in MspB: A138P. The variant may comprise one or more of the following substitutions present in MspC: A96G, N102E and A138P. The variant may comprise one or more of the following mutations present in MspD: Deletion of G1, L2V, ESQ, L8V, D13G, W21A, D22E, K47T, I49H, I68V, D91G, A96Q, N102D, S103T, V104I, S136K and G141A. The variant may comprise combinations of one or more of the mutations and substitutions from Msp B, C and D. The variant preferably comprises the mutation L88N. The variant of SEQ ID NO: 2 has the mutation L88N in addition to all the mutations of MS-B1 and is called MS-B2. The pore used in the invention is preferably MS-(B2)8.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 2 below. Where amino acids have similar polarity, this can also be determined by reference to the hydropathy scale for amino acid side chains in Table 3.

TABLE 2
Chemical properties of amino acids

| | |
|---|---|
| Ala | aliphatic, hydrophobic, neutral |
| Cys | polar, hydrophobic, neutral |
| Asp | polar, hydrophilic, charged (−) |
| Glu | polar, hydrophilic, charged (−) |
| Phe | aromatic, hydrophobic, neutral |
| Gly | aliphatic, neutral |
| His | aromatic, polar, hydrophilic, |
| | charged (+) |
| Ile | aliphatic, hydrophobic, neutral |
| Lys | polar, hydrophilic, charged(+) |
| Leu | aliphatic, hydrophobic, neutral |
| Met | hydrophobic, neutral |
| Asn | polar, hydrophilic, neutral |
| Pro | hydrophobic, neutral |
| Gln | polar, hydrophilic, neutral |
| Arg | polar, hydrophilic, charged (+) |
| Ser | polar, hydrophilic, neutral |
| Thr | polar, hydrophilic, neutral |
| Val | aliphatic, hydrophobic, neutral |
| Trp | aromatic, hydrophobic, neutral |
| Tyr | aromatic, polar, hydrophobic |

TABLE 3
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may include fragments of SEQ ID NO: 2. Such fragments retain pore forming activity. Fragments may be at least 50, 100, 150 or 200 amino acids in length. Such fragments may be used to produce the pores. A fragment preferably comprises the pore forming domain of SEQ ID NO: 2. Fragments must include one of residues 88, 90, 91, 105, 118 and 134 of SEQ ID NO: 2. Typically, fragments include all of residues 88, 90, 91, 105, 118 and 134 of SEQ ID NO: 2.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2 and which retains its ability to form a pore. A variant typically contains the regions of SEQ ID NO: 2 that are responsible for pore formation. The pore forming ability of Msp, which contains a β-barrel, is provided by β-sheets in each subunit. A variant of SEQ ID NO: 2 typically comprises the regions in SEQ ID NO: 2 that form β-sheets. One or more modifications can be made to the regions of SEQ ID NO: 2 that form β-sheets as long as the resulting variant retains its ability to form a pore. A variant of SEQ ID NO: 2 preferably includes one or more modifications, such as substitutions, additions or deletions. within its α-helices and/or loop regions.

The monomers derived from Msp may be modified to assist their identification or purification, for example by the addition of histidine residues (a hist tag), aspartic acid residues (an asp tag), a streptavidin tag or a flag tag, or by the addition of a signal sequence to promote their secretion from a cell where the polypeptide does not naturally contain such a sequence. An alternative to introducing a genetic tag is to chemically react a tag onto a native or engineered position on the pore. An example of this would be to react a gel-shift reagent to a cysteine engineered on the outside of the pore. This has been demonstrated as a method for separating hemolysin hetero-oligomers (Chem Biol. 1997 July; 4(7):497-505).

The monomer derived from Msp may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

The monomer derived from Msp may also be produced using D-amino acids. For instance, the monomer derived from Msp may comprise a mixture of L-amino acids and D-amino acids. This is conventional in the art for producing such proteins or peptides.

The monomer derived from Msp contains one or more specific modifications to facilitate nucleotide discrimination. The monomer derived from Msp may also contain other non-specific modifications as long as they do not interfere with pore formation. A number of non-specific side chain modifications are known in the art and may be made to the side chains of the monomer derived from Msp. Such modifications include, for example, reductive alkylation of amino acids by reaction with an aldehyde followed by reduction with NaBH₄, amidination with methylacetimidate or acylation with acetic anhydride.

The monomer derived from Msp can be produced using standard methods known in the art. The monomer derived from Msp may be made synthetically or by recombinant means. For example, the pore may be synthesized by in vitro translation and transcription (IVTT). Suitable methods for producing pores are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are discussed.

The transmembrane protein pore is also preferably derived from α-hemolysin (α-HL). The wild type α-HL pore is formed of seven identical monomers or subunits (i.e. it is heptameric). The sequence of one monomer or subunit of α-hemolysin-NN is shown in SEQ ID NO: 4. The transmembrane protein pore preferably comprises seven monomers each comprising the sequence shown in SEQ ID NO: 4 or a variant thereof. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 294 of SEQ ID NO: 4 form loop regions. Residues 113 and 147 of SEQ ID NO: 4 form part of a constriction of the barrel or channel of α-HL.

In such embodiments, a pore comprising seven proteins or monomers each comprising the sequence shown in SEQ ID NO: 4 or a variant thereof are preferably used in the method of the invention. The seven proteins may be the same (homoheptamer) or different (heteroheptamer).

A variant of SEQ ID NO: 4 is a protein that has an amino acid sequence which varies from that of SEQ ID NO: 4 and which retains its pore forming ability. The ability of a variant to form a pore can be assayed using any method known in the art. For instance, the variant may be inserted into a lipid bilayer along with other appropriate subunits and its ability to oligomerise to form a pore may be determined. Methods are known in the art for inserting subunits into membranes, such as lipid bilayers. Suitable methods are discussed above.

The variant may include modifications that facilitate covalent attachment to or interaction with the helicase. The variant preferably comprises one or more reactive cysteine residues that facilitate attachment to the helicase. For instance, the variant may include a cysteine at one or more of positions 8, 9, 17, 18, 19, 44, 45, 50, 51, 237, 239 and 287 and/or on the amino or carboxy terminus of SEQ ID NO: 4. Preferred variants comprise a substitution of the residue at position 8, 9, 17, 237, 239 and 287 of SEQ ID NO: 4 with cysteine (A8C, T9C, N17C, K237C, S239C or E287C). The variant is preferably any one of the variants described in International Application No. PCT/GB09/001690 (published as WO 2010/004273), PCT/GB09/001679 (published as WO 2010/004265) or PCT/GB10/000133 (published as WO 2010/086603).

The variant may also include modifications that facilitate any interaction with nucleotides.

The variant may be a naturally occurring variant which is expressed naturally by an organism, for instance by a Staphylococcus bacterium. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 4, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 4 over the entire sequence. There may be at least 80%, for example at least 85%, 90% or 95%, amino acid identity over a stretch of 200 or more, for example 230, 250, 270 or 280 or more, contiguous amino acids (“hard homology”). Homology can be determined as discussed above.

Amino acid substitutions may be made to the amino acid sequence of SEQ ID NO: 4 in addition to those discussed above, for example up to 1, 2, 3, 4, 5, 10, 20 or 30 substitutions. Conservative substitutions may be made as discussed above.

One or more amino acid residues of the amino acid sequence of SEQ ID NO: 4 may additionally be deleted from the polypeptides described above. Up to 1, 2, 3, 4, 5, 10, 20 or 30 residues may be deleted, or more.

Variants may be fragments of SEQ ID NO: 4. Such fragments retain pore-forming activity. Fragments may be at least 50, 100, 200 or 250 amino acids in length. A fragment preferably comprises the pore-forming domain of SEQ ID NO: 4. Fragments typically include residues 119, 121, 135. 113 and 139 of SEQ ID NO: 4.

One or more amino acids may be alternatively or additionally added to the polypeptides described above. An extension may be provided at the amino terminus or carboxy terminus of the amino acid sequence of SEQ ID NO. 4 or a variant or fragment thereof. The extension may be quite short, for example from I to 10 amino acids in length. Alternatively, the extension may be longer, for example up to 50 or 100 amino acids. A carrier protein may be fused to a pore or variant.

As discussed above, a variant of SEQ ID NO: 4 is a subunit that has an amino acid sequence which varies from that of SEQ ID NO: 4 and which retains its ability to form a pore. A variant typically contains the regions of SEQ ID NO: 4 that are responsible for pore formation. The pore forming ability of α-HL, which contains a β-barrel, is provided by β-strands in each subunit. A variant of SEQ ID NO: 4 typically comprises the regions in SEQ ID NO: 4 that form β-strands. The amino acids of SEQ ID NO: 4 that form β-strands are discussed above. One or more modifications can be made to the regions of SEQ ID NO: 4 that form β-strands as long as the resulting variant retains its ability to form a pore. Specific modifications that can be made to the β-strand regions of SEQ ID NO: 4 are discussed above.

A variant of SEQ ID NO: 4 preferably includes one or more modifications, such as substitutions, additions or deletions, within its α-helices and/or loop regions. Amino acids that form α-helices and loops are discussed above.

The variant may be modified to assist its identification or purification as discussed above.

Pores derived from α-HL can be made as discussed above with reference to pores derived from Msp.

In some embodiments, the transmembrane protein pore is chemically modified. The pore can be chemically modified in any way and at any site. The transmembrane protein pore is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The transmembrane protein pore may be chemically modified by the attachment of any molecule. For instance, the pore may be chemically modified by attachment of a dye or a fluorophore.

Any number of the monomers in the pore may be chemically modified. One or more, such as 2, 3, 4, 5, 6, 7, 8, 9 or 10, of the monomers is preferably chemically modified as discussed above.

The reactivity of cysteine residues may be enhanced by modification of the adjacent residues. For instance, the basic groups of flanking arginine, histidine or lysine residues will change the pKa of the cysteines thiol group to that of the more reactive S⁻ group. The reactivity of cysteine residues may be protected by thiol protective groups such as dTNB. These may be reacted with one or more cysteine residues of the pore before a linker is attached.

The molecule (with which the pore is chemically modified) may be attached directly to the pore or attached via a linker as disclosed in International Application Nos. PCT/GB09/001690 (published as WO 2010/004273), PCT/GB09/001679 (published as WO 2010/004265) or PCT/GB10/000133 (published as WO 2010/086603).

Any Hel308 helicase may be used in accordance with the invention. Hel308 helicases are also known as ski2-like helicases and the two terms can be used interchangeably.

The Hel308 helicase typically comprises the amino acid motif Q-X1-X2-G-R-A-G-R (hereinafter called the Hel308 motif, SEQ ID NO: 8). The Hel308 motif is typically part of the helicase motif VI (Tuteja and Tuteja, Eur. J. Biochem. 271, 1849-1863 (2004)). X1 may be C, M or L. X1 is preferably C. X2 may be any amino acid residue. X2 is typically a hydrophobic or neutral residue. X2 may be A, F, M, C, V, L, I, S, T, P or R. X2 is preferably A, F, M, C, V, L, I, S, T or P. X2 is more preferably A, M or L. X2 is most preferably A or M.

The Hel308 helicase preferably comprises the motif Q-X1-X2-G-R-A-G-R-P (hereinafter called the extended Hel308 motif; SEQ ID NO: 9) wherein X1 and X2 are as described above.

The most preferred Hel308 motifs and extended Hel308 motifs are shown in Table 5 below. The Hel308 helicase may comprise any of these preferred motifs.

The Hel308 helicase is preferably one of the helicases shown in Table 4 below or a variant thereof.

TABLE 4
Preferred Hel308 helicases

| Accession | Description |
|---|---|
| NP_578406.1 | ski2-like helicase [Pyrococcus furiosus DSM 3638] >sp\|O73946.1\|HELS_PYRFU |
| | RecName: Full = Putative ski2-type helicase >pdb\|2ZJ2\|A Chain A, Archaeal Dna |
| | Helicase Hjm Apo State In Form 1 >pdb\|2ZJ5\|A Chain A, Archaeal Dna Helicase |
| | Hjm Complexed With Adp In Form 1 >pdb\|2ZJ8\|A Chain A, Archaeal Dna |
| | Helicase Hjm Apo State In Form 2 >pdb\|2ZJA\|A Chain A, Archaeal Dna Helicase |
| | Hjm Complexed With Amppcp In Form 2 >dbj\|BAA32016.1\| helicase |
| | [Pyrococcus furiosus] >gb\|AAL80801.1\| helicase [Pyrococcus furiosus DSM |
| | 3638] |
| NP_126564.1 | ski2-like helicase [Pyrococcus abyssi GE5] >sp\|Q9V0A9.1\|HELS_PYRAB |
| | RecName: Full = Putative ski2-type helicase >emb\|CAB49795.1\| DNA helicase |
| | [Pyrococcus abyssi GE5] |
| NP_143168.1 | ski2-like helicase [Pyrococcus horikoshii OT3] >sp\|O59025.1\|HELS_PYRHO |
| | RecName: Full = Putative ski2-type helicase >dbj\|BAA30383.1\| 715aa long |
| | hypothetical protein [Pyrococcus horikoshii OT3] |
| YP_004424773.1 | ski2-like helicase [Pyrococcus sp. NA2] >gb\|AEC52769.1\| ski2-like helicase |
| | [Pyrococcus sp. NA2] |
| YP_004623750.1 | ski2-like helicase [Pyrococcus yayanosii CH1] >gb\|AEH24478.1\| ski2-like |
| | helicase [Pyrococcus yayanosii CH1] |
| YP_002307730.1 | ski2-like helicase [Thermococcus onnurineus NA1] >gb\|ACJ16833.1\| DNA |
| | helicase [Thermococcus onnurineus NA1] |
| YP_004763427.1 | ski2-like helicase [Thermococcus sp. 4557] >gb\|AEK73750.1\| ski2-like helicase |
| | [Thermococcus sp. 4557] |
| YP_002959236.1 | ski2-like helicase [Thermococcus gammatolerans EJ3] >gb\|ACS33372.1\| ski2- |
| | type helicase, putative [Thermococcus gammatolerans EJ3] |
| YP_004071709.1 | ski2-type helicase [Thermococcus barophilus MP] >gb\|ADT84486.1\| putative |
| | ski2-type helicase [Thermococcus barophilus MP] |
| YP_002994328.1 | Putative ski2-type helicase [Thermococcus sibiricus MM 739] >gb\|ACS89979.1\| |
| | Putative ski2-type helicase [Thermococcus sibiricus MM 739] |
| ZP_04875329.1 | Type III restriction enzyme, res subunit family [Aciduliprofundum boonei |
| | T469] >gb\|EDY35111.1\| Type III restriction enzyme, res subunit family |
| | [Aciduliprofundum boonei T469] |
| YP_003436565.1 | DEAD/DEAH box helicase [Ferroglobus placidus DSM 10642] >gb\|ADC66290.1\| |
| | DEAD/DEAH box helicase domain protein [Ferroglobus |
| | placidus DSM 10642] |
| YP_004485304.1 | ski2-type helicase [Methanotorris igneus Kol 5] >gb\|AEF97239.1\| ski2-type |
| | helicase [Methanotorris igneus Kol 5] |
| YP_004616424.1 | DEAD/DEAH box helicase domain-containing protein [Methanosalsum zhilinae |
| | DSM 4017] >gb\|AEH61205.1\| DEAD/DEAH box helicase domain protein |
| | [Methanosalsum zhilinae DSM 4017] |
| ZP_04873370.1 | Type III restriction enzyme, res subunit family [Aciduliprofundum boonei |
| | T469] >ref\|YP_003482774.1\| DEAD/DEAH box helicase domain protein |
| | [Aciduliprofundum boonei T469] >gb\|EDY36687.1\| Type III restriction enzyme, |
| | res subunit family [Aciduliprofundum boonei T469] >gb\|ADD08212.1\| |
| | DEAD/DEAH box helicase domain protein [Aciduliprofundum boonei T469] |
| YP_004342552.1 | ski2-type helicase [Archaeoglobus veneficus SNP6] >gb\|AEA47837.1\| ski2-type |
| | helicase [Archaeoglobus veneficus SNP6] |
| NP_071282.1 | SKI2-family helicase [Archaeoglobus fulgidus DSM 4304] |
| 2P6R_A | Chain A, Crystal Structure Of Superfamily 2 Helicase Hel308 In Complex With |
| | Unwound Dna >pdb\|2P6U\|A Chain A, Apo Structure Of The Hel308 Superfamily |
| | 2 Helicase |
| YP_685308.1 | ski2-like helicase [uncultured methanogenic archaeon RC-I] >sp\|Q0W6L1.1\|HELS_UNCMA |
| | RecName: Full = Putative ski2-type helicase >emb\|CAJ35982.1\| |
| | putative ski2-type helicase [uncultured methanogenic |
| | archaeon RC-I] |
| YP_001048404.1 | ski2-like helicase [Methanoculleus marisnigri JR1] >gb\|ABN58422.1\| |
| | DEAD/DEAH box helicase domain protein [Methanoculleus marisnigri JR1] |
| YP_919908.1 | DEAD/DEAH box helicase domain-containing protein [Thermofilum pendens |
| | Hrk 5] >gb\|ABL77905.1\| DEAD/DEAH box helicase domain protein |
| | [Thermofilum pendens Hrk 5] |
| YP_843229.1 | ski2-like helicase [Methanosaeta thermophila PT] >gb\|ABK14589.1\| |
| | DEAD/DEAH box helicase domain protein [Methanosaeta thermophila PT] |
| ZP_08045937.1 | ski2-like helicase [Haladaptatus paucihalophilus DX253] >gb\|EFW90585.1\| ski2- |
| | like helicase [Haladaptatus paucihalophilus DX253] |
| NP_280985.1 | ski2-like helicase [Halobacterium sp. NRC-1] >ref\|YP_001690117.1\| ski2-like |
| | helicase [Halobacterium salinarum R1] >sp\|Q9HMV6.1\|HELS_HALSA |
| | RecName: Full = Putative ski2-type helicase >sp\|B0R7Q2.1\|HELS_HALS3 |
| | RecName: Full = Putative ski2-type helicase >gb\|AAG20465.1\| DNA repair |
| | protein [Halobacterium sp. NRC-1] >emb\|CAP14771.1\| putative DNA helicase |
| | [Halobacterium salinarum R1] |
| YP_003357840.1 | Holliday junction migration helicase [Methanocella paludicola |
| | SANAE] >dbj\|BAI62857.1\| Holliday junction migration helicase |
| | [Methanocella paludicola SANAE] |
| YP_003457479.1 | DEAD/DEAH box helicase domain protein [Methanocaldococcus sp. |
| | FS406-22] >gb\|ADC68743.1\| DEAD/DEAH box helicase domain protein |
| | [Methanocaldococcus sp. FS406-22] |
| YP_003127632.1 | DEAD/DEAH box helicase domain protein [Methanocaldococcus fervens |
| | AG86] >gb\|ACV24132.1\| DEAD/DEAH box helicase domain protein |
| | [Methanocaldococcus fervens AG86] |
| YP_003735335.1 | ski2-like helicase [Halalkalicoccus jeotgali B3] >gb\|ADJ13543.1\| ski2-like |
| | helicase [Halalkalicoccus jeotgali B3] |
| YP_503885.1 | ski2-like helicase [Methanospirillum hungatei JF-1] >gb\|ABD42166.1\| |
| | DEAD/DEAH box helicase-like protein [Methanospirillum hungatei JF-1] |
| BAJ48115.1 | helicase [Candidates Caldiarchaeum subterraneum] >dbj\|BAJ48144.1\| helicase |
| | [Candidates Caldiarchaeum subterraneum] >dbj\|BAJ50919.1\| helicase |
| | [Candidates Caldiarchaeum subterraneum] |
| YP_001405615.1 | ski2-like helicase [Candidates Methanoregula boonei |
| | 6A8] >sp\|A7IB61.1\|HELS_METB6 RecName: Full = Putative ski2-type |
| | helicase >gb\|ABS56972.1\| DEAD/DEAH box helicase domain protein |
| | [Methanoregula boonei 6A8] |
| YP_306959.1 | ski2-like helicase [Methanosarcina barkeri str. Fusaro] >sp\|Q465R3.1\|HELS_METBF |
| | RecName: Full = Putative ski2-type helicase >gb\|AAZ72379.1\| |
| | helicase [Methanosarcina barkeri str. Fusaro] |
| YP_001031179.1 | ski2-like helicase [Methanocorpusculum labreanum Z] >gb\|ABN07912.1\| |
| | DEAD/DEAH box helicase domain protein [Methanocorpusculum labreanum Z] |
| YP_003541733.1 | DEAD/DEAH box helicase [Methanohalophilus mahii DSM 5219] >gb\|ADE36088.1\| |
| | DEAD/DEAH box helicase domain protein [Methanohalophilus mahii DSM 5219] |
| YP_004384692.1 | putative Ski2-type helicase [Methanosaeta concilii GP6] >gb\|AEB68874.1\| |
| | putative Ski2-type helicase [Methanosaeta concilii GP6] |
| YP_003725904.1 | DEAD/DEAH box helicase domain-containing protein [Methanohalobium |
| | evestigatum Z-7303] >gb\|ADI73108.1\| DEAD/DEAH box helicase domain |
| | protein [Methanohalobium evestigatum Z-7303] |
| YP_003405271.1 | DEAD/DEAH box helicase [Haloterrigena turkmenica DSM |
| | 5511] >gb\|ADB62598.1\| DEAD/DEAH box helicase domain protein |
| | [Haloterrigena turkmenica DSM 5511] |
| YP_004244914.1 | DEAD/DEAH box helicase [Vulcanisaeta moutnovskia 768-28] >gb\|ADY01412.1\| |
| | DEAD/DEAH box helicase domain protein [Vulcanisaeta |
| | moutnovskia 768-28] |
| YP_001540156.1 | DEAD/DEAH box helicase domain-containing protein [Caldivirga |
| | maquilingensis IC-167] >sp\|A8MB76.1\|HELS_CALMQ RecName: Full = Putative |
| | ski2-type helicase >gb\|ABW01166.1\| DEAD/DEAH box helicase domain protein |
| | [Caldivirga maquilingensis IC-167] |
| NP_618094.1 | ski2-like helicase [Methanosarcina acetivorans C2A] >sp\|Q8TL39.1\|HELS_METAC |
| | RecName: Full = Putative ski2-type helicase >gb\|AAM06574.1\| |
| | helicase [Methanosarcina acetivorans C2A] |
| YP_003900980.1 | DEAD/DEAH box helicase domain-containing protein [Vulcanisaeta distributa |
| | DSM 14429] >gb\|ADN49929.1\| DEAD/DEAH box helicase domain protein |
| | [Vulcanisaeta distributa DSM 14429] |
| YP_003896003.1 | DEAD/DEAH box helicase domain-containing protein [Methanoplanus |
| | petrolearius DSM 11571] >gb\|ADN37565.1\| DEAD/DEAH box helicase domain |
| | protein [Methanoplanus petrolearius DSM 11571] |
| YP_003615773.1 | DEAD/DEAH box helicase domain protein [Methanocaldococcus infernus |
| | ME] >gb\|ADG12809.1\| DEAD/DEAH box helicase domain protein |
| | [Methanocaldococcus infernus ME] |
| YP_183745.1 | RNA helicase Ski2-like protein [Thermococcus kodakarensis |
| | KOD1] >sp\|Q5JGV6.1\|HELS_PYRKO RecName: Full = Putative ski2-type |
| | helicase; Contains: RecName: Full = Endonuclease PI-PkoHel; AltName: |
| | Full = Pko Hel intein >dbj\|BAD85521.1\| RNA helicase Ski2 homolog |
| | [Thermococcus kodakarensis KOD1] |
| YP_001322557.1 | DEAD/DEAH box helicase domain-containing protein [Methanococcus vannielii |
| | SB] >sp\|A6UN73.1\|HELS_METVS RecName: Full = Putative ski2-type |
| | helicase >gb\|ABR53945.1\| DEAD/DEAH box helicase domain protein |
| | [Methanococcus vannielii SB] |
| YP_002467772.1 | ski2-like helicase [Methanosphaerula palustris E1-9c] >gb\|ACL18049.1\| |
| | DEAD/DEAH box helicase domain protein [Methanosphaerula palustris E1-9c] |
| YP_003480097.1 | DEAD/DEAH box helicase [Natrialba magadii ATCC 43099] >gb\|ADD05535.1\| |
| | DEAD/DEAH box helicase domain protein [Natrialba magadii ATCC 43099] |
| YP_004577043.1 | ski2-type helicase [Methanothermococcus okinawensis IH1] >gb\|AEH07265.1\| |
| | ski2-type helicase [Methanothermococcus okinawensis IH1] |
| YP_004742641.1 | superfamily II helicase [Methanococcus maripaludis XI] >gb\|AEK19898.1\| |
| | superfamily II helicase [Methanococcus maripaludis X1] |
| NP_632449.1 | ski2-like helicase [Methanosarcina mazei Go1] >sp\|Q8PZR7.1\|HELS_METMA |
| | RecName: Full = Putative ski2-type helicase >gb\|AAM30121.1\| helicase |
| | [Methanosarcina mazei Go1] |
| YP_001097223.1 | DEAD/DEAH box helicase domain-containing protein [Methanococcus |
| | maripaludis C5] >gb\|ABO35008.1\| DEAD/DEAH box helicase domain protein |
| | [Methanococcus maripaludis C5] |
| YP_004742247.1 | DEAD/DEAH box helicase domain-containing protein [Methanococcus |
| | maripaludis XI] >gb\|AEK19504.1\| DEAD/DEAH box helicase domain-containing |
| | protein [Methanococcus maripaludis X1] |
| YP_004794766.1 | ski2-like helicase [Haloarcula hispanica ATCC 33960] >gb\|AEM55778.1\| ski2- |
| | like helicase [Haloarcula hispanica ATCC 33960] |
| NP_988010.1 | superfamily II helicase [Methanococcus maripaludis S2] >emb\|CAF30446.1\| |
| | superfamily II helicase [Methanococcus maripaludis S2] |
| YP_565780.1 | ski2-like helicase [Methanococcoides burtonii DSM 6242] >sp\|Q12WZ6.1\|HELS_METBU |
| | RecName: Full = Putative ski2-type helicase >gb\|ABE52030.1\| |
| | DEAD/DEAH box helicase-like protein [Methanococcoides |
| | burtonii DSM 6242] |
| YP_001549808.1 | DEAD/DEAH box helicase domain-containing protein [Methanococcus |
| | maripaludis C6] >gb\|ABX02576.1\| DEAD/DEAH box helicase domain protein |
| | [Methanococcus maripaludis C6] |
| YP_001548609.1 | DEAD/DEAH box helicase domain-containing protein [Methanococcus |
| | maripaludis C6] >gb\|ABX01377.1\| DEAD/DEAH box helicase domain protein |
| | [Methanococcus maripaludis C6] |
| YP_001329359.1 | DEAD/DEAH box helicase domain-containing protein [Methanococcus |
| | maripaludis C7] >gb\|ABR65208.1\| DEAD/DEAH box helicase domain protein |
| | [Methanococcus maripaludis C7] |
| YP_004595982.1 | ski2-type helicase [Halopiger xanaduensis SH-6] >gb\|AEH36103.1\| ski2-type |
| | helicase [Halopiger xanaduensis SH-6] |
| YP_656795.1 | ski2-like helicase [Haloquadratum walsbyi DSM 16790] >emb\|CAJ51138.1\| |
| | ATP-dependent DNA helicase [Haloquadratum walsbyi DSM 16790] |
| CCC38992.1 | ATP-dependent DNA helicase Hel308 [Haloquadratum walsbyi C23] |
| YP_004035272.1 | superfamily ii helicase [Halogeometricum borinquense DSM |
| | 11551] >gb\|ADQ65833.1\| superfamily II helicase |
| | [Halogeometricum borinquense DSM 11551] |
| YP_137330.1 | ski2-like helicase [Haloarcula marismortui ATCC |
| | 43049] >sp\|Q5UYM9.1\|HELS_HALMA RecName: Full = Putative |
| | ski2-type helicase >gb\|AAV47624.1\| putative ski2-type helicase |
| | [Haloarcula marismortui ATCC 43049] |
| YP_001581577.1 | DEAD/DEAH box helicase domain-containing protein [Nitrosopumilus |
| | maritimus SCM1] >gb\|ABX12139.1\| DEAD/DEAH box helicase domain protein |
| | [Nitrosopumilus maritimus SCM1] |
| EET90255.1 | DEAD/DEAH box helicase domain protein [Candidates Micrarchaeum |
| | acidiphilum ARMAN-2] |
| NP_376477.1 | helicase [Sulfolobus tokodaii str. 7] >sp\|Q974S1.1\|HELS_SULTO RecName: |
| | Full = Putative ski2-type helicase >dbj\|BAK54341.1\| Holliday junction migration |
| | helicase [Sulfolobus tokodaii str. 7] |
| YP_001097792.1 | DEAD/DEAH box helicase domain-containing protein [Methanococcus |
| | maripaludis C5] >gb\|ABO35578.1\| DEAD/DEAH box helicase domain protein |
| | [Methanococcus maripaludis C5] |
| ZP_08667240.1 | DEAD/DEAH box helicase domain protein [Nitrosopumilus sp. |
| | MY1] >gb\|EGP92972.1\| DEAD/DEAH box helicase domain protein |
| | [Nitrosopumilus sp. MY1] |
| YP_254972.1 | DNA helicase [Sulfolobus acidocaldarius DSM 639] >sp\|Q4JC00.1\|HELS_SULAC |
| | RecName: Full = Putative ski2-type helicase >gb\|AAY79679.1\| |
| | DNA helicase [Sulfolobus acidocaldarius DSM 639] |
| EFD92533.1 | DEAD/DEAH box helicase domain protein [Candidates Parvarchaeum |
| | acidophilus ARMAN-5] |
| YP_003176527.1 | ski2-like helicase [Halomicrobium mukohataei DSM 12286] >gb\|ACV46820.1\| |
| | DEAD/DEAH box helicase domain protein [Halomicrobium mukohataei DSM |
| | 12286] |
| EGD71904.1 | DEAD/DEAH box helicase domain protein [Candidates Parvarchaeum |
| | acidophilus ARMAN-5_′5-way FS′] |
| YP_001040230.1 | DEAD/DEAH box helicase domain-containing protein [Staphylothermus marinus |
| | F1] >gb\|ABN69322.1\| DEAD/DEAH box helicase domain protein |
| | [Staphylothermus marinus F1] |
| ABZ07376.1 | putative DEAD/DEAH box helicase [uncultured marine crenarchaeote |
| | HF4000_ANIW133M9] |
| YP_001097458.1 | DEAD/DEAH box helicase domain-containing protein [Methanococcus |
| | maripaludis C5] >gb\|ABO35243.1\| DEAD/DEAH box helicase domain protein |
| | [Methanococcus maripaludis C5] |
| ABZ08606.1 | putative DEAD/DEAH box helicase [uncultured marine crenarchaeote |
| | HF4000_APKG3H9] |
| YP_325906.1 | ski2-like helicase [Natronomonas pharaonis DSM 2160] >sp\|Q3IU46.1\|HELS_NATPD |
| | RecName: Full = Putative ski2-type helicase >emb\|CAI48337.1\| |
| | ATP-dependent DNA helicase 1 [Natronomonas pharaonis DSM 2160] |
| YP_930665.1 | DEAD/DEAH box helicase domain-containing protein [Pyrobaculum islandicum |
| | DSM 4184] >gb\|ABL88322.1\| DEAD/DEAH box helicase domain protein |
| | [Pyrobaculum islandicum DSM 4184] |
| YP_001435870.1 | DEAD/DEAH box helicase [Ignicoccus hospitalis KIN4/I] >gb\|ABU82463.1\| |
| | DEAD/DEAH box helicase domain protein [Ignicoccus hospitalis KIN4/I] |
| YP_003668634.1 | DEAD/DEAH box helicase domain-containing protein [Staphylothermus |
| | hellenicus DSM 12710] >gb\|ADI31735.1\| DEAD/DEAH box helicase domain |
| | protein [Staphylothermus hellenicus DSM 12710] |
| ZP_08558598.1 | ski2-like helicase [Halorhabdus tiamatea SARL4B] >gb\|EGM36528.1\| ski2-like |
| | helicase [Halorhabdus tiamatea SARL4B] |
| YP_002428409.1 | DEAD/DEAH box helicase domain-containing protein [Desulfurococcus |
| | kamchatkensis 1221n] >gb\|ACL11042.1\| DEAD/DEAH box helicase domain |
| | protein [Desulfurococcus kamchatkensis 1221n] |
| YP_004336918.1 | ATP-dependent, DNA binding helicase [Thermoproteus uzoniensis |
| | 768-20] >gb\|AEA11606.1\| ATP-dependent, DNA binding helicase |
| | [Thermoproteus uzoniensis 768-20] |
| ZP_08257442.1 | DEAD/DEAH box helicase domain-containing protein [Candidatus |
| | Nitrosoarchaeum limnia SFB1] >gb\|EGG41989.1\| DEAD/DEAH box helicase |
| | domain-containing protein [Candidatus Nitrosoarchaeum limnia SFB1] |
| YP_004459284.1 | DEAD/DEAH box helicase domain-containing protein [Acidianus hospitalis |
| | W1] >gb\|AEE94986.1\| DEAD/DEAH box helicase domain protein |
| | [Acidianus hospitalis W1] |
| NP_558924.1 | ATP-dependent, DNA binding helicase [Pyrobaculum aerophilum str. |
| | IM2] >gb\|AAL63106.1\| ATP-dependent, DNA binding helicase |
| | [Pyrobaculum aerophilum str. IM2] |
| YP_004409449.1 | DEAD/DEAH box helicase domain-containing protein [Metallosphaera cuprina |
| | Ar-4] >gb\|AEB94965.1\| DEAD/DEAH box helicase domain-containing protein |
| | [Metallosphaera cuprina Ar-4] |
| YP_003649556.1 | DEAD/DEAH box helicase domain-containing protein [Thermosphaera |
| | aggregans DSM 11486] >gb\|ADG90604.1\| DEAD/DEAH box helicase domain |
| | protein [Thermosphaera aggregans DSM 11486] |
| ZP_06387115.1 | DEAD/DEAH box helicase domain protein [Sulfolobus solfataricus |
| | 98/2] >gb\|ACX90562.1\| DEAD/DEAH box helicase domain protein |
| | [Sulfolobus solfataricus 98/2] |
| 2VA8_A | Chain A, Dna Repair Helicase Hel308 >pdb\|2VA8\|B Chain B, Dna Repair |
| | Helicase Hel308 >emb\|CAO85626.1\| DNA helicase [Sulfolobus solfataricus] |
| YP_004809267.1 | ski2-type helicase [Halophilic archaeon DL31] >gb\|AEN06894.1\| ski2-type |
| | helicase [Halophilic archaeon DL31] |
| ADX84345.1 | DEAD/DEAH box helicase domain protein [Sulfolobus islandicus |
| | REY15A] >gb\|ADX81629.1\| DEAD/DEAH box helicase domain protein |
| | [Sulfolobus islandicus HVE10/4] |
| YP_002828439.1 | DEAD/DEAH box helicase [Sulfolobus islandicus M.14.25] >ref\|YP_002842325.1\| |
| | DEAD/DEAH box helicase domain protein [Sulfolobus islandicus |
| | M.16.27] >gb\|ACP37141.1\| DEAD/DEAH box helicase domain protein |
| | [Sulfolobus islandicus M.14.25] >gb\|ACP54280.1\| DEAD/DEAH box helicase |
| | domain protein [Sulfolobus islandicus M.16.27] |
| YP_002913571.1 | DEAD/DEAH box helicase domain protein [Sulfolobus islandicus |
| | M.16.4] >gb\|ACR40903.1\| DEAD/DEAH box helicase domain protein |
| | [Sulfolobus islandicus M.16.4] |
| Q97VY9.1 | RecName: Full = Putative ski2-type helicase |
| YP_002841682.1 | DEAD/DEAH box helicase domain protein [Sulfolobus islandicus |
| | Y.N.15.51] >gb\|ACP49760.1\| DEAD/DEAH box helicase domain protein |
| | [Sulfolobus islandicus Y.N.15.51] |
| YP_002831080.1 | DEAD/DEAH box helicase domain protein [Sulfolobus islandicus |
| | L.S.2.15] >ref\|YP_003418425.1\| DEAD/DEAH box helicase domain protein |
| | [Sulfolobus islandicus L.D.8.5] >gb\|ACP34435.1\| DEAD/DEAH box |
| | helicase domain protein [Sulfolobus islandicus L.S.2.15] >gb\|ADB86055.1\| |
| | DEAD/DEAH box helicase domain protein [Sulfolobus islandicus L.D.8.5] |
| YP_001054984.1 | DEAD/DEAH box helicase domain-containing protein [Pyrobaculum calidifontis |
| | JCM 11548] >sp\|A3MSA1.1\|HELS_PYRCJ RecName: Full = Putative ski2-type |
| | helicase >gb\|ABO07518.1\| DEAD/DEAH box helicase domain protein |
| | [Pyrobaculum calidifontis JCM 11548] |
| NP_343811.1 | DNA helicase related protein [Sulfolobus solfataricus P2] >ref\|YP_002836469.1\| |
| | DEAD/DEAH box helicase [Sulfolobus islandicus Y.G.57.14] >gb\|AAK42601.1\| |
| | DNA helicase related protein [Sulfolobus solfataricus P2] >gb\|ACP44547.1\| |
| | DEAD/DEAH box helicase domain protein [Sulfolobus islandicus Y.G.57.14] |
| YP_001152379.1 | DEAD/DEAH box helicase domain-containing protein [Pyrobaculum arsenaticum |
| | DSM 13514] >gb\|ABP49727.1\| DEAD/DEAH box helicase domain protein |
| | [Pyrobaculum arsenaticum DSM 13514] |
| YP_001191456.1 | DEAD/DEAH box helicase domain-containing protein [Metallosphaera sedula |
| | DSM 5348] >gb\|ABP95532.1\| DEAD/DEAH box helicase domain protein |
| | [Metallosphaera sedula DSM 5348] |
| NP_147034.2 | holliday junction migration helicase [Aeropyrum pernix |
| | K1] >sp\|Q9YFQ8.2\|HELS_AERPE RecName: Full = Putative |
| | ski2-type helicase >dbj\|BAA79103.2\| holliday junction migration |
| | helicase [Aeropyrum pernix K1] |
| YP_024158.1 | ski2-like helicase [Picrophilus torridus DSM 9790] >gb\|AAT43965.1\| helicase |
| | involved in UV-protection [Picrophilus torridus DSM 9790] |
| YP_003816358.1 | Putative ski2-type helicase [Acidilobus saccharovorans 345-15] >gb\|ADL19327.1\| |
| | Putative ski2-type helicase [Acidilobus saccharovorans 345-15] |
| YP_003860265.1 | DEAD/DEAH box helicase domain protein [Ignisphaera aggregans DSM |
| | 17230] >gb\|ADM28385.1\| DEAD/DEAH box helicase domain protein |
| | [Ignisphaera aggregans DSM 17230] |
| NP_394295.1 | ski2-like helicase [Thermoplasma acidophilum DSM |
| | 1728] >sp\|Q9HJX7.1\|HELS_THEAC RecName: Full = Putative ski2-type |
| | helicase >emb\|CAC11964.1\| DNA helicase related protein |
| | [Thermoplasma acidophilum] |
| YP_876638.1 | superfamily II helicase [Cenarchaeum symbiosum A] >gb\|ABK78334.1\| |
| | superfamily II helicase [Cenarchaeum symbiosum A] |
| ZP_05571398.1 | ski2-like helicase [Ferroplasma acidarmanus fer1] |
| YP_004176252.1 | DEAD/DEAH box helicase domain-containing protein [Desulfurococcus mucosus |
| | DSM 2162] >gb\|ADV64770.1\| DEAD/DEAH box helicase domain protein |
| | [Desulfurococcus mucosus DSM 2162] |
| YP_001737782.1 | DEAD/DEAH box helicase domain-containing protein [Candidatus Korarchaeum |
| | cryptofilum OPF8] >gb\|ACB08099.1\| DEAD/DEAH box helicase domain protein |
| | [Candidatus Korarchaeum cryptofilum OPF8] |
| EGQ40435.1 | superfamily II helicase [Candidatus Nanosalinarum sp. J07AB56] |
| YP_002567343.1 | ski2-like helicase [Halorubrum lacusprofundi ATCC 49239] >gb\|ACM58273.1\| |
| | DEAD/DEAH box helicase domain protein [Halorubrum lacusprofundi ATCC |
| | 49239] |
| YP_001793507.1 | DEAD/DEAH box helicase domain-containing protein [Thermoproteus |
| | neutrophilus V24Sta] >gb\|ACB39061.1\| DEAD/DEAH box helicase domain |
| | protein [Thermoproteus neutrophilus V24Sta] |
| YP_003534088.1 | ATP-dependent DNA helicase Hel308a [Haloferax volcanii |
| | DS2] >gb\|ADE04048.1\| ATP-dependent DNA helicase Hel308a |
| | [Haloferax volcanii DS2] |
| YP_004037165.1 | superfamily ii helicase [Halogeometricum borinquense DSM |
| | 11551] >gb\|ADQ67720.1\| superfamily II helicase |
| | [Halogeometricum borinquense DSM 11551] |
| NP_111333.1 | ski2-like helicase [Thermoplasma volcanium |
| | GSS1] >sp\|Q97AI2.1\|HELS_THEVO RecName: Full = |
| | Putative ski2-type helicase >dbj\|BAB59970.1\| DNA |
| | helicase [Thermoplasma volcanium GSS1] |
| YP_002565871.1 | DEAD/DEAH box helicase [Halorubrum lacusprofundi ATCC |
| | 49239] >gb\|ACM56801.1\| DEAD/DEAH box helicase domain |
| | protein [Halorubrum lacusprofundi ATCC 49239] |
| CCC39675.1 | ATP-dependent DNA helicase Hel308 [Haloquadratum walsbyi C23] |
| YP_657401.1 | ATP-dependent DNA helicase [Haloquadratum walsbyi DSM |
| | 16790] >emb\|CAJ51759.1\| ATP-dependent DNA helicase |
| | [Haloquadratum walsbyi DSM 16790] |
| YP_003535028.1 | ATP-dependent DNA helicase Hel308b [Haloferax volcanii |
| | DS2] >gb\|ADE02398.1\| ATP-dependent DNA helicase Hel308b |
| | [Haloferax volcanii DS2] |
| YP_003706863.1 | DEAD/DEAH box helicase domain-containing protein [Methanococcus voltae |
| | A3] >gb\|ADI35890.1\| DEAD/DEAH box helicase domain protein |
| | [Methanococcus voltae A3] |
| ABD17736.1 | helicase [Methanococcus voltae PS] |
| NP_613398.1 | superfamily II helicase [Methanopyrus kandleri AV19] >gb\|AAM01328.1\| |
| | Predicted Superfamily II helicase [Methanopyrus kandleri AV19] |
| CBH38575.1 | putative ski2-type helicase [uncultured archaeon] |
| EEZ93258.1 | DEAD/DEAH box helicase domain protein [Candidates Parvarchaeum |
| | acidiphilum ARMAN-4] |
| EGQ40350.1 | superfamily II helicase [Candidates Nanosalinarum sp. J07AB56] |
| YP_004004246.1 | dead/deah box helicase domain protein [Methanothermus fervidus DSM |
| | 2088] >gb\|ADP77484.1\| DEAD/DEAH box helicase domain protein |
| | [Methanothermus fervidus DSM 2088] |
| YP_003850109.1 | helicase [Methanothermobacter marburgensis str. Marburg] >gb\|ADL58796.1\| |
| | predicted helicase [Methanothermobacter marburgensis str. Marburg] |
| YP_003424423.1 | DEAD/DEAH box helicase domain-containing protein [Methanobrevibacter |
| | ruminantium M1] >gb\|ADC47531.1\| DEAD/DEAH box helicase domain- |
| | containing protein [Methanobrevibacter ruminantium M1] |
| YP_004291107.1 | DEAD/DEAH box helicase domain-containing protein [Methanobacterium sp. |
| | AL-21] >gb\|ADZ10135.1\| DEAD/DEAH box helicase domain protein |
| | [Methanobacterium sp. AL-21] |
| YP_447162.1 | helicase [Methanosphaera stadtmanae DSM 3091] >gb\|ABC56519.1\| predicted |
| | helicase [Methanosphaera stadtmanae DSM 3091] |
| YP_004519549.1 | DEAD/DEAH box helicase domain-containing protein [Methanobacterium sp. |
| | SWAN-1] >gb\|AEG17748.1\| DEAD/DEAH box helicase domain protein |
| | [Methanobacterium sp. SWAN-1] |
| NP_275949.1 | DNA helicase related protein [Methanothermobacter thermautotrophicus str. |
| | Delta H] >sp\|O26901.1\|HELS_METTH RecName: Full = Putative ski2-type |
| | helicase >gb\|AAB85310.1\| DNA helicase related protein [Methanothermobacter |
| | thermautotrophicus str. Delta H] |
| ZP_05975717.2 | putative Ski2-type helicase [Methanobrevibacter smithii DSM |
| | 2374] >gb\|EFC93382.1\| putative Ski2-type helicase |
| | [Methanobrevibacter smithii DSM 2374] |
| ZP_03607647.1 | hypothetical protein METSMIALI_00751 [Methanobrevibacter smithii DSM |
| | 2375] >gb\|EEE41862.1\| hypothetical protein METSMIALI_00751 |
| | [Methanobrevibacter smithii DSM 2375] |
| YP_001273412.1 | ATP-dependent helicase [Methanobrevibacter smithii ATCC |
| | 35061] >gb\|ABQ87044.1\| ATP-dependent helicase |
| | [Methanobrevibacter smithii ATCC 35061] |
| YP_003247505.1 | DEAD/DEAH box helicase domain protein [Methanocaldococcus vulcanius |
| | M7] >gb\|ACX73023.1\| DEAD/DEAH box helicase domain protein |
| | [Methanocaldococcus vulcanius M7] |
| NP_248116.1 | SKI2 family helicase [Methanocaldococcus jannaschii DSM |
| | 2661] >sp\|Q58524.1\|HELS_METJA RecName: Full = Putative |
| | ski2-type helicase; Contains: RecName: Full = Endonuclease PI-MjaHel; |
| | AltName: Full = Mja Hel intein; AltName: Full = Mja Pep3 intein >gb\|AAB99126.1\| |
| | putative SKI2-family helicase [Methanocaldococcus jannaschii DSM 2661] |
| YP_001324295.1 | DEAD/DEAH box helicase domain-containing protein [Methanococcus aeolicus |
| | Nankai-3] >gb\|ABR55683.1\| DEAD/DEAH box helicase domain protein |
| | [Methanococcus aeolicus Nankai-3] |
| YP_003536960.1 | Pre-mRNA splicing helicase [Haloferax volcanii DS2] >gb\|ADE02332.1\| Pre- |
| | mRNA splicing helicase [Haloferax volcanii DS2] |
| YP_003131029.1 | DEAD/DEAH box helicase domain protein [Halorhabdus utahensis DSM |
| | 12940] >gb\|ACV12296.1\| DEAD/DEAH box helicase domain protein |
| | [Halorhabdus utahensis DSM 12940] |
| YP_002567151.1 | DEAD/DEAH box helicase [Halorubrum lacusprofundi ATCC |
| | 49239] >gb\|ACM58081.1\| DEAD/DEAH box helicase domain protein |
| | [Halorubrum lacusprofundi ATCC 49239] |
| YP_004035351.1 | superfamily ii helicase [Halogeometricum borinquense DSM |
| | 11551] >gb\|ADQ65912.1\| superfamily II helicase |
| | [Halogeometricum borinquense DSM 11551] |
| YP_004808851.1 | DEAD/DEAH box helicase domain-containing protein [Halophilic archaeon |
| | DL31] >gb\|AEN06478.1\| DEAD/DEAH box helicase domain protein [Halophilic |
| | archaeon DL31] |
| XP_002716686.1 | PREDICTED: DNA polymerase theta isoform 1 [Oryctolagus cuniculus] |
| YP_656834.1 | ATP-dependent DNA helicase [Haloquadratum walsbyi DSM |
| | 16790] >emb\|CAJ51176.1\| ATP-dependent DNA helicase |
| | [Haloquadratum walsbyi DSM 16790] |
| XP_003248103.1 | PREDICTED: DNA polymerase theta-like isoform 1 [Acyrthosiphon pisum] |
| ABC72356.1 | ATP-dependent DNA helicase [Haloquadratum walsbyi] |
| CCC39031.1 | DEAD/DEAH box helicase [Haloquadratum walsbyi C23] |
| XP_001165150.2 | PREDICTED: DNA polymerase theta isoform 1 [Pan troglodytes] |
| XP_003225852.1 | PREDICTED: DNA polymerase theta-like [Anolis carolinensis] |
| XP_615375.3 | PREDICTED: DNA polymerase theta [Bos taurus] >ref\|XP_002684835.1\| |
| | PREDICTED: polymerase (DNA directed), theta-like [Bos |
| | taurus] >gb\|DAA33456.1\| polymerase (DNA directed), |
| | theta-like [Bos taurus] |
| XP_002813286.1 | PREDICTED: LOW QUALITY PROTEIN: DNA polymerase theta-like [Pongo |
| | abelii] |
| AAR08421.2 | DNA polymerase theta [Homo sapiens] |
| EAW79510.1 | polymerase (DNA directed), theta, isoform CRA_a [Homo sapiens] |
| NP_955452.3 | DNA polymerase theta [Homo sapiens] >sp\|O75417.2\|DPOLQ_HUMAN |
| | RecName: Full = DNA polymerase theta; AltName: Full = DNA polymerase |
| | eta >gb\|AAI72289.1\| Polymerase (DNA directed), theta |
| | [synthetic polynucleotide] |
| NP_001099348.1 | DNA polymerase theta [Rattus norvegicus] >gb\|EDM11249.1\| polymerase (DNA |
| | directed), theta (predicted), isoform CRA_a [Rattus norvegicus] |
| XP_003341262.1 | PREDICTED: LOW QUALITY PROTEIN: DNA polymerase theta-like |
| | [Monodelphis domestica] |
| XP_001502374.3 | PREDICTED: DNA polymerase theta [Equus caballus] |
| XP_545125.3 | PREDICTED: LOW QUALITY PROTEIN: DNA polymerase theta [Canis lupus |
| | familiaris] |
| XP_002928855.1 | PREDICTED: LOW QUALITY PROTEIN: DNA polymerase theta-like |
| | [Ailuropoda melanoleuca] |
| NP_084253.1 | DNA polymerase theta isoform 1 [Mus musculus] >gb\|AAL77225.1\| DNA |
| | polymerase theta [Mus musculus] >gb\|EDK97951.1\| polymerase (DNA directed), |
| | theta, isoform CRA_a [Mus musculus] >gb\|AAI38361.1\| Polymerase (DNA |
| | directed), theta [Mus musculus] >gb\|AAI57901.1\| Polymerase (DNA directed), |
| | theta [Mus musculus] |
| AAK39635.1 | DNA polymerase theta [Homo sapiens] |
| AAN39838.1 | DNA polymerase Q [Mus musculus] |
| XP_003412882.1 | PREDICTED: DNA polymerase theta [Loxodonta africana] |
| YP_003735206.1 | DEAD/DEAH box helicase domain-containing protein [Halalkalicoccus jeotgali |
| | B3] >gb\|ADJ13414.1\| DEAD/DEAH box helicase domain protein |
| | [Halalkalicoccus jeotgali B3] |
| YP_004794841.1 | pre-mRNA splicing helicase [Haloarcula hispanica ATCC |
| | 33960] >gb\|AEM55853.1\| pre-mRNA splicing helicase [Haloarcula |
| | hispanica ATCC 33960] |
| XP_416549.2 | PREDICTED: similar to DNA polymerase theta [Gallus gallus] |
| XP_003427319.1 | PREDICTED: helicase POLQ-like isoform 2 [Nasonia vitripennis] |
| XP_003202748.1 | PREDICTED: DNA polymerase theta-like [Meleagris gallopavo] |
| XP_969311.1 | PREDICTED: similar to DNA polymerase theta [Tribolium |
| | castaneum] >gb\|EEZ97532.1\| hypothetical protein |
| | TcasGA2_TC011380 [Tribolium castaneum] |
| ZP_08046037.1 | DEAD/DEAH box helicase domain protein [Haladaptatus paucihalophilus |
| | DX253] >gb\|EFW90685.1\| DEAD/DEAH box helicase domain protein |
| | [Haladaptatus paucihalophilus DX253] |
| YP_461714.1 | helicase [Syntrophus aciditrophicus SB] >gb\|ABC77546.1\| helicase |
| | [Syntrophus aciditrophicus SB] |
| YP_003176510.1 | DEAD/DEAH box helicase [Halomicrobium mukohataei DSM |
| | 12286] >gb\|ACV46803.1\| DEAD/DEAH box helicase domain protein |
| | [Halomicrobium mukohataei DSM 12286] |
| YP_137400.1 | Pre-mRNA splicing helicase [Haloarcula marismortui ATCC |
| | 43049] >gb\|AAV47694.1\| Pre-mRNA splicing helicase |
| | [Haloarcula marismortui ATCC 43049] |
| NP_001184156.1 | polymerase (DNA directed), theta [Xenopus (Silurana) tropicalis] |
| NP_280861.1 | pre-mRNA splicing helicase [Halobacterium sp. NRC-1] >ref\|YP_001689987.1\| |
| | ATP-dependent DNA helicase [Halobacterium salinarum R1] >gb\|AAG20341.1\| |
| | pre-mRNA splicing helicase [Halobacterium sp. NRC-1] >emb\|CAP14641.1\| |
| | ATP-dependent DNA helicase [Halobacterium salinarum R1] |
| YP_004595640.1 | DEAD/DEAH box helicase domain-containing protein [Halopiger xanaduensis |
| | SH-6] >gb\|AEH35761.1\| DEAD/DEAH box helicase domain protein [Halopiger |
| | xanaduensis SH-6] |
| XP_001521144.2 | PREDICTED: DNA polymerase theta, partial [Ornithorhynchus anatinus] |
| XP_003261953.1 | PREDICTED: DNA polymerase theta, partial [Nomascus leucogenys] |
| XP_001358456.2 | GA19301 [Drosophila pseudoobscura pseudoobscura] >gb\|EAL27595.2\| |
| | GA19301 [Drosophila pseudoobscura pseudoobscura] |
| ZP_08560003.1 | DEAD/DEAH box helicase domain protein [Halorhabdus tiamatea |
| | SARL4B] >gb\|EGM34502.1\| DEAD/DEAH box helicase domain protein |
| | [Halorhabdus tiamatea SARL4B] |
| XP_002187783.1 | PREDICTED: similar to polymerase (DNA directed), theta [Taeniopygia guttata] |
| XP_002112587.1 | hypothetical protein TRIADDRAFT_25163 [Trichoplax |
| | adhaerens] >gb\|EDV24697.1\| hypothetical protein TRIADDRAFT_25163 |
| | [Trichoplax adhaerens] |
| YP_003405139.1 | DEAD/DEAH box helicase [Haloterrigena turkmenica DSM |
| | 5511] >gb\|ADB62466.1\| DEAD/DEAH box helicase domain protein |
| | [Haloterrigena turkmenica DSM 5511] |
| EGV92665.1 | DNA polymerase theta [Cricetulus griseus] |
| CBY24305.1 | unnamed protein product [Oikopleura dioica] |
| YP_003130565.1 | DEAD/DEAH box helicase domain protein [Halorhabdus utahensis DSM |
| | 12940] >gb\|ACV11832.1\| DEAD/DEAH box helicase domain protein |
| | [Halorhabdus utahensis DSM 12940] |
| YP_003479811.1 | DEAD/DEAH box helicase [Natrialba magadii ATCC 43099] >gb\|ADD05249.1\| |
| | DEAD/DEAH box helicase domain protein [Natrialba magadii ATCC 43099] |
| EFB22383.1 | hypothetical protein PANDA_000253 [Ailuropoda melanoleuca] |
| YP_003357334.1 | putative ATP-dependent helicase [Methanocella paludicola |
| | SANAE] >dbj\|BAI62351.1\| putative ATP-dependent helicase |
| | [Methanocella paludicola SANAE] |
| YP_325942.1 | ATP-dependent DNA helicase 2 [Natronomonas pharaonis DSM |
| | 2160] >emb\|CAI48373.2\| ATP-dependent DNA helicase 2 |
| | [Natronomonas pharaonis DSM 2160] |
| XP_002912509.1 | PREDICTED: LOW QUALITY PROTEIN: helicase POLQ-like [Ailuropoda |
| | melanoleuca] |
| XP_002704678.1 | PREDICTED: helicase, POLQ-like [Bos taurus] |
| CAE47762.2 | novel protein similar to humna DNA-directed polymerase theta (POLQ) [Danio |
| | rerio] |
| XP_003205636.1 | PREDICTED: helicase POLQ-like [Meleagris gallopavo] |
| XP_544959.2 | PREDICTED: helicase, POLQ-like [Canis lupus familiaris] |
| EFX86757.1 | hypothetical protein DAPPUDRAFT_312857 [Daphnia pulex] |
| YP_003389641.1 | DEAD/DEAH box helicase [Spirosoma linguale DSM 74] >gb\|ADB40842.1\| |
| | DEAD/DEAH box helicase domain protein [Spirosoma linguale DSM 74] |
| XP_002602932.1 | hypothetical protein BRAFLDRAFT_251779 [Branchiostoma |
| | floridae] >gb\|EEN58944.1\| hypothetical protein BRAFLDRAFT_251779 |
| | [Branchiostoma floridae] |
| YP_004144962.1 | peptidase C14 caspase catalytic subunit p20 [Mesorhizobium ciceri biovar |
| | biserrulae WSM1271] >ref\|YP_004614892.1\| DEAD/DEAH box helicase |
| | domain-containing protein [Mesorhizobium opportunistum |
| | WSM2075] >gb\|ADV14912.1\| peptidase C14 caspase catalytic subunit p20 |
| | [Mesorhizobium ciceri biovar biserrulae WSM1271] >gb\|AEH90798.1\| |
| | DEAD/DEAH box helicase domain protein [Mesorhizobium opportunistum WSM2075] |
| XP_002124758.1 | PREDICTED: similar to DNA polymerase theta [Ciona intestinalis] |
| XP_694437.5 | PREDICTED: DNA polymerase theta [Danio rerio] |
| XP_420565.1 | PREDICTED: similar to DNA helicase HEL308 [Gallus gallus] |
| XP_003129397.1 | PREDICTED: helicase POLQ-like [Sus scrofa] |
| EDL20278.1 | mCG128467, isoform CRA_b [Mus musculus] |
| XP_001517710.2 | PREDICTED: helicase POLQ, partial [Ornithorhynchus anatinus] |
| AAH82601.1 | Helicase, mus308-like (Drosophila) [Mus musculus] |
| XP_003384429.1 | PREDICTED: DNA polymerase theta-like [Amphimedon queenslandica] |
| XP_003221282.1 | PREDICTED: helicase POLQ-like [Anolis carolinensis] |
| NP_524333.1 | mutagen-sensitive 308 [Drosophila melanogaster] >gb\|AAB67306.1\| Mus308 |
| | [Drosophila melanogaster] >gb\|AAF54858.1\| mutagen-sensitive 308 [Drosophila |
| | melanogaster] >gb\|ACH92234.1\| FI03732p [Drosophila melanogaster] |
| AAX33507.1 | LP14642p [Drosophila melanogaster] |
| NP_001074576.1 | helicase POLQ-like [Mus musculus] >sp\|Q2VPA6.2\|HELQ_MOUSE RecName: |
| | Full = Helicase POLQ-like; AltName: Full = Mus308-like helicase; AltName: |
| | Full = POLQ-like helicase >gb\|AAI09171.2\| Helicase, mus308-like (Drosophila) |
| | [Mus musculus] |
| YP_003523727.1 | DEAD/DEAH box helicase domain protein [Sideroxydans lithotrophicus |
| | ES-1] >gb\|ADE11340.1\| DEAD/DEAH box helicase domain protein [Sideroxydans |
| | lithotrophicus ES-1] |
| XP_002120889.1 | PREDICTED: similar to DNA helicase HEL308 [Ciona intestinalis] |
| XP_001892566.1 | Type III restriction enzyme, res subunit family protein [Brugia |
| | malayi] >gb\|EDP38603.1\| Type III restriction enzyme, res subunit family |
| | protein [Brugia malayi] |
| ABZ09232.1 | putative helicase conserved C-terminal domain protein [uncultured marine |
| | crenarchaeote HF4000_APKG7F11] |
| XP_002814981.1 | PREDICTED: LOW QUALITY PROTEIN: helicase POLQ-like [Pongo abelii] |
| XP_002717082.1 | PREDICTED: DNA helicase HEL308 [Oryctolagus cuniculus] |
| XP_001104832.1 | PREDICTED: helicase, POLQ-like [Macaca mulatta] |
| AAL85274.1 | DNA helicase HEL308 [Homo sapiens] |
| NP_598375.2 | helicase POLQ-like [Homo sapiens] >gb\|EAX05934.1\| DNA helicase HEL308, |
| | isoform CRA_a [Homo sapiens] >gb\|AAI41525.1\| Helicase, POLQ-like |
| | [synthetic polynucleotide] |
| Q8TDG4.2 | RecName: Full = Helicase POLQ-like; AltName: Full = Mus308-like helicase; |
| | AltName: Full = POLQ-like helicase |
| XP_003265889.1 | PREDICTED: helicase POLQ [Nomascus leucogenys] |
| XP_002745688.1 | PREDICTED: helicase POLQ-like [Callithrix jacchus] |
| XP_003310356.1 | PREDICTED: LOW QUALITY PROTEIN: helicase POLQ-like [Pan |
| | troglodytes] |
| NP_001014156.2 | helicase, POLQ-like [Rattus norvegicus] >ref\|XP_001060858.1\| PREDICTED: |
| | helicase, POLQ-like [Rattus norvegicus] >gb\|EDL99554.1\| rCG37823, isoform |
| | CRA_c [Rattus norvegicus] |
| XP_001850567.1 | ATP-dependent DNA helicase MER3 [Culex quinquefasciatus] >gb\|EDS32308.1\| |
| | ATP-dependent DNA helicase MER3 [Culex quinquefasciatus] |
| XP_003427318.1 | PREDICTED: helicase POLQ-like isoform 1 [Nasonia vitripennis] |
| XP_003143912.1 | hypothetical protein LOAG_08332 [Loa loa] >gb\|EFO20157.1\| hypothetical |
| | protein LOAG_08332 [Loa loa] |
| CAG11187.1 | unnamed protein product [Tetraodon nigroviridis] |
| XP_001111254.2 | PREDICTED: DNA polymerase theta isoform 2 [Macaca mulatta] |
| XP_003414242.1 | PREDICTED: helicase POLQ [Loxodonta africana] |
| XP_002681870.1 | predicted protein [Naegleria gruberi] >gb\|EFC49126.1\| predicted protein |
| | [Naegleria gruberi] |
| EAX05935.1 | DNA helicase HEL308, isoform CRA_b [Homo sapiens] |
| AAH59917.1 | Ascc3 protein [Mus musculus] |
| ZP_07082808.1 | DEAD/DEAH box helicase domain protein [Sphingobacterium spiritivorum |
| | ATCC 33861] >gb\|EFK55937.1\| DEAD/DEAH box helicase domain protein |
| | [Sphingobacterium spiritivorum ATCC 33861] |
| XP_001494572.3 | PREDICTED: LOW QUALITY PROTEIN: helicase POLQ-like [Equus caballus] |
| XP_002714920.1 | PREDICTED: activating signal cointegrator 1 complex subunit 3 [Oryctolagus |
| | cuniculus] |
| XP_002598278.1 | hypothetical protein BRAFLDRAFT_204526 [Branchiostoma |
| | floridae] >gb\|EEN54290.1\| hypothetical protein BRAFLDRAFT_204526 |
| | [Branchiostoma floridae] |
| XP_001943294.1 | PREDICTED: helicase POLQ-like isoform 1 [Acyrthosiphon |
| | pisum] >ref\|XP_003240510.1\| PREDICTED: helicase POLQ-like isoform 2 |
| | [Acyrthosiphon pisum] |
| XP_002803889.1 | PREDICTED: activating signal cointegrator 1 complex subunit 3-like [Macaca |
| | mulatta] |
| XP_001651546.1 | DNA polymerase theta [Aedes aegypti] >gb\|EAT42599.1\| DNA polymerase theta |
| | [Aedes aegypti] |
| CAA11679.1 | RNA helicase [Homo sapiens] |
| XP_002837795.1 | hypothetical protein [Tuber melanosporum Mel28] >emb\|CAZ81986.1\| unnamed |
| | protein product [Tuber melanosporum] |
| EGT47882.1 | hypothetical protein CAEBREN_02542 [Caenorhabditis brenneri] |
| EDL99655.1 | activating signal cointegrator 1 complex subunit 3 (predicted), isoform CRA_b |
| | [Rattus norvegicus] |
| NP_932124.2 | activating signal cointegrator 1 complex subunit 3 [Mus musculus] |
| EDL05054.1 | mCG119534 [Mus musculus] |
| gi\|352115865 | DEAD/DEAH box helicase domain protein |
| ZP_08963952.1 | [Natrinema pellirubrum DSM 15624] |

The Hel308 helicase is more preferably one of the helicases shown in Table 5 below or a variant thereof. The Hel308 helicase more preferably comprises the sequence of one of the helicases shown in Table 5, i.e. one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58, or a variant thereof.

TABLE 5
More preferred Hel308 helicases and most preferred Hel308
motifs and extended Hel308 motifs

| | | | % | % | | |
|---|---|---|---|---|---|---|
| SEQ | | | Identity | Identity | | |
| ID | | | Hel308 | Hel308 | Hel308 | Extended Hel308 |
| NO: | Helicase | Names | Pfu | Mbu | motif | motif |
| 10 | Hel308 Mbu | Methanococcoides | 37% | — | QMAGRAGR | QMAGRAGRP |
| | | burtonii | | | (SEQ ID NO: | (SEQ ID NO: 12) |
| | | | | | 11) | |
| 13 | Hel308 Pfu | Pyrococcus | — | 37% | QMLGRAGR | QMLGRAGRP |
| | | furiosus DSM | | | (SEQ ID NO: | (SEQ ID NO: 15) |
| | | 3638 | | | 14) | |
| 16 | Hel308 Hvo | Haloferax | 34% | 41% | QMMGRAGR | QMMGRAGRP |
| | | volcanii | | | (SEQ ID NO: | (SEQ ID NO: 18) |
| | | | | | 17) | |
| 19 | Hel308 Hla | Halorubrum | 35% | 42% | QMCGRAGR | QMCGRAGRP |
| | | lacusprofundi | | | (SEQ ID NO: | (SEQ ID NO: 21) |
| | | | | | 20) | |
| 22 | Hel308 Csy | Cenarchaeum | 34% | 34% | QLCGRAGR | QLCGRAGRP |
| | | symbiosum | | | (SEQ ID NO: | (SEQ ID NO: 24) |
| | | | | | 23) | |
| 25 | Hel308 Sso | Sulfolobus | 35% | 33% | QMSGRAGR | QMSGRAGRP |
| | | solfataricus | | | (SEQ ID NO: | (SEQ ID NO: 27) |
| | | | | | 26) | |
| 28 | Hel308 Mfr | Methanogenium | 37% | 44% | QMAGRAGR | QMAGRAGRP |
| | | frigidum | | | (SEQ ID NO: | (SEQ ID NO: 12) |
| | | | | | 11) | |
| 29 | Hel308 Mok | Methanothermococcus | 37% | 34% | QCIGRAGR | QCIGRAGRP |
| | | okinawensis | | | (SEQ ID NO: | (SEQ ID NO: 31) |
| | | | | | 30) | |
| 32 | Hel308 Mig | Methanotorris | 40% | 35% | QCIGRAGR | QCIGRAGRP |
| | | igneus Kol 5 | | | (SEQ ID NO: | (SEQ ID NO: 31) |
| | | | | | 30) | |
| 33 | Hel308 Tga | Thermococcus | 60% | 38% | QMMGRAGR | QMMGRAGRP |
| | | gammatolerans | | | (SEQ ID NO: | (SEQ ID NO: 18) |
| | | EJ3 | | | 17) | |
| 34 | Hel308 Tba | Thermococcus | 57% | 35% | QMIGRAGR | QMIGRAGRP |
| | | barophilus MP | | | (SEQ ID NO: | (SEQ ID NO: 36) |
| | | | | | 35) | |
| 37 | Hel308 Tsi | Thermococcus | 56% | 35% | QMMGRAGR | QMMGRAGRP |
| | | sibmcus MM 739 | | | (SEQ ID NO: | (SEQ ID NO: 18) |
| | | | | | 17) | |
| 38 | Hel308 Mba | Methanosarcina | 39% | 60% | QMAGRAGR | QMAGRAGRP |
| | | barkeri str. | | | (SEQ ID NO: | (SEQ ID NO: 12) |
| | | Fusaro | | | 11) | |
| 39 | Hel308 Mac | Methanosarcina | 38% | 60% | QMAGRAGR | QMAGRAGRP |
| | | acetivorans | | | (SEQ ID NO: | (SEQ ID NO: 12) |
| | | | | | 11) | |
| 40 | Hel308 | Methanohalophilus | 38% | 60% | QMAGRAGR | QMAGRAGRP |
| | Mmah | mahii DSM 5219 | | | (SEQ ID NO: | (SEQ ID NO: 12) |
| | | | | | 11) | |
| 41 | Hel308 | Methanosarcina | 38% | 60% | QMAGRAGR | QMAGRAGRP |
| | Mmaz | mazei | | | (SEQ ID NO: | (SEQ ID NO: 12) |
| | | | | | 11) | |
| 42 | Hel308 Mth | Methanosaeta | 39% | 46% | QMAGRAGR | QMAGRAGRP |
| | | thermophila PT | | | (SEQ ID NO: | (SEQ ID NO: 12) |
| | | | | | 11) | |
| 43 | Hel308 Mzh | Methanosalsum | 39% | 57% | QMAGRAGR | QMAGRAGRP |
| | | zhilinae DSM | | | (SEQ ID NO: | (SEQ ID NO: 12) |
| | | 4017 | | | 11) | |
| 44 | Hel308 Mev | Methanohalobium | 38% | 61% | QMAGRAGR | QMAGRAGRP |
| | | evestigatum Z- | | | (SEQ ID NO: | (SEQ ID NO: 12) |
| | | 7303 | | | 11) | |
| 45 | Hel308 Mma | Methanococcus | 36% | 32% | QCIGRAGR | QCIGRAGRP |
| | | maripaludis | | | (SEQ ID NO: | (SEQ ID NO: 31) |
| | | | | | 30) | |
| 46 | Hel308 Nma | Natrialba magadii | 37% | 43% | QMMGRAGR | QMMGRAGRP |
| | | | | | (SEQ ID NO: | (SEQ ID NO: 18) |
| | | | | | 17) | |
| 47 | Hel308 Mbo | Methanoregula | 38% | 45% | QMAGRAGR | QMAGRAGRP |
| | | boonei 6A8 | | | (SEQ ID NO: | (SEQ ID NO: 12) |
| | | | | | 11) | |
| 48 | Hel308 Fac | Ferroplasma | 34% | 32% | QMIGRAGR | QMIGRAGRP |
| | | acidarmanus | | | (SEQ ID NO: | (SEQ ID NO: 36) |
| | | | | | 35) | |
| 49 | Hel308 Mfe | Methanocaldococcus | 40% | 35% | QCIGRAGR | QCIGRAGRP |
| | | fervens AG86 | | | (SEQ ID NO: | (SEQ ID NO: 31) |
| | | | | | 30) | |
| 50 | Hel308 Mja | Methanocaldococcus | 24% | 22% | QCIGRAGR | QCIGRAGRP |
| | | jannaschii | | | (SEQ ID NO: | (SEQ ID NO: 31) |
| | | | | | 30) | |
| 51 | Hel308 Mm | Methanocaldococcus | 41% | 33% | QCIGRAGR | QCIGRAGRP |
| | | infernus | | | (SEQ ID NO: | (SEQ ID NO: 31) |
| | | | | | 30) | |
| 52 | Hel308 Mhu | Methanospirillum | 36% | 40% | QMAGRAGR | QMAGRAGRP |
| | | hungatei JF-1 | | | (SEQ ID NO: | (SEQ ID NO: 12) |
| | | | | | 11) | |
| 53 | Hel308 Afu | Archaeoglobus | 40% | 40% | QMAGRAGR | QMAGRAGRP |
| | | fulgidus DSM | | | (SEQ ID NO: | (SEQ ID NO: 12) |
| | | 4304 | | | 11) | |
| 54 | Hel308 Htu | Haloterrigena | 35% | 43% | QMAGRAGR | QMMGRAGRP |
| | | turkmenica | | | (SEQ ID NO: | (SEQ ID NO: 12) |
| | | | | | 11) | |
| 55 | Hel308 Hpa | Haladaptatus | 38% | 45% | QMFGRAGR | QMFGRAGRP |
| | DX253 | paucihalophilus | | | (SEQ ID NO: | (SEQ ID NO: 57) |
| | | | | | 56) | |
| 58 | ski2-like | Halobacterium sp. | 36.8% | 42.0% | QMFGRAGR | QMFGRAGRP |
| | helicase | NRC-1 | | | (SEQ ID NO: | (SEQ ID NO: 57) |
| | | | | | 56) | |

The Hel308 helicase more preferably comprises (a) the sequence of Hel308 Mbu (i.e. SEQ ID NO: 10) or a variant thereof, (b) the sequence of Hel308 Pfu (i.e. SEQ ID NO: 13) or a variant thereof, (c) the sequence of Hel308 Mok (i.e. SEQ ID NO: 29) or a variant thereof, (d) the sequence of Hel308 Mma (i.e. SEQ ID NO: 45) or a variant thereof, (e) the sequence of Hel308 Fac (i.e. SEQ ID NO: 48) or a variant thereof or (f) the sequence of Hel308 Mhu (i.e. SEQ ID NO: 52) or a variant thereof. The Hel308 helicase more preferably comprises the sequence shown in SEQ ID NO: 10 or a variant thereof.

The Hel308 helicase more preferably comprises (a) the sequence of Hel308 Tga (i.e. SEQ ID NO: 33) or a variant thereof, (b) the sequence of Hel308 Csy (i.e. SEQ ID NO: 22) or a variant thereof or (c) the sequence of Hel308 Mhu (i.e. SEQ ID NO: 52) or a variant thereof. The Hel308 helicase most preferably comprises the sequence shown in SEQ ID NO: 33 or a variant thereof.

A variant of a Hel308 helicase is an enzyme that has an amino acid sequence which varies from that of the wild-type helicase and which retains polynucleotide binding activity. In particular, a variant of any one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 is an enzyme that has an amino acid sequence which varies from that of any one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 and which retains polynucleotide binding activity. A variant of SEQ ID NO: 10 or 33 is an enzyme that has an amino acid sequence which varies from that of SEQ ID NO: 10 or 33 and which retains polynucleotide binding activity. The variant retains helicase activity. The variant must work in at least one of the two modes discussed below. Preferably, the variant works in both modes. The variant may include modifications that facilitate handling of the polynucleotide encoding the helicase and/or facilitate its activity at high salt concentrations and/or room temperature. Variants typically differ from the wild-type helicase in regions outside of the Hel308 motif or extended Hel308 motif discussed above. However, variants may include modifications within these motif(s).

Over the entire length of the amino acid sequence of any one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58, such as SEQ ID NO: 10 or 33, a variant will preferably be at least 30% homologous to that sequence based on amino acid identity. More preferably, the variant polypeptide may be at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of any one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58, such as SEQ ID NO: 10 or 33, over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NOs: 2 and 4.

A variant of any one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 preferably comprises the Hel308 motif or extended Hel308 motif of the relevant wild-type sequence. For instance, a variant of SEQ ID NO: 10 preferably comprises the Hel308 motif of SEQ ID NO: 10 (QMAGRAGR; SEQ ID NO: 11) or extended Hel308 motif of SEQ ID NO: 10 (QMAGRAGRP; SEQ ID NO: 12). The Hel308 motif and extended Hel308 motif of each of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 are shown in Table 5. However, a variant of any one SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 may comprise the Hel308 motif or extended Hel308 motif from a different wild-type sequence. For instance, a variant of SEQ ID NO: 10 may comprise the Hel308 motif of SEQ ID NO: 13 (QMLGRAGR; SEQ ID NO: 14) or extended Hel308 motif of SEQ ID NO: 13 (QMLGRAGRP; SEQ ID NO: 15). A variant of any one SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 may comprise any one of the preferred motifs shown in Table 5. Variants of any one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 may also include modifications within the Hel308 motif or extended Hel308 motif of the relevant wild-type sequence. Suitable modifications at X1 and X2 are discussed above when defining the two motifs.

A variant of SEQ ID NO: 10 may lack the first 19 amino acids of SEQ ID NO: 10 and/or lack the last 33 amino acids of SEQ ID NO: 10. A variant of SEQ ID NO: 10 preferably comprises a sequence which is at least 70%, at least 75%, at least 80%, at least 85%, at least 90% or more preferably at least 95%, at least 97%or at least 99% homologous based on amino acid identity with amino acids 20 to 211 or 20 to 727 of SEQ ID NO: 10.

The helicase may be covalently attached to the pore. The helicase is preferably not covalently attached to the pore. The application of a voltage to the pore and helicase typically results in the formation of a sensor that is capable of sequencing target polynucleotides. This is discussed in more detail below.

Any of the proteins described herein, i.e. the transmembrane protein pores or Hel308 helicases, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the pore or helicase. An example of this would be to react a gel-shift reagent to a cysteine engineered on the outside of the pore. This has been demonstrated as a method for separating hemolysin hetero-oligomers (Chem Biol. 1997 July; 4(7):497-505).

The pore and/or helicase may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Proteins may be made synthetically or by recombinant means. For example, the pore and/or helicase may be synthesized by in vitro translation and transcription (IVTT). The amino acid sequence of the pore and/or helicase may be modified to include non-naturally occurring amino acids or to increase the stability of the protein. When a protein is produced by synthetic means, such amino acids may be introduced during production. The pore and/or helicase may also be altered following either synthetic or recombinant production.

The pore and/or helicase may also be produced using D-amino acids. For instance, the pore or helicase may comprise a mixture of L-amino acids and D-amino acids. This is conventional in the art for producing such proteins or peptides.

The pore and/or helicase may also contain other non-specific modifications as long as they do not interfere with pore formation or helicase function. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

The pore and helicase can be produced using standard methods known in the art. Polynucleotide sequences encoding a pore or helicase may be derived and replicated using standard methods in the art. Polynucleotide sequences encoding a pore or helicase may be expressed in a bacterial host cell using standard techniques in the art. The pore and/or helicase may be produced in a cell by in situ expression of the polypeptide from a recombinant expression vector. The expression vector optionally carries an inducible promoter to control the expression of the polypeptide. These methods are described in described in Sambrook, J. and Russell, D. (2001). Molecular Cloning: A Laboratory Manual, 3rd Edition. Cold Spring Harbor Laboratory Press, Cold Spring Harbor, NY.

The pore and/or helicase may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

The method of the invention involves measuring one or more characteristics of the target polynucleotide. The method may involve measuring two, three, four or five or more characteristics of the target polynucleotide. The one or more characteristics are preferably selected from (i) the length of the target polynucleotide, (ii) the identity of the target polynucleotide, (iii) the sequence of the target polynucleotide, (iv) the secondary structure of the target polynucleotide and (v) whether or not the target polynucleotide is modified. Any combination of (i) to (v) may be measured in accordance with the invention.

For (i), the length of the polynucleotide may be measured using the number of interactions between the target polynucleotide and the pore.

For (ii), the identity of the polynucleotide may be measured in a number of ways. The identity of the polynucleotide may be measured in conjunction with measurement of the sequence of the target polynucleotide or without measurement of the sequence of the target polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively. the measurement of a particular electrical and/or optical signal in the method may identify the target polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106(19):7702-7, Lieberman K R et al, J Am Chem Soc. 2010; 132(50):17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the target polynucleotide is modified by methylation, by oxidation, by damage, with one or more proteins or with one or more labels. tags or spacers. Specific modifications will result in specific interactions with the pore which can be measured using the methods described below. For instance, methylcyotsine may be distinguished from cytosine on the basis of the current flowing through the pore during its interation with each nucleotide.

A variety of different types of measurements may be made. This includes without limitation: electrical measurements and optical measurements. Possible electrical measurements include: current measurements, impedance measurements, tunneling measurements (Ivanov A P et al., Nano Lett. 2011 Jan. 12; 11(1):279-85), and FET measurements (International Application WO 2005/124888). Optical measurements may be combined 10 with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al. J Am Chem Soc. 2010; 132(50):17961-72, and International Application WO-2000/28312. Alternatively, electrical measurements may be made using a multi-channel system, for example as described in International Application WO-2009/077734 and International Application WO-2011/067559.

In a preferred embodiment, the method comprises:
(a) contacting the target polynucleotide with a transmembrane pore and a Hel308 helicase such that the helicase controls the movement of the target polynucleotide through the pore and nucleotides in the target polynucleotide interact with the pore; and
(b) measuring the current passing through the pore during one or more interactions to measure one or more characteristics of the target polynucleotide and thereby characterising the target polynucleotide.

The methods may be carried out using any apparatus that is suitable for investigating a membrane/pore system in which a pore is inserted into a membrane. The method may be carried out using any apparatus that is suitable for transmembrane pore sensing. For example, the apparatus comprises a chamber comprising an aqueous solution and a barrier that separates the chamber into two sections. The barrier has an aperture in which the membrane containing the pore is formed.

The methods may be carried out using the apparatus described in International Application No. PCT/GB08/000562 (WO 2008/102120).

The methods may involve measuring the current passing through the pore during one or more interactions with the nucleotide(s). Therefore the apparatus may also comprise an electrical circuit capable of applying a potential and measuring an electrical signal across the membrane and pore. The methods may be carried out using a patch clamp or a voltage clamp. The methods preferably involve the use of a voltage clamp.

The methods of the invention may involve the measuring of a current passing through the pore during one or more interactions with the nucleotide. Suitable conditions for measuring ionic currents through transmembrane protein pores are known in the art and disclosed in the Example. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. As discussed above, Hel308 helicases surprisingly work under high salt concentrations. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18°° C. to 80°° C., 19°° C. to 70° C., or from 20° C. to 60°° C. The methods are typically carried out at room temperature. The methods are optionally carried out at a temperature that supports enzyme function, such as about 37° C.

The method is typically carried out in the presence of free nucleotides or free nucleotide analogues and an enzyme cofactor that facilitate the action of the helicase. The free nucleotides may be one or more of any of the individual nucleotides discussed above. The free nucleotides include, but are not limited to, adenosine monophosphate (AMP), adenosine diphosphate (ADP), adenosine triphosphate (ATP). guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and doxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the helicase to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

The target polynucleotide may be contacted with the Hel308 helicase and the pore in any order. In is preferred that, when the target polynucleotide is contacted with the Hel308 helicase and the pore, the target polynucleotide firstly forms a complex with the helicase. When the voltage is applied across the pore, the target polynucleotide/helicase complex then forms a complex with the pore and controls the movement of the polynucleotide through the pore.

As discussed above, Hel308 helicases may work in two modes with respect to the nanopore. First. the method is preferably carried out using the Hel308 helicase such that it moves the target sequence through the pore with the field resulting from the applied voltage. In this mode the 3′ end of the DNA is first captured in the nanopore, and the enzyme moves the DNA into the nanopore such that the target sequence is passed through the nanopore with the field until it finally translocates through to the trans side of the bilayer. Alternatively, the method is preferably carried out such that the enzyme moves the target sequence through the pore against the field resulting from the applied voltage. In this mode the 5′ end of the DNA is first captured in the nanopore, and the enzyme moves the DNA through the nanopore such that the target sequence is pulled out of the nanopore against the applied field until finally ejected back to the cis side of the bilayer.

The method of the invention most preferably involves a pore derived from MspA and a helicase comprising the sequence shown in SEQ ID NO: 8 or 10 or a variant thereof. Any of the embodiments discussed above with reference to MspA and SEQ ID NO: 8 and 10 may be used in combination.

### Other methods

The invention also provides a method of forming a sensor for characterising a target polynucleotide. The method comprises forming a complex between a pore and a Hel308 helicase. The complex may be formed by contacting the pore and the helicase in the presence of the target polynucleotide and then applying a potential across the pore. The applied potential may be a chemical potential or a voltage potential as described above. Alternatively, the complex may be formed by covalently attaching the pore to the helicase. Methods for covalent attachment are known in the art and disclosed, for example, in International Application Nos. PCT/GB09/001679 (published as WO 2010/004265) and PCT/GB10/000133 (published as WO 2010/086603) The complex is a sensor for characterising the target polynucleotide. The method preferably comprises forming a complex between a pore derived from Msp and a Hel308 helicase. Any of the embodiments discussed above with reference to the method of the invention equally apply to this method.

### Kits

The present invention also provides kits for characterising a target polynucleotide. The kits comprise (a) a pore and (b) a Hel308 helicase. Any of the embodiments discussed above with reference to the method of the invention equally apply to the kits.

The kit may further comprise the components of a membrane, such as the phospholipids needed to form a lipid bilayer.

The kits of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

### Apparatus

The invention also provides an apparatus for characterising a target polynucleotide. The apparatus comprises a plurality of pores and a plurality of a Hel308 helicase. The apparatus preferably further comprises instructions for carrying out the method of the invention. The apparatus may be any conventional apparatus for polynucleotide analysis, such as an array or a chip. Any of the embodiments discussed above with reference to the methods of the invention are equally applicable to the apparatus of the invention.

The apparatus is preferably set up to carry out the method of the invention.

The apparatus preferably comprises:
a sensor device that is capable of supporting the membrane and plurality of pores and being operable to perform polynucleotide characterising using the pores and helicases;
at least one reservoir for holding material for performing the characterising;
a fluidics system configured to controllably supply material from the at least one reservoir to the sensor device; and
a plurality of containers for receiving respective samples, the fluidics system being configured to supply the samples selectively from the containers to the sensor device. The apparatus may be any of those described in International Application No. No. PCT/GB08/004127 (published as WO 2009/077734), PCT/GB10/000789 (published as WO 2010/122293), International Application No. PCT/GB10/002206 (not yet published) or International Application No. PCT/US99/25679 (published as WO 00/28312).

### Internally Binding Molecular Motors

Molecular motors are commonly used as a means for controlling the translocation of a polymer, particularly a polynucleotide, through a nanopore. Surprisingly, the inventors have found that molecular motors which are capable of binding to a target polynucleotide at an internal nucleotide, i.e. a position other than a 5′ or 3′ terminal nucleotide, can provide increased read lengths of the polynucleotide as the molecular motor controls the translocation of the polynucleotide through a nanopore. The ability to translocate an entire polynucleotide through a nanopore under the control of a molecular motor allows characteristics of the polynucleotide, such as its sequence, to be estimated with improved accuracy and speed over known methods. This becomes more important as strand lengths increase and molecular motors are required with improved processivity. The molecular motor used in the invention is particularly effective in controlling the translocation of target polynucleotides of 500 nucleotides or more, for example 1000 nucleotides, 5000, 10000 or 20000 or more.

The invention thus provides a method of characterising a target polynucleotide, comprising:
(a) contacting the target polynucleotide with a transmembrane pore and a molecular motor which is capable of binding to the target polynucleotide at an internal nucleotide such that the molecular motor controls the movement of the target polynucleotide through the pore and nucleotides in the target polynucleotide interact with the pore; and
(b) measuring one or more characteristics of the target polynucleotide during one or more interactions and thereby characterising the target polynucleotide.

Any of the embodiments discussed above in relation to the Hel308 methods of the invention equally apply to this method of the invention.

A problem which occurs in sequencing polynucleotides, particularly those of 500 nucleotides or more, is that the molecular motor which is controlling translocation of the polynucleotide may disengage from the polynucleotide. This allows the polynucleotide to be pulled through the pore rapidly and in an uncontrolled manner in the direction of the applied field. Multiple instances of the molecular motor used in the invention bind to the polynucleotide at relatively short distances apart and thus the length of polynucleotide which can be pulled through the pore before a further molecular motor engages with the pore is relatively short.

An internal nucleotide is a nucleotide which is not a terminal nucleotide in the target polynucleotide. For example, it is not a 3′ terminal nucleotide or a 5′ terminal nucleotide. All nucleotides in a circular polynucleotide are internal nucleotides.

Generally, a molecular motor which is capable of binding at an internal nucleotide is also capable of binding at a terminal nucleotide, but the tendency for some molecular motors to bind at an internal nucleotide will be greater than others. For a molecular motor suitable for use in the invention, typically at least 10% of its binding to a polynucleotide will be at an internal nucleotide. Typically, at least 20%, at least 30%, at least 40% or at least 50% of its binding will be at an internal nucleotide. Binding at a terminal nucleotide may involve binding to both a terminal nucleotide and adjacent internal nucleotides at the same time. For the purposes of the invention, this is not binding to the target polynucleotide at an internal nucleotide. In other words, the molecular motor used in the invention is not only capable of binding to a terminal nucleotide in combination with one or more adjacent internal nucleotides. The molecular motor must be capable of binding to an internal nucleotide without concurrent binding to a terminal nucleotide.

A molecular motor which is capable of binding at an internal nucleotide may bind to more than one internal nucleotide. Typically, the molecular motor binds to at least 2 internal nucleotides, for example at least 3, at least 4, at least 5, at least 10 or at least 15 internal nucleotides. Typically the molecular motor binds to at least 2 adjacent internal nucleotides, for example at least 3, at least 4, at least 5, at least 10 or at least 15 adjacent internal nucleotides. The at least 2 internal nucleotides may be adjacent or non-adjacent.

The ability of a molecular motor to bind to a polynucleotide at an internal nucleotide may be determined by carrying out a comparative assay. The ability of a motor to bind to a control polynucleotide A is compared to the ability to bind to the same polynucleotide but with a blocking group attached at the terminal nucleotide (polynucleotide B). The blocking group prevents any binding at the terminal nucleotide of strand B, and thus allows only internal binding of a molecular motor. An example of this type of assay is disclosed in Example 4.

Suitable molecular motors are well known in the art and typically include, but are not limited to, single and double strand translocases, such as polymerases, helicases, topoisomerases. ligases and nucleases, such as exonucleases. Preferably the molecular motor is a helicase, for example a Hel308 helicase. Examples of Hel308 helicases which are capable of binding at an internal nucleotide include, but are not limited to, Hel308 Tga, Hel308 Mhu and Hel308 Csy. Hence, the molecular motor preferably comprises (a) the sequence of Hel308 Tga (i.e. SEQ ID NO: 33) or a variant thereof or (b) the sequence of Hel308 Cy (i.e. SEQ ID NO: 22) or a variant thereof or (c) the sequence of Hel308 Mhu (i.e. SEQ ID NO: 52) or a variant thereof. The variant typically has at least 40% homology to SEQ ID NO: 33, 22 or 52 based on amino acid identity over the entire sequence and retains helicase activity. Further possible variants are discussed above.

The molecular motor used in the invention may be made by any of the methods discussed above and may be modified or labelled as discussed above. The molecular motor may be used in the methods described herein or as part of the apparatus described herein. The invention further provides a method of forming a sensor for characterising a target polynucleotide, comprising forming a complex between a pore and a molecular motor which is capable of binding to the target polynucleotide at an internal nucleotide and thereby forming a sensor for characterising the target polynucleotide. The invention also provides use of a molecular motor which is capable of binding to the target polynucleotide at an internal nucleotide to control the movement of a target polynucleotide through a pore. The invention also provides a kit for characterising a target polynucleotide comprising (a) a pore and (b) a molecular motor which is capable of binding to the target polynucleotide at an internal nucleotide. The invention also provides an analysis apparatus for characterising target polynucleotides in a sample, comprising a plurality of pores and a plurality of a molecular motor which is capable of binding to the target polynucleotide at an internal nucleotide.

The following Examples illustrate the invention.

### Example 1

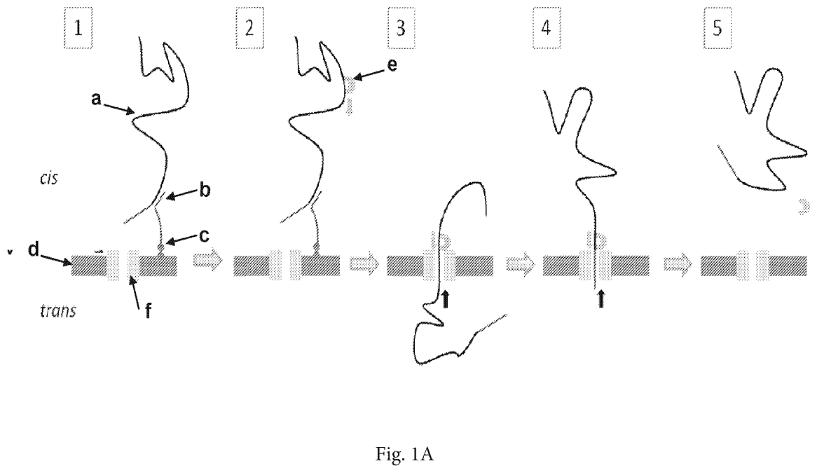

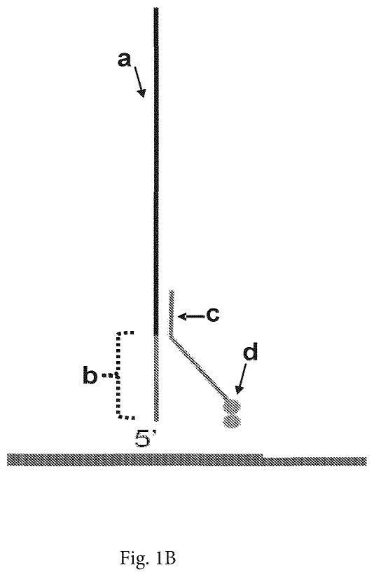

This Example illustrates the use of a Hel308 helicase (Hel308 MBu) to control the movement of intact DNA strands through a nanopore. The general method and substrate employed throughout this example is shown in FIG. 1 and described in the figure caption.

### Materials and Methods

Primers were designed to amplify a ˜400 bp fragment of PhiX174. Each of the 5′-ends of these primers included a 50 nucleotide non-complimentary region, either a homopolymeric stretch or repeating units of 10 nucleotide homopolymeric sections. These serve as identifiers for controlled translocation of the strand through a nanopore, as well as determining the directionality of translocation. In addition, the 5′-end of the forward primer was “capped” to include four 2′-O-Methyl-Uracil (mU) nucleotides and the 5′-end of the reverse primer was chemically phosphorylated. These primer modifications then allow for the controlled digestion of predominantly only the antisense strand, using lambda exonuclease. The mU capping protects the sense strand from nuclease digestion whilst the PO4 at the 5′ of the antisense strand promotes it. Therefore after incubation with lambda exonuclease only the sense strand of the duplex remains intact, now as single stranded DNA (ssDNA). The generated ssDNA was then PAGE purified as previously described.

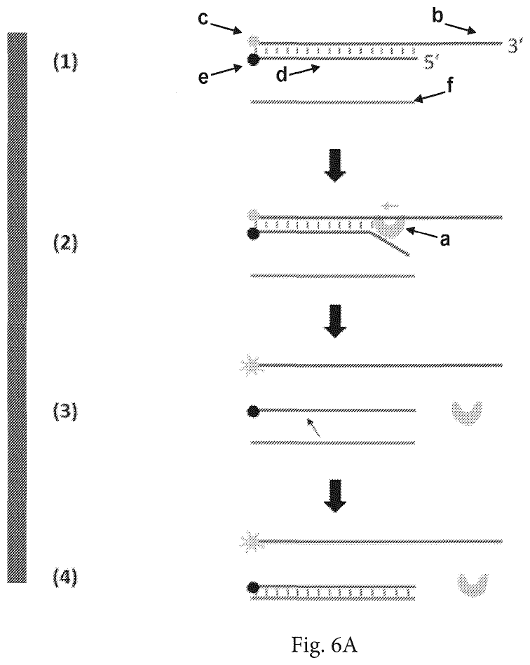

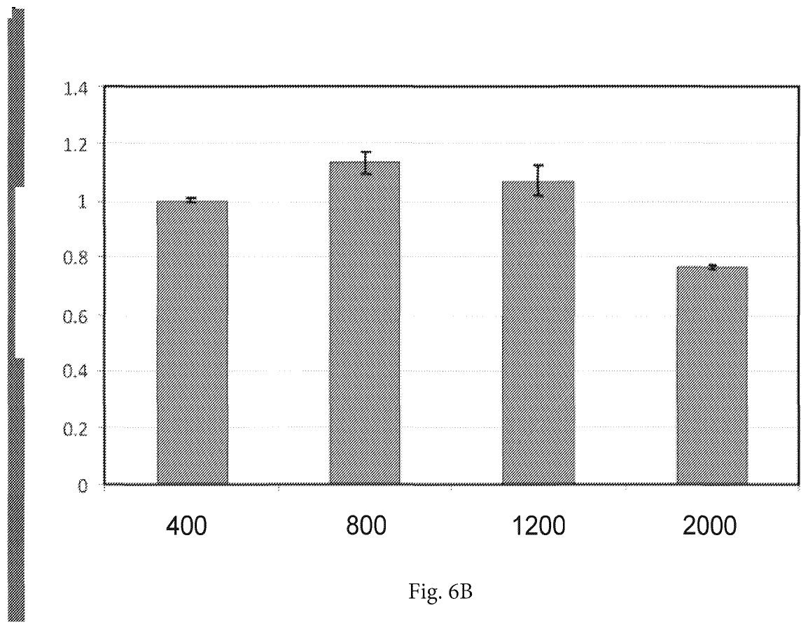

The DNA substrate design used in all the experiments described here is shown in FIG. 6. The DNA substrate consists of a 400 base section of ssDNA from PhiX, with a 50T 5′-leader to aid capture by the nanopore (SEQ ID NO: 59). Annealed to this strand just after the 50T leader is a primer (SEQ ID NO: 60) containing a 3′ cholesterol tag to enrich the DNA on the surface of the bilayer, and thus improve capture efficiency.
Buffered solution: 400 mM-2 M KCl, 10 mM Hepes pH 8.0, 1 mM ATP, 1 mM MgCl₂, 1 mM DTT
Nanopore: E. coli MS(B2)8 MspA ONLP3271 MS-(L88N/D90N/D91N/D93N/D118R/D134R/E139K)8
Enzyme: Hel308 Mbu (ONLP3302, ˜7.7 μM) 12.5 μl->100 nM final.

Electrical measurements were acquired from single MspA nanopores inserted in 1,2-diphytanoyl-glycero-3-phosphocholine lipid (Avanti Polar Lipids) bilayers. Bilayers were formed across ˜100 μm diameter apertures in 20 μm thick PTFE films (in custom Delrin chambers) via the Montal-Mueller technique, separating two 1 mL buffered solutions. All experiments were carried out in the stated buffered solution. Single-channel currents were measured on Axopatch 200B amplifiers (Molecular Devices) equipped with 1440A digitizers. Ag/AgCl electrodes were connected to the buffered solutions so that the cis compartment (to which both nanopore and enzyme/DNA are added) is connected to the ground of the Axopatch headstage, and the trans compartment is connected to the active electrode of the headstage. After achieving a single pore in the bilayer, DNA polynucleotide and helicase were added to 100 μL of buffer and pre-incubated for 5 mins (DNA=1.5 nM, Enzyme=1 μM). This pre-incubation mix was added to 900 μL of buffer in the cis compartment of the electrophysiology chamber to initiate capture of the helicase-DNA complexes in the MspA nanopore (to give final concentrations of DNA=0.15 nM, Enzyme=0.1 μM). Helicase ATPase activity was initiated as required by the addition of divalent metal (1 mM MgCl₂) and NTP (1 mM ATP) to the cis compartment. Experiments were carried out at a constant potential of +180 mV.

### Results and Discussion

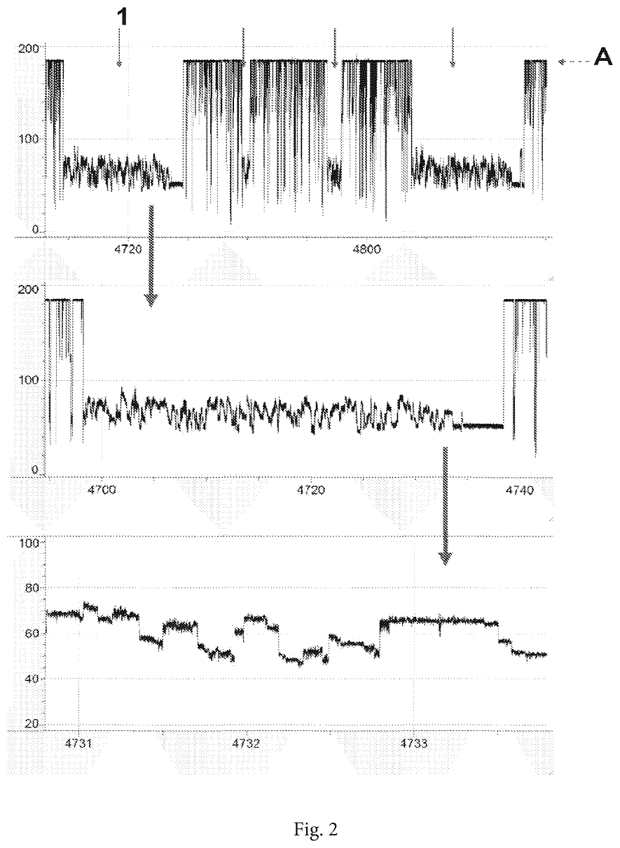

The addition of Helicase-DNA substrate to MspA nanopores as shown in FIG. 1 produces characteristic current blocks as shown in FIG. 2. DNA without helicase bound interacts transiently with the nanopore producing short-lived blocks in current (<<1 second). DNA with helicase bound and active (ie. moving along the DNA strand under ATPase action) produces long characteristic blocks levels with stepwise changes in current as shown in FIG. 2. Different DNA motifs in the nanopore give rise to unique current block levels.

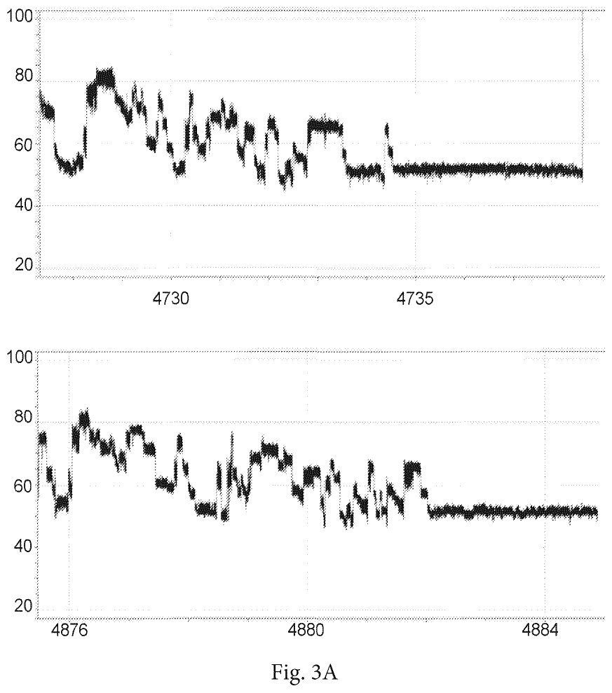

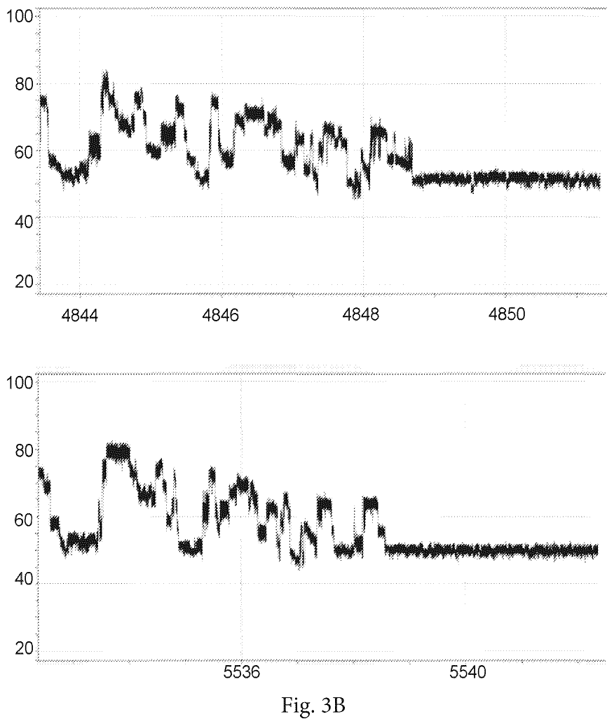

For a given substrate, we observe a characteristic pattern of current transitions that reflects the DNA sequence (examples in FIG. 3).

In the implementation shown in FIG. 1, the DNA strand is sequenced from a random starting point as the DNA is captured with a helicase at a random position along the strand. However, as long as the enzyme does not dissociate, the strands will all end in the same way at the 50T leader (FIG. 1). As FIG. 2 shows, we observe the same characteristic ending to most strands, with the current transitions ending in a long dwell time polyT level (FIG. 3).

### Salt Tolerance

Nanopore strand sequencing experiments of this type require ionic salts. The ionic salts are necessary to create a conductive solution for applying a voltage offset to capture and translocate DNA, and to measure the resulting sequence dependent current changes as the DNA passes through the nanopore. Since the measurement signal is dependent in the concentration of the ions, it is advantageous to use high concentration ionic salts to increase the magnitude of the acquired signal. For nanopore sequencing salt concentrations in excess of 100 mM KCl are ideal, and salt concentrations of 1 M KCl and above are preferred.

However, many enzymes (including some helicases and DNA motor proteins) do not tolerate high salt conditions. Under high salt conditions the enzymes either unfold or lose structural integrity, or fail to function properly. The current literature for known and studied helicases shows that almost all helicases fail to function above salt concentrations of approximately 100 mM KCl/NaCl, and there are no reported helicases that show correct activity in conditions of 400 mM KCl and above. While potentially halophilic variants of similar enzymes from halotolerant species exist, they are extremely difficult to express and purify in standard expression systems (e.g. E. coli).

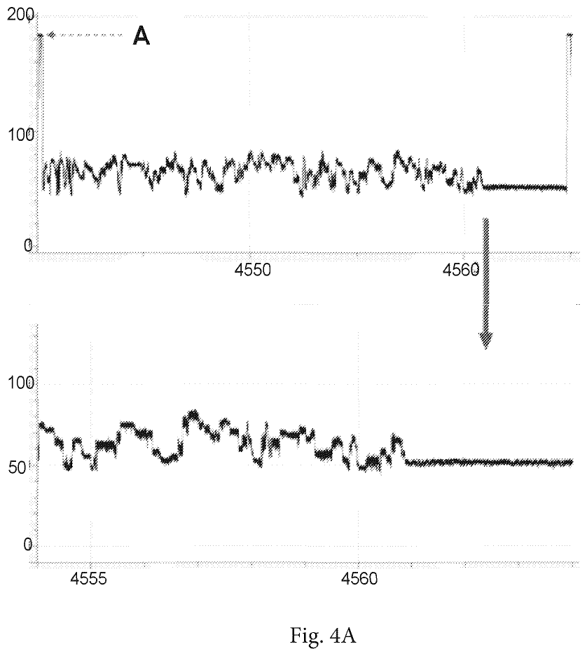

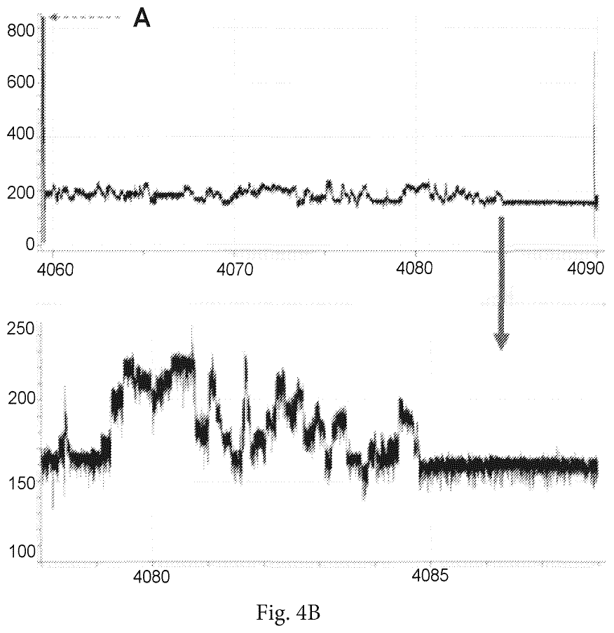

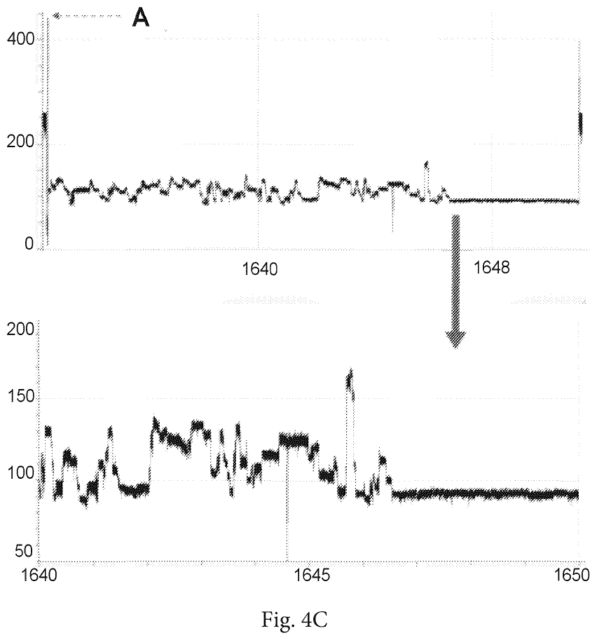

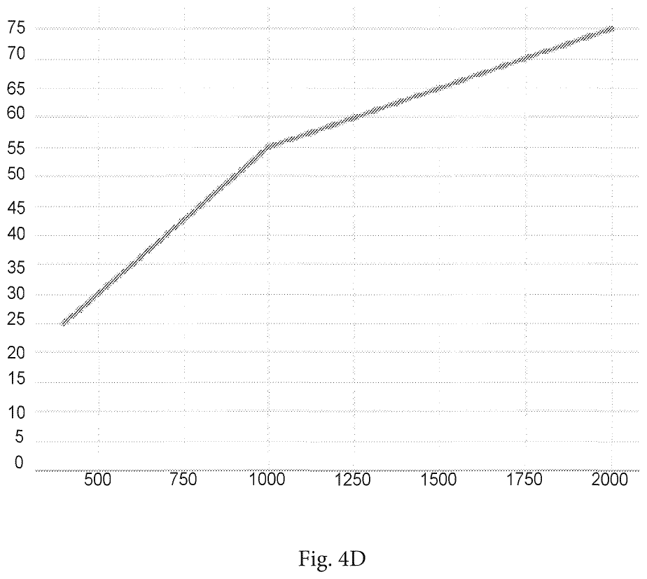

We surprisingly show in this Example that Hel308 from Mbu displays salt tolerance up to very high levels of KCl. We find that the enzyme retains functionality in salt concentrations of 400 mM KCl through to 2 M KCl, either in fluorescence experiments or in nanopore experiments (FIG. 4). FIG. 4 shows the Hel308 Mbu DNA events at 400 mM KCl, 1 M KCl, and 2 M KCl salt conditions carried out using the same system described in FIG. 1. We observe similar movement across the range of salt concentrations. As the salt concentration is increased we observe an increase in the current through the nanopore (I-open) at a fixed voltage. This reflects the increase in the conductivity of the solution and the increased number of ions flowing through the nanopore under the applied field. In addition we also observe an increase in the minimum to maximum range of discrimination in the current levels of the DNA events (see FIG. 4 enlargements and bottom right plot). We observe a ˜200% increase in DNA discrimination range as the salt concentration is increased from 400 mM KCl to 2M KCl (Table 6 below).

TABLE 6
Effect of increasing salt concentration
on pore current and DNA range

| Salt (KCl) | Open-pore current | DNA |
|---|---|---|
| (M) | (pA) | range (pA) |
| 0.4 | 180 | 25 |
| 1.0 | 440 | 55 |
| 2.0 | 840 | 75 |

### Forward and Reverse Modes of Operation

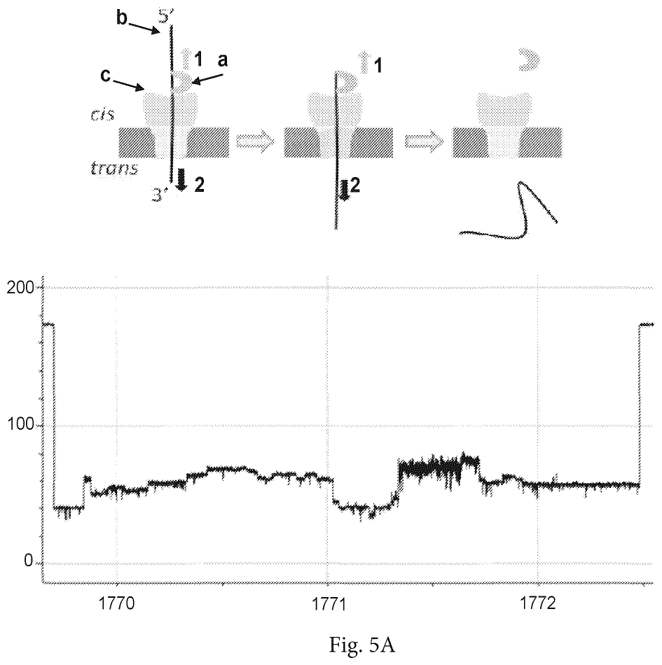

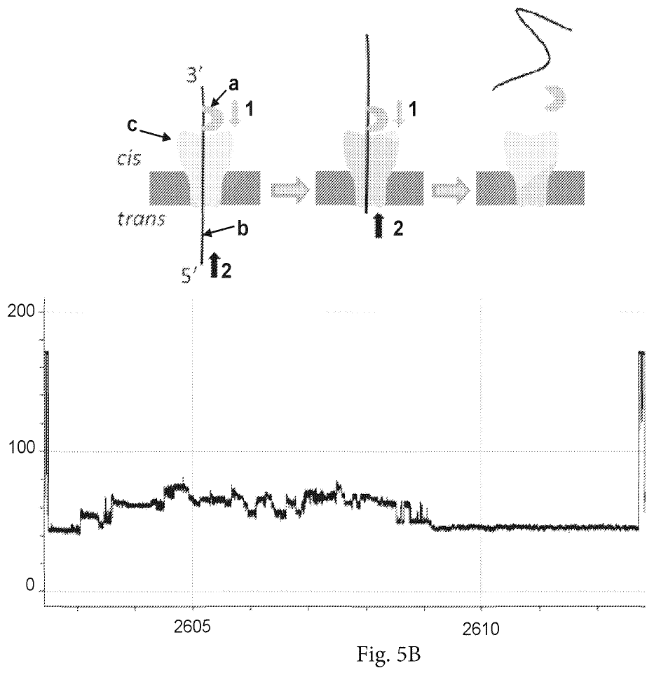

Most helicases move along single-stranded polynucleotide substrates in uni-directional manner, moving a specific number of bases for each NTPase turned over. Although FIG. 1 illustrates the use of this movement to pull threaded DNA out of the nanopore, helicase movement could be exploited in other manners to feed DNA through the nanopore in a controlled fashion. FIG. 5 illustrates the basic ‘forward’ and ‘reverse’ modes of operation. In the forward mode, the DNA is fed into the pore by the helicase in the same direction as the DNA would move under the force of the applied field. For Hel308 Mbu, which is a 3′-5′ helicase, this requires capturing the 3′ end of the DNA in the nanopore until a helicase contacts the top of the nanopore, and the DNA is then fed into the nanopore under the control of the helicase with the field from the applied potential, finally exiting on the trans side of the bilayer. The reverse mode requires capturing the 5′ end of the DNA, after which the helicase proceeds to pull the threaded DNA back out of the nanopore against the field from the applied potential, finally ejecting it on this cis side of the bilayer. FIG. 5 shows these two modes of operation using Hel308 Mbu, and typical example DNA events.

### Example 2

This Example illustrates the salt tolerance of a Hel308 helicase (Hel308 MBu) using a fluorescence assay for testing enzyme activity.

A custom fluorescent substrate was used to assay the ability of the helicase to displace hybridized dsDNA (FIG. 6A). As shown in 1) of FIG. 6A, the fluorescent substrate strand (100 nM final) has a 3′ ssDNA overhang, and a 40 base section of hybridized dsDNA. The major upper strand has a carboxyfluorescein base at the 5′ end, and the hybrised complement has a black-hole quencher (BHQ-1) base at the 3′ end. When hybrised the fluorescence from the fluorescein is quenched by the local BHQ-1. and the substrate is essentially non-fluorescent. 1 μM of a capture strand that is complementary to the shorter strand of the fluorescent substrate is included in the assay. As shown in 2), in the presence of ATP (1 mM) and MgCl₂ (5 mM), helicase (100 nM) added to the substrate binds to the 3′ tail of the fluorescent substrate, moves along the major strand, and displaces the complementary strand as shown. As shown in 3), once the complementary strand with BHQ-1 is fully displaced the fluorescein on the major strand fluoresces. As shown in 4), an excess of capture strand preferentially anneals to the complementary DNA to prevent re-annealing of initial substrate and loss of fluorescence.

Substrate DNA: 5′FAM-SEQ ID NO: 61 and SEQ ID NO: 62-BHQ1-3 . FAM=carboxyfluorescein and BHQ1=Black Hole Quencher-1

Capture DNA: SEQ ID NO: 62.

The graph in FIG. 6 shows the initial rate of activity in buffer solutions (10 mM Hepes pH 8.0, 1 mM ATP, 5 mM MgCl₂, 100 nM fluorescent substrate DNA, 1 μM capture DNA) containing different concentrations of KCl from 400 mM to 2 M. The helicase works at 2 M.

### Example 3

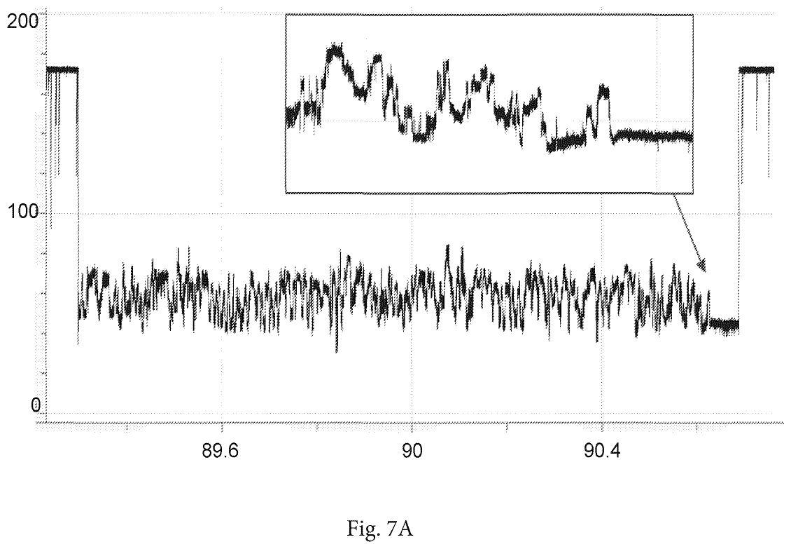

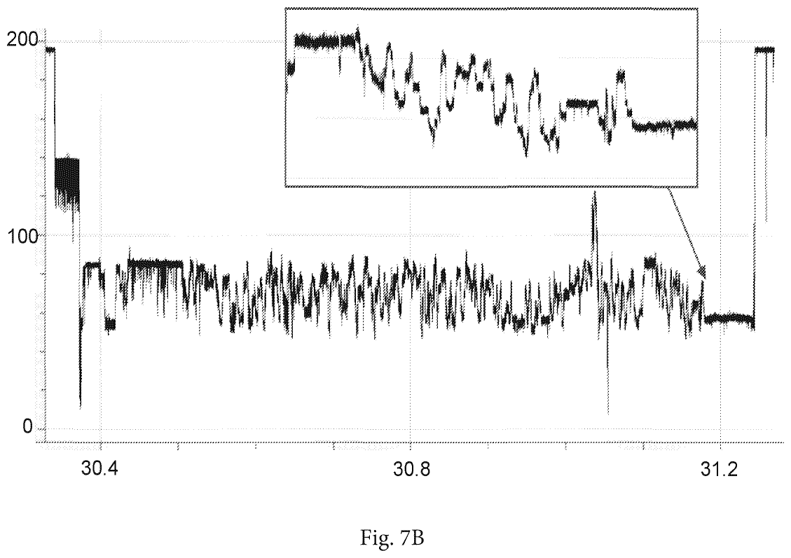

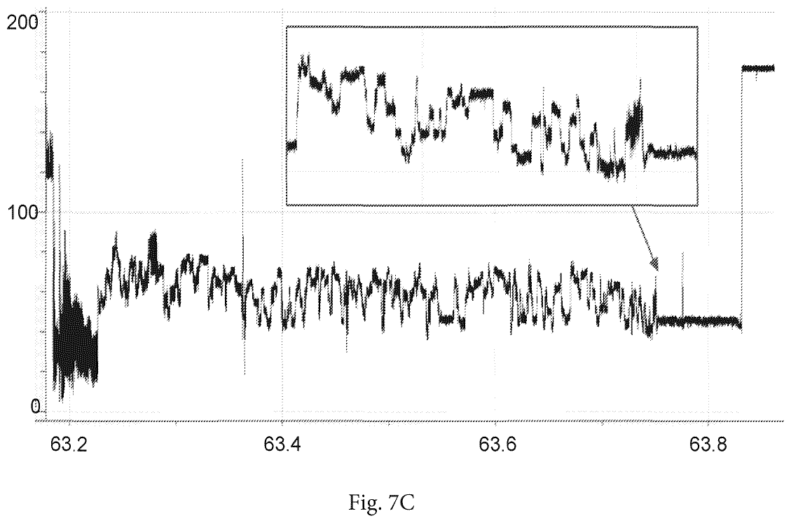

In this Example, three different Hel308 helicases were used, namely Hel308 Mhu (SEQ ID NO: 52), Hel308 Mok (SEQ ID NO: 29) and Hel308 Mma (SEQ ID NO: 45). All experiments were carried out as previously described in Example 1 under the same experimental conditions (pore=MspA B2, DNA=400 mer SEQ ID NO: 59 and 60, buffer=400 mM KCl, 10 mM Hepes pH 8.0. 1 mM dtt, 1 mM ATP, 0.1 mM MgCl₂). The results are shown in FIG. 7.

### Example 4

This Example measures the internal binding capabilities of a number of Hel308 helicases using a fluorescence assay.

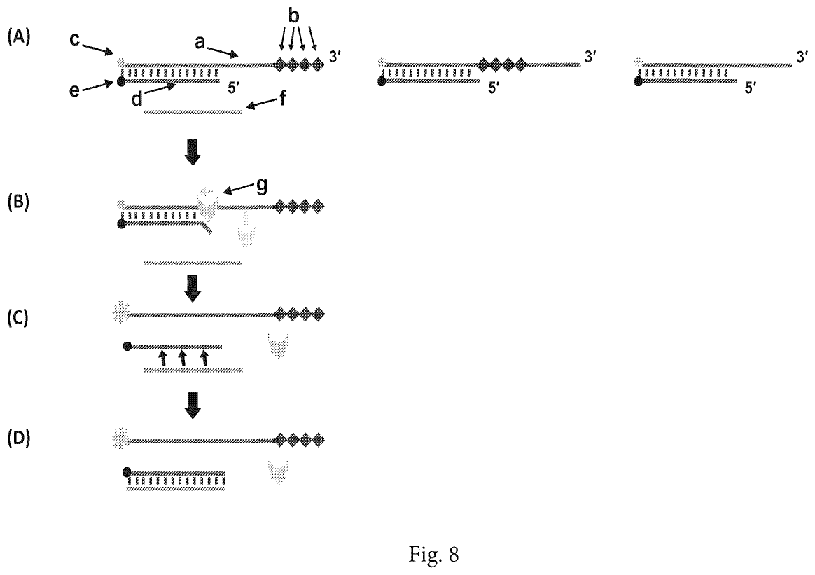

Custom fluorescent substrates were used to assay the ability of the helicases to initiate on DNA lacking native 3′ ends, allowing them to subsequently displace hybridized dsDNA (FIG. 8). As shown in section A of FIG. 8, the fluorescent substrate strand (50 nM final) has a 3′ ssDNA overhang, and a 40 base section of hybridized dsDNA. The major upper strands are modified with four consecutive “spacer 9” groups, either at the 3′ end, or internally, at the junction between the overhang and the dsDNA (as a negative control). Furthermore, the major upper strand has a carboxyfluorescein base at the 5′ end, and the hybridized complement has a black-hole quencher (BHQ-1) base at the 3′ end. When hybridized, the fluorescence from the fluorescein is quenched by the local BHQ-1, and the substrate is essentially non-fluorescent. A capture strand (1 μM), that is complementary to the shorter strand of the fluorescent substrate, is included in the assay. In the presence of ATP (1 mM) and MgCl₂ (1 mM), a Hel308 helicase homologue (20 nM), added to the substrate containing 3′-terminal “spacer 9” groups, can bind to the ssDNA overhang of the fluorescent substrate, move along the major strand, and displace the complementary strand as shown in section B. Once the complementary strand with BHQ-1 is fully displaced (section C) the fluorescein on the major strand fluoresces. An excess of capture strand preferentially anneals to the complementary DNA to prevent re-annealing of initial substrate and loss of fluorescence (section D).

Substrate DNA: SEQ ID NO: 63 with a 5′ FAM; SEQ ID NO: 63 with a 5′ FAM and 3′ spacer ((spacer 9)₄); SEQ ID NOs: 64 (with a 5′ FAM) and 65 separated by a spacer ((spacer 9)₄); and SEQ ID NO: 62 with a 3′ BHQ1.

Capture DNA: SEQ ID NO: 66.

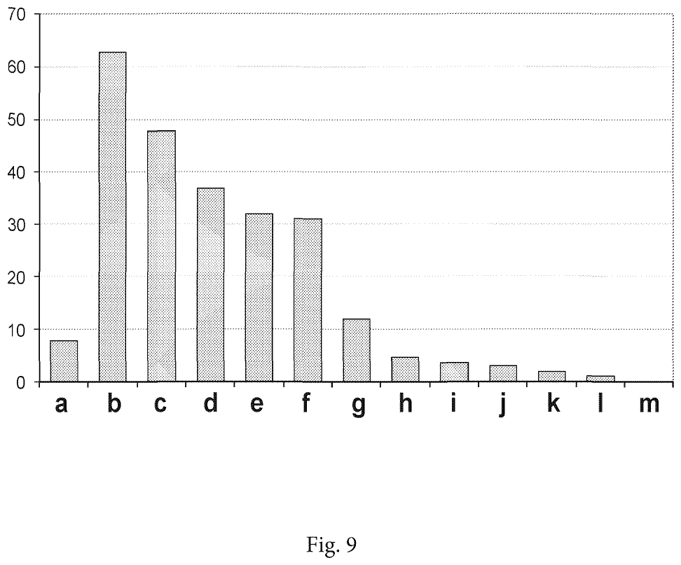

A number of different Hel308 helicase homologues were investigated for their mid-binding abilities, these included Hel308 Mbu, Hel308 Csy, Hel308 Tga, Hel308 Mma, Hel308 Mhu, Hel308 Min, Hel308 Mig, Hel308 Mmaz, Hel308 Mac, Hel308 Mok, Hel308 Mth, Hel308 Mba and Hel308 Mzh. The graph in FIG. 9 shows the relative rates of Hel308-mediated dsDNA turnover, comparing 3′-unmodified DNA and 3′-“spacer 9” DNA in 400 mM NaCl, 10 mM Hepes, pH 8.0, 1 mM ATP, 1 mM MgCl₂, 50 nM fluorescent substrate DNA, 1 μM capture DNA. Several Hel308 homologues were observed to have greater than 20% relative rates of Hel308-mediated dsDNA turnover including, Hel308 Csy, Hel308 Tga, Hel308 Mma, Hel308 Mhu and Hel308 Min.

### Example 5

This Example compares the use of two Hel308 helicases, Hel308 MBu and Hel 308 Tga, and their ability to control the movement of intact long DNA strands (900 mer) through a nanopore. The general method and substrate employed throughout this Example are shown in FIG. 10 and described in the description of the Figure above.

Materials and Methods

The DNA was formed by ligating a 50-polyT 5′ leader to a ˜900 base fragment of PhiX dsDNA. The leader also contains a complementary section to which SEQ ID NO: 69 with a Chol-tag was hybridized to allow the DNA to be tethered to the bilayer. Finally the 3′ end of the PhiX dsDNA was digested with AatII digestion enzyme to yield a 4 nt 3′-overhang of ACGT.

Sequences used: SEQ ID NO: 67-900 mer sense strand including 5′ leader and tether; SEQ ID NO: 68—anti-sense minus 4 base-pair leader 5′; and SEQ ID NO: 69 with several spacers and a Chol-tag at the 3′ end.
Buffered solution: 400 mM-2 NaCl, 10 mM potassium ferrocyanide, 10 mM potassium ferricyanide, 100 mM Hepes, pH 8.0, 1 mM ATP, 1 mM MgCl2,
Nanopore: MS-(B1-G75S-G77S-L88N-Q126R)8 (ONT Ref B2C)
Enzyme: Hel308 Mbu 1000 nM or Hel308 Tga 400 nM final.

Electrical experiments were set up as described in Example 1 in order to achieve a single pore inserted into a lipid bilayer. After achieving a single pore in the bilayer, ATP (1 mM) and MgCl₂ (1 mM) were added to the chamber. A control recording at +140 mV was run for 2 minutes. DNA polynucleotide SEQ ID NOs: 67, 68 and 69 (DNA=0.15 nM) were then added and DNA events observed. Finally, Hel308 helicase (Mbu 1000 nM or Tga, 400 nM) was added to the cis compartment of the electrophysiology chamber to initiate capture of the helicase-DNA complexes in the MspA nanopore. Experiments were carried out at a constant potential of +140 mV.

### Results and Discussion

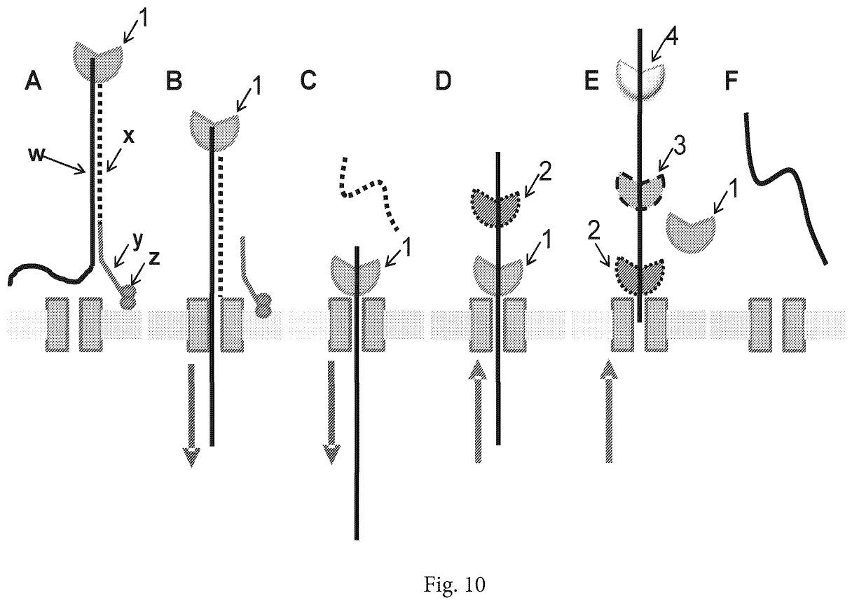

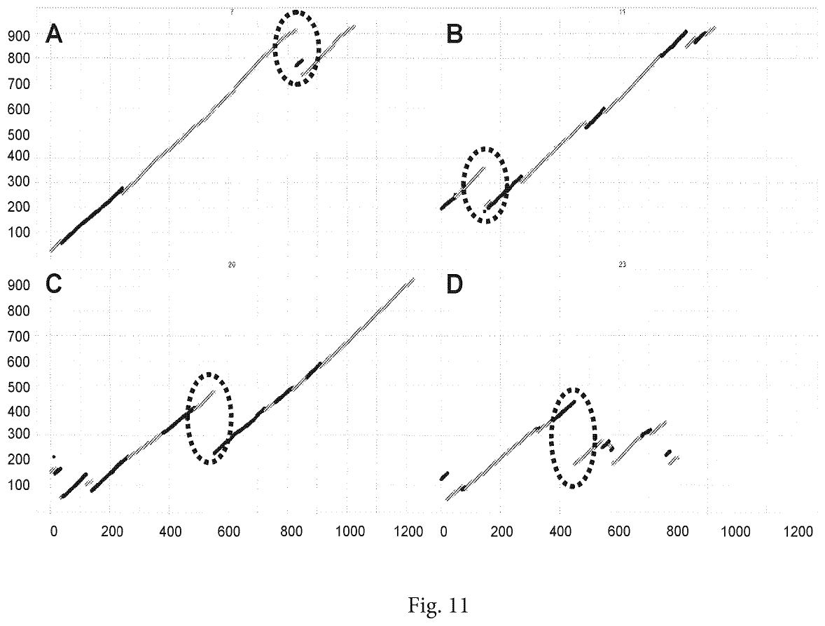

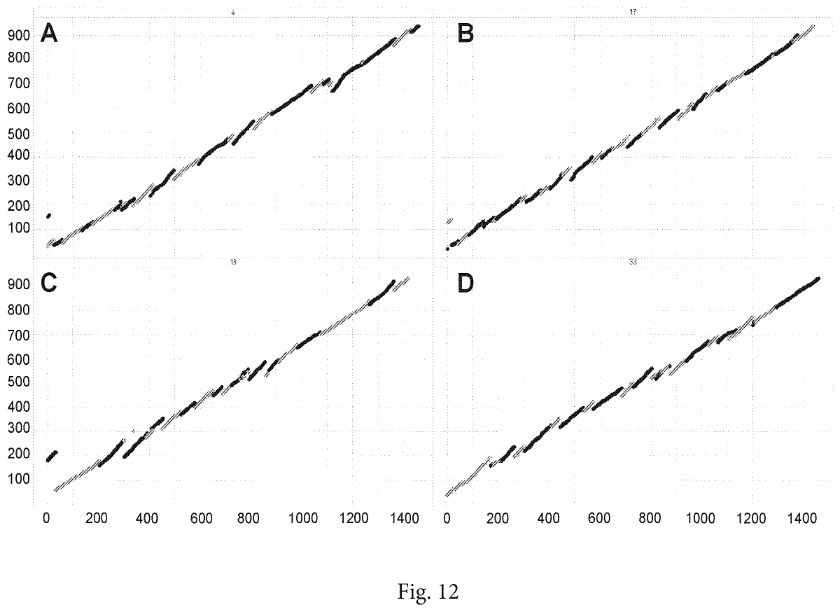

The addition of Helicase-DNA substrate to MspA nanopores as shown in FIG. 10 produces characteristic current blocks as the helicase controls the translocation of the DNA through the pore. FIG. 11 shows example event traces which indicate how the position of the 900 mer varied as the Hel308 helicase homologue Mbu controlled the translocation of the DNA strand through the MspA pore. This helicase was found to mediate control of DNA translocation, however, when the helicase detached from the DNA, the strand was observed to move back through the pore, owing to the force exerted by the externally applied potential. In the case of the Hel308 helicase homologue Mbu, the 900 mer strand slipped back a large number of positions (approximately 100-200 bases) each time a helicase disengaged. These rapid changes in position are indicated in FIG. 11 by dotted circles. For this experiment, where Hel308 helicase homologue Mbu was used as the molecular motor, 32% of all of the events detected were found to have read the entire length of the 900 mer strand sequence. FIG. 12 shows similar example event traces indicating how the position of the 900 mer varied as the Hel308 helicase homologue Tga controlled the translocation of the DNA strand through the MspA pore. This enzyme exhibited an greater tendency to bind internally, than the Mbu homologue, because when the Tga helicase disengages (indicated by a change in colour black to grey in FIG. 12), the DNA strand moves back through the pore by a relatively small distance (<50 bases). For this experiment, where Hel308 helicase homologue Tga was used as the molecular motor, 74% of all of the events detected were found to have read the entire length of the 900 mer strand sequence. This means that the Tga helicase homologue can provide increased read lengths of single-stranded DNA in comparison to the Mbu helicase homologue owing to its increased tendency to bind internally.

### Example 6

This Example illustrates that by employing the Hel308 helicase homologue Tga it is possible to control the translocation of a 5 kb strand of DNA.

A similar experimental procedure was followed to that described in Example 5. It was observed that by employing the Hel308 Tga it was possible to detect the controlled translocation of an entire 5 kb strand of DNA through MS-(B1-G75S-G77S-L88N-Q126R)8. In an identical experiment using Hel308 Mbu, it was not possible to detect translocation of an entire 5 kB strand.

### Example 7

This example compares the enzyme processivity of Hel308 Mbu helicase (SEQ ID NO: 10) with Hel308 Mok (SEQ ID NO: 29) using a fluorescence based assay.

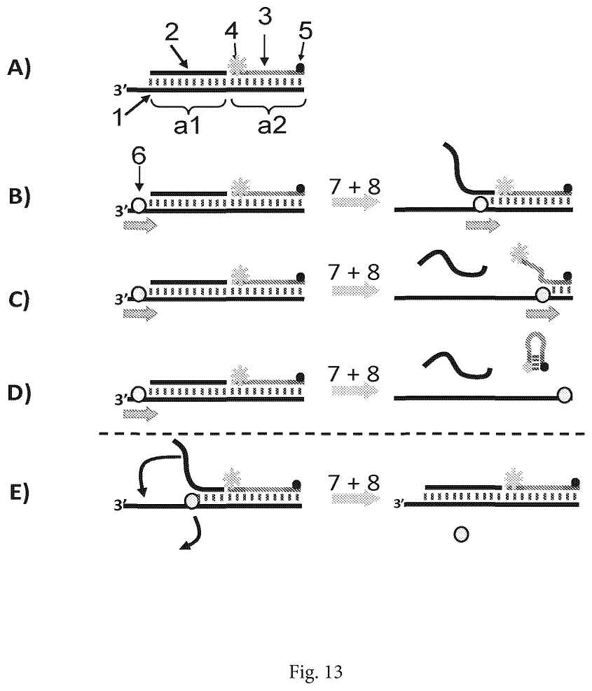

A custom fluorescent substrate was used to assay the ability of the helicase to displace hybridized dsDNA (FIG. 3). The fluorescent substrate (50 nM final) has a 3′ ssDNA overhang, and 80 and 33 base-pair sections of hybridized dsDNA (FIG. 13 section A, SEQ ID NO: 70). The major lower “template” strand is hybridized to an 80 nt “blocker” strand (SEQ ID NO: 71), adjacent to its 3′ overhang, and a 33 nt fluorescent probe, labelled at its 5′ and 3′ ends with carboxyfluorescein (FAM) and black-hole quencher (BHQ-1) bases, respectively (SEQ ID NO: 72). When hybridized, the FAM is distant from the BHQ-1 and the substrate is essentially fluorescent. In the presence of ATP (1 mM) and MgCl₂ (10 mM), the helicase (20 nM) binds to the substrate's 3′ overhang (SEQ ID NO: 70), moves along the lower strand, and begins to displace the 80 nt blocker strand (SEQ ID NO: 71), as shown in FIG. 13 section B. If processive, the helicase displaces the fluorescent probe (SEQ ID NO: 72, labeled with a carboxyfluorescein (FAM) at its 5′ end a black-hole quencher (BHQ-1) at its 3′ end) too (FIG. 13 section C). The fluorescent probe is designed in such a way that its 5′ and 3′ ends are self-complementary and thus form a kinetically-stable hairpin once displaced, preventing the probe from re-annealing to the template strand (FIG. 13 section D). Upon formation of the hairpin product, the FAM is brought into the vicinity of the BHQ-1 and its fluorescence is quenched. A processive enzyme, capable of displacing the 80 mer “blocker” (SEQ ID NO: 71) and fluorescent (SEQ ID NO: 72, labeled with a carboxyfluorescein (FAM) at its 5′ end a black-hole quencher (BHQ-1) at its 3′ end) strands will therefore lead to a decrease in fluorescence over time. However, if the enzyme has a processivity of less than 80 nt it would be unable to displace the fluorescent strand (SEQ ID NO: 72, labeled with a carboxyfluorescein (FAM) at its 5′ end a black-hole quencher (BHQ-1) at its 3′ end) and, therefore, the “blocker” strand (SEQ ID NO: 71) would reanneal to the major bottom strand (FIG. 13 section E, SEQ ID NO: 70).

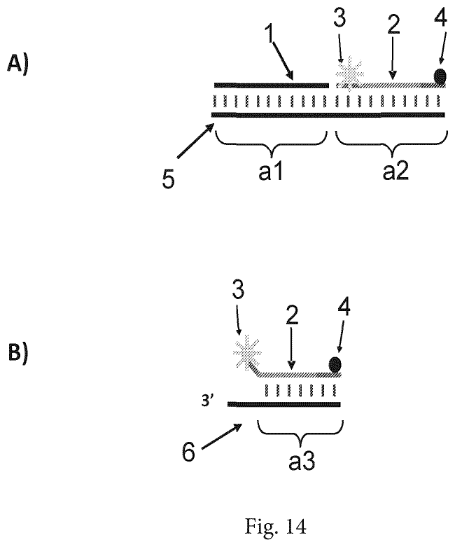

Additional custom fluorescent substrates were also used for control purposes. The substrate used as a negative control was identical to that of the one described in FIG. 13 but lacking the 3′overhang (FIG. 14 section A, (SEQ ID NOs: 71, 72 (labeled with a carboxyfluorescein (FAM) at its 5′ end a black-hole quencher (BHQ-1) at its 3′ end) and 73)). A similar substrate to that described in FIG. 13 but lacking the 80 base pair section, used as a positive control for active, but not necessarily processive, helicases (FIG. 14 section B, (SEQ ID NOs: 72 (labeled with a carboxyfluorescein (FAM) at its 5′ end a black-hole quencher (BHQ-1) at its 3′ end) and 74)).

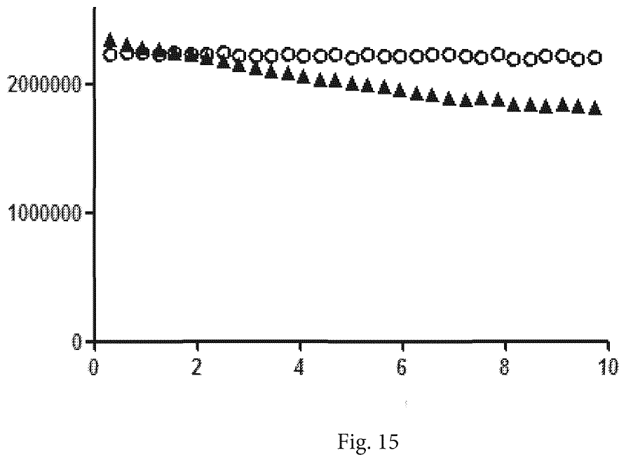

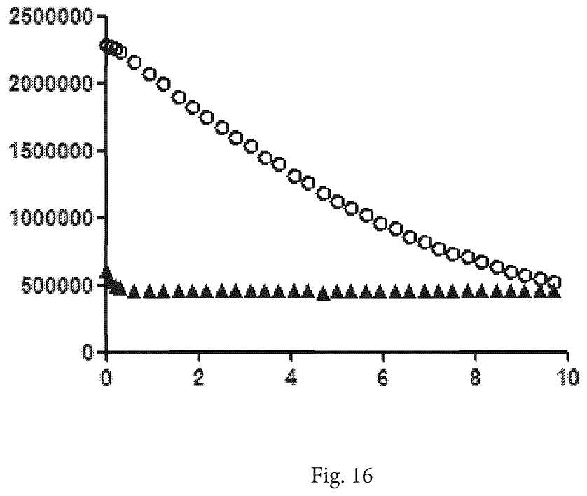

FIG. 15 shows a graph of the time-dependent fluorescence changes upon testing Hel308 Mbu helicase (SEQ ID NO: 10) and Hel 308 Mok helicase (SEQ ID NO: 29) against the processivity substrate shown in FIG. 13 in buffered solution (400 mM NaCl, 10 mM Hepes pH 8.0, 1 mM ATP, 10 mM MgCl₂, 50 nM fluorescent substrate DNA (SEQ ID NOs: 70, 71 and 72 (labeled with a carboxyfluorescein (FAM) at its 5′ end a black-hole quencher (BHQ-1) at its 3′ end). The decrease in fluorescence exhibited by Hel308 Mok denotes the increased processivity of these complexes as compared to Hel308 Mbu (SEQ ID NO: 10). FIG. 16 shows positive controls demonstrating that all helicases were indeed active, as denoted by a fluorescence decrease for all samples.

SEQ ID NOs: 10, 13, 16, 19, 22, 25, 28, 29, 32, 33, 34, 37, 38, 39, 40, 41, 42, 43, 44, 45, 46, 47,
48, 49, 50, 51, 52, 53, 54 and 55 (Table 4) are aligned below

```
* 1 95
10 Hel308 Mbu (1) --------------------------------MMIRELDIPRDIIGFYEDSGIKELYPPQAEAIEMGLLE-KKNLLAAIPTASGKTLLAELAMIK
53 Hel308 Afu (1) ----------------------------- MKVEELAESISSYAVGILKEEGIEELFPPQAEAVEKVFS--GKNLLLAMPTAAGKTLLAEMAMVR
22 Hel308 Csy (1) --------------------------------MRISELDIPRPAIEFLEGEGYKKLYPPQAAAAKAGLTD-GKSVLVSAPTASGKTLIAAIAMIS
75 Hel308 Dth (1) MPGVDELLQQMGQGDLQGLSTVAVKEIPAREAEFSGIEGLPPPLKQALTESGIENFYTHQARAVNLVRK--GRSVVTATPTASGKSLIYNIPVLE
48 Hel308 Fac (1) ----------------------------------MKLSEITPSEFLKVTDNNDFTLYEHQEEAVAKLREN--KNVIVSVPTASGKTLIGYISIYD
19 Hel308 Hla (1) -------------------------------MQPSSLSGLPAGVGEALEAEGVAELYPPQEAAVEAGVAD-GESLVAAVPTASGKTLIAELAMLS
55 Hel308 Hpa (1) -------------------------------MNVADLTGLPDGVPEHFHAQGIEELYPPQAEAVEAGITE-GESVVASIPTASGKTFIAELAMLS
54 Hel308 Htu (1) -------------------------------MNLEELTGLPPGATDHFRGEGIEELYPPQADAVEAGATD-GENLVAAVPTASGKTMIAALSMLS
16 Hel308 Hvo (1) -------------------------------MRTADLTGLPTGIPEALRDEGIEELYPPQAEAVEAGLTD-GESLVAAVPTASGKTLIAELAMLS
39 Hel308 Mac (1) --------------------------------MKIESLDLPDEVKRFYENSGIPELYPPQAEAVEKGLLE-GKNLLAAIPTASGKTLLAELAMLK
38 Hel308 Mba (1) --------------------------------MKIESLDLPDEVKQFYLNSGIMELYPPQAEAVEKGLLE-GRNLLAAIPTASGKTLLAELAMLK
47 Hel308 Mbo (1) --------------------------------MQIQDLAIPEPLRQQYLGLGIRELYPPQAACVERGLLD-GKNLLVAIPTASGKTLIAEMAMHR
44 Hel308 Mev (1) --------------------------------METGKLELPEYVIQFYLDTGIEKLYPPQAEAVEKGLLD-NKNLLAAIPTASGKTLISELAMLK
49 Hel308 Mfe (1) --------------------------------------MPTNKILEILKDFGIEELRPPQKKALEKGLLDKNKNFLISIPTASGKTLIGEMALIN
28 Hel308 Mfr (1) ------------------------------------DLSLPKAFIQYYKDKGIESLYPPQSECIENGLLD-GADLLVAIPTASGKTLIAEMAMHA
52 Hel308 Mhu (1) --------------------------------MEIASLPLPDSFIRACHAKGIRSLYPPQAECIEKGLLE-GKNLLISIPTASGKTLLAEMAMWS
32 Hel308 Mig (1) -------------------------------------MQKYSHVFEVLKENGIKELRPPQKKVIEKGLLNKEKNFLICIPTASGKTLIGEMALIN
51 Hel308 Min (1) -------------------------------------------MDEILKFLGIKELRPPQKKALELGILDKKKNFLISIPTGAGKTVIAEMALIN
45 Hel308 Mma (1) -----------------------------------------MHVLDLLKENKITELRPPQKKVIDEGLFDKTKNFLICIPTASGKTLIGEMALLN
40 Hel308 Mmah (1) --------------------------------MKIEELDLPSEAIEVYLQAGIEELYPPQADAVEKGLLQ-GENLLAAIPTASGKTLLAEMAMLK
76 Hel308 Mmar (1) -------------------------------MDVADLPGVPEWLPDHLRDDGIEELYPPQAEAVEAGVTE-GENLVASIPTASGKTLIAELAMLS
41 Hel308 Mmaz (1) --------------------------------MKIESLDLPDEIKRFYENSGILELYPPQAEAVEKGLLE-GKNLLAAIPTASGKTLLAELAMLK
29 Hel308 Mok (1) ----------------------------------------MLMLMEVLKENGIAELRPPQKKVVEGGLLNKNKNFLICIPTASGKTLIGEMAFIN
42 Hel308 Mth (1) -----------------------------MLTIRDLIRWLPESVIELYEALGIDELYPPQAEAIERGLLD-GRNMIISVPTAAGKTLLAELAMLR
43 Hel308 Mzh (1) --------------------------------MNINNLNLPEKVKKYYTDTGIVDLYPPQREAVDKGLLD-GENIVAAIPTASGKTLLAELCMLK
46 Hel308 Nma (1) -------------------------------MNVEELSGLPPGARSHFQEQGIEELYPPQAEAVEAGATE-GENLVAAVPTASGKTMIAALSMLS
77 Hel308 Nth (1) --------------------------------MSETFYLLSERMQKKIWEMGWDEFTPVQDKTIPIVMNT-NKDVVVSSGTASGKTEAVFLPILS
13 Hel308 Pfu (1) -----------------------------MRVDELR---VDERIKSTLKERGIESFYPPQAEALKSGILE-GKNALISIPTASGKTLIAEIAMVH
25 Hel308 Sso (1) -------------------------MSLELEWMPIEDLKLPSNVIEIIKKRGIKKLNPPQTEAVKKGLLE-GNRLLLTSPTGSGKTLIAEMGIIS
34 Hel308 Tba (1) ---------MLSTKPKAYKRFSPIG--YAMQVDELSKFGVDERIIRKIKERGISEFYPPQAEALRSGVLN-GENLLLAIPTASGKTLVAEIVMLH
33 Hel308 Tga (1) -----------------------------MKVDELP---VDERLKAVLKERGIEELYPPQAEALKSGALE-GRNLVLAIPTASGKTLVSEIVMVN
37 Hel308 Tsi (1) ---------MKLNKLKSYINAFLLGMVMSMKVDELKSLGVDERILRLLRERGIEELYPPQADALKTEVLK-GKNLVLAIPTASGKTLVAEIVMIN
50 Hel308 Mja (1) ----------------------------------------MDKILEILKDFGIVELRPPQKKALERGLLDKNKNFLISIPTASGKTLIGEMALIN
78 Consensus (1) LP V L E GI ELYPPQAEAVE GLLD GKNLLIAIPTASGKTLIAELAML
                                        96 190
      Hel308 Mbu (63) AIREGG------KALYIVPLRALASEKFERFK-ELAP----FGIKVGISTGDLDSRADWLGVNDIIVATSEKTDSLLRNGTSWMD-------EIT
      Hel308 Afu (64) EAIKGG------KSLYVVPLRALAGEKYESFK-KWEK----IGLRIGISTGDYESRDEHLGDCDIIVTTSEKADSLIRNRASWIK-------AVS
      Hel308 Csy (63) HLSRNR-----GKAVYLSPLRALAAEKFAEFGKIGGIPL-GRPVRVGVSTGDFEKAGRSLGNNDILVLTNERMDSLIRRRPDWMD-------EVG
      Hel308 Dth (94) SIINDP----ASRALYLFPLKALTRDQLTSLEEFARLLAGKVHVDSAVYDGDTDPQARARIRSKPPNILLTNPDMLHRSFLPYHRSWQKFFSALK
      Hel308 Fac (60) TYLKGK------KSMYIVPLRSLAMEKFSELL-SLRN----LGVKVTMSIGDYDVPPSFVKNYDVIIATSERADSMLHRDPDILN-------YFG
      Hel308 Hla (64) SIERGG------KALYIVPLRALASEKKTEFE-RWEE----FGVTVGVSTGNYESDGEWLATRDIIVATSEKVDSLIRNGAPWID-------DLT
      Hel308 Hpa (64) SVARGG------KALYIVPLRALASEKKEEFE-EFEQ----YGVSIGVSTGNYESDGDWLASRDIIVATSEKVDSLVRNGAKWID-------DLS
      Hel308 Htu (64) AVQRGG------KALYIVPLRALASEKKEEFE-AYEE----FGVTTGVTTGNYESTDDWLATKDIIVATSEKVDSLVRNGADWLS-------ELT
      Hel308 Hvo (64) SVARGG------KALYIVPLRALASEKKAEFE-RWEE----YGIDVGVSTGNYESDGEWLSSRDIIVATSEKVDSLVRNNAAWMD-------QLT
      Hel308 Mac (63) SVLAGG------KALYIVPLRALASEKFRRFQ-DFSE----LGIRVGISTGDYDRRDEGLGINDIIVATSEKTDSLLRNETAWMQ-------EIS
      Hel308 Mba (63) SILAGG------KALYIVPLRALASEKFRRFR-EFSE----LGIRVGISTGDYDLRDEGLGVNDIIVATSEKTDSLLRNETVWMQ-------EIS
      Hel308 Mbo (63) HIANGG------KCLYIVPLKALASEKYEEFG-NK-------GVKVGLSTGDLDRRDDALGKNDIIVATSEKVDSLLRNGARWIP-------DIT
      Hel308 Mev (63) SISNGG------KCLYIVPLRALASEKFERFK-QFSS----IGVNIGISTGDFDSTDEWLGSNDIIVATSEKADSLLRNETSWMK-------DIT
      Hel308 Mfe (58) HLLDENKNPTNKKGIFIVPLKALASEKYEEFKNKYER----YGLRVALSIGDYD-EDEDLSRYHLIITTAEKLDSLWRHKIDWID-------DVS
      Hel308 Mfr (59) AIARGG------MCLYIVPLKALATEKAQEFK-GK-------GAEIGVATGDYDQKEKRLGSNDIVIATSEKVDSLLRNGVPWLS-------QVT
      Hel308 Mhu (63) RIAAGG------KCLYIVPLRALASEKYDEFS-KKG------VIRVGIATGDLDRTDAYLGENDIIVATSEKTDSLLRNRTPWLS-------QIT
      Hel308 Mig (59) HLLDENKTPTNKKGLFIVPLKALASEKYEEFKRKYEK----YGLKVALSIGDYD-EKEDLSSYNIIITTAEKLDSLMRHEIDWLN-------YVS
      Hel308 Min (53) HLLLDK----GKKGVYIVPLKALASEKYEEFKKKYEK----FGVRVALSIGDYD-EDEDLENYDLIITTAEKFDSLWRHGIKLS--------DIS
      Hel308 Mma (55) HILDENKNLTGKKGLFIVPLKALANEKFDEFREKYEK----YGIKVGLSIGDFD-TKENLSKFHIIITTSEKLDSLMRHNVEWIN-------DVS
    Hel308 Mmah (63) AIKKGG------KALYIVPLRALASEKFRDFK-RFES----LGIKTAISTGDFDSRDEWLGSNDIIVATSEKTDSLLRNSTPWMK-------DIT
    Hel308 Mmar (64) SVARGG------KALYIVPLRALASEKQADFE-EFEQ----YGLDIGVSTGNYESEGGWLADKDIVVATSEKVDSLVRNDAPWIE-------DLT
    Hel308 Mmaz (63) SVLNGG------KALYIVPLRALASEKFRRFQ-EFSV----LGMRVGISTGDYDRRDEGLGINDIIVATSEKTDSLLRNETAWMQ-------EIS
      Hel308 Mok (56) HLLDNNKTPTNKKGLFIVPLKALANEKYEEFKGKYEK----YGLKIALSIGDFD-EKEDLKGYDLIITTAEKLDSLIRHKVEWIK-------DIS
      Hel308 Mth (66) GALSGK------RSLYIVPLRALASEKFESFS-RFSK----LGLRVGISTGDFEKRDERLGRNDIIIATSEKADSLIRNGASWVR-------RIG
      Hel308 Mzh (63) SIGMGG------KCLYIVPLKALASEKYSRFR-EFES----LGIKVGIATGDLDSREEWLGKNDIIIATSEKVDSLLRNESSWMK-------EIN
      Hel308 Nma (64) AVQRGG------KALYIVPLRALASEKKAEFD-AYEE----FGVTTGVATGNYESTSEWLATKDIIVATSEKVDSLVRNGADWLS-------DLT
      Hel308 Nth (63) QIEKDAT--KDLKILYISPLKALINDQFERIIKLCEKSY-IPIHRWHGDVNQNKKKQLTKNPAGILQITPESIESLFINRTNELNYIL---SDIE
      Hel308 Pfu (63) RILTQG-----GKAVYIVPLKALAEEKFQEFQ-DWEK----IGLRVAMATGDYDSKDEWLGKYDIIIATAEKFDSLLRHGSSWIK-------DVK
      Hel308 Sso (70) FLLKNG-----GKAIYVTPLRALTNEKYLTFK-DWEL----IGFKVAMTSGDYDTDDAWLKNYDIIITTYEKLDSLWRHRPEWLN-------EVN
      Hel308 Tba (84) KLFTGG-----GKAVYLVPLKALAEEKYREFK-TWED----LGVRVAVTTGDYDSSEEWLGKYDIIIATSEKFDSLLRHKSRWIR-------DVT
      Hel308 Tga (63) KLIQEG-----GKAVYLVPLKALAEEKYREFK-EWEK----LGLKVAATTGDYDSTDDWLGRYDIIVATAEKFDSLLRHGARWIN-------DVK
      Hel308 Tsi (86) KILREG-----GKTVYLVPLKALAEEKYKEFK-FWEK----LGIRIAMTTGDYDSTEEWLGKYDIIIATSEKFDSLLRHKSPWIK-------DIN
      Hel308 Mja (56) HLLDGNKNPTNKKGIFIVPLKALASEKYEEFKSKYER----YGLRIALSIGDYD-EDEDLSKYHLIITTAEKLDSLWRHKIDWIN-------DVS
      Consensus (96) IL GG KALYIVPLRALASEKY EFK FE GVRVGISTGDYD DEWLG DIIVATSEKVDSLLRN WI DIT
                                        191 285
      Hel308 Mbu (140) TVVVDEIHLLDSKNRGPTLEVTITKLMRLNPD----VQVVALSATVGNAREMADWLG---AALVLSEWRPTDLHEGVLFGDAINFPG-SQKKIDR
      Hel308 Afu (141) CLVVDEIHLLDSEKRGATLEILVTKMRRMNKA----LRVIGLSATAPNVTEIAEWLD---ADYYVSDWRPVPLVEGVLCEGTLELFD----GAFS
      Hel308 Csy (145) LVIADEIHLIGDRSRGPTLEMVLTKLRGLRSS----PQVVALSATISNADEIAGWLD---CTLVHSTWRPVPLSEGVYQDGEVAMGDGSRHEVAA
      Hel308 Dth (185) YIVVDEVHTYRG-VMGSNMAWVFRRLRRICAQYGREPVFIFSSATIANPGQLCSALTGHEPEVIQKGGAPAGKKHFLLLDPEMQGAAQS------
      Hel308 Fac (137) LVIIDEIHMISDPSRGPRLETVISSLLYLNPE----ILLLGLSATVSNIQEIAEWMN---AETVVSNFRAVPLETGIIFKGNLITDG--------
      Hel308 Hla (141) CVVSDEVHLVDDPNRGPTLEVTLAKLRKVNPG----LQTVALSATVGNADVIAEWLD---AELVESDWRPIDLRMGVHFGNAIDFADGSKREVPV
      Hel308 Hpa (141) CVVADEVHLVNDAHRGPTLEVTLAKLRRVNPD----LQTVALSATVGNAGEMADWLD---ATLVDSTWRPIDLRKGVLYGQALHFDDGTQQELAR
      Hel308 Htu (141) CVVSDEVHLIDDRNRGPTLEVTLAKLRRLNPG----MQVVALSATVGNADEIADWLD---ASLVDTDWRPIDLQMGVHYGNALNFDDGSTREVPV
      Hel308 Hvo (141) CVVADEVHLVDDRHRGPTLEVTLAKLRRLNTN----LQVVALSATVGNAGVVSDWLD---AELVKSDWRPIDLKMGVHYGNAVSFADGSQREVPV
      Hel308 Mac (140) VVVVDEVHLIDSADRGPTLEVTLAKLRKMNPF----CQILALSATVGNADELAAWLD---AELVLSEWRPTDLMEGVFFDGTFFCKD-KEKLIEQ
      Hel308 Mba (140) VVVADEVHLIDSPDRGPTLEVTLAKLRKMNPS----CQILALSATVGNADELAVWLE---AELVVSEWRPTELLEGVFFNGTFYCKD-REKTVEQ
      Hel308 Mbo (137) LVVIDEIHLIDSPDRGPTLEMVIAKMRSKNPG----MQLIGLSATIGNPKVLAGWLD---AELVTSSWRPVDLRQGVFYDNRIQFAE-RMRPVKQ
      Hel308 Mev (140) TIVVDEIHLLDSADRGPTLEITIAKLLRLNPN----SQIIGLSATIGNAEEIAGWLD---AELVQSQWRPIELYEGVFLEDNINFKQ-SQKPIKN
      Hel308 Mfe (141) VVVVDEIHLINDESRGGTLEILLTKLKKFN------IQIIGLSATIGNPEELANWLN---AELIVDDWRPVELKKGIYKNGIIEFINGE-----N
      Hel308 Mfr (133) CLVVDEVHLIDDESRGPTLEMVITKLRHASPD----MQVIGLSATIGNPKELAGWLG---ADLITSDWRPVDLREGICYHNTIYFDN-EDKEIPA
      Hel308 Mhu (138) CIVLDEVHLIGSENRGATLEMVITKLRYTNPV----MQIIGLSATIGNPAQLAEWLD---ATLITSTWRPVDLRQGVYYNGKIRFSD-SERPIQG
      Hel308 Mig (142) VAIVDEIHMINDEKRGGTLEVLLTKLKNLD------VQIIGLSATIGNPEELAEWLN---AELIIDNWRPVKLRKGIFFQNKIMYLNGA-----C
      Hel308 Min (131) VVVVDEIHVIGDSERGGTLEVLLTKLKELD------VQIIGLSATIGNPEELSEWLN---AELLLDNWRPVELRKGIYREGVIEYLDGE------
      Hel308 Mma (138) LAVIDEIHLIGDNERGGTLEVILTKLKNLN------AQIVGLSATIGNPEELSNWLN---AKLIVDGWRPVELKKGIYFENELEFLKNP-----A
    Hel308 Mmah (140) AVIVDEVHLLDSANRGPTLEVTLAKLKRLNPG----AQVVALSATVGNAMEIAQWLE---AKLVLSEWRPTYLHEGIFYGDAINFDE-DQTFIER
    Hel308 Mmar (141) CVVTDEVHLVDDGERGPTLEVTLAKLRRLNPD----LQTVALSATIGNAEALATWLD---AGLVDSDWRPIDLQKGVHYGQALHLEDGSQQRLSV
    Hel308 Mmaz (140) VVVADEVHLIDSPDRGPTLEITLSKLRRMNPS----CQVLALSATVGNADELAAWLD---AELVLSEWRPTDLMEGVFYNGIFYCKD-KEKPVGQ
      Hel308 Mok (139) VVVIDEIHLIGDESRGGTLEVLLTKLKTKKT-----IQIIGLSATIGNPEELAKWLN---AELIVDEWRPVKLKKGIGYGNKIMFIDDNGNTINE
      Hel308 Mth (143) VLVVDEIHLLDSANRGPTLEMTMTKLMHLNPE----MQVIGLSATIANGREIADWIK---GEIVSSDWRPVRLREGVLLEDRLVFPD-GEIQLEN
      Hel308 Mzh (140) TVVADEVHLLNSVNRGPTLEITLAKLIHLNPG----SQIIALSATIGNPEDIAGWLG---ARLVVSEWRPTDLYEGILLDGLLHIGN-IKKDIQD
      Hel308 Nma (141) CVVSDEVHLIDDRNRGPTLEVTLAKLRRLNPQ----LQVVALSATVGNADELADWLD---AELVDTDWRPIDLQMGVHYGNALNFDDGETREVPV
      Hel308 Nth (152) FIIIDELHAFLDNERGVHLRSLLSRLENYIKEK---PRYFALSATLNNFKLIKEWIN---YNDIKNVEIIDSNEDDKDLLLSLMHFDKGKDYKKP
      Hel308 Pfu (141) ILVADEIHLIGSRDRGATLEVILAHMLGKA-------QIIGLSATIGNPEELAEWLN---AELIVSDWRPVKLRRGVFYQGFVTWEDGSIDRFSS
      Hel308 Sso (148) YFVLDELHYLNDPERGPVVESVTIRAKRRN--------LLALSATISNYKQIAKWLG---AEPVATNWRPVPLIEGVIYPERKKKEYNVIFKDNT
      Hel308 Tba (162) LIVADEIHLLGSYDRGATLEMILSHMLGKA-------QILGLSATVGNAEELAEWLN---AKLVVSDWRPVKLRKGVFAHGQLIWEDGKVDKFPP
      Hel308 Tga (141) LVVADEVHLIGSYDRGATLEMILTHMLGRA-------QILALSATVGNAEELAEWLD---ASLVVSDWRPVQLRRGVFHLGTLIWEDGKVESYPE
      Hel308 Tsi (164) LVIADEIHLLGSYDRGATLEMILAHLDDKA-------QILGLSATVGNAEEVAEWLN---ADLVMSEWRPVALRKGVFYHGELFWEDGSIERFPT
      Hel308 Mja (139) VVVVDEIHLINDETRGGTLEILLTKLKEFN------VQIIGLSATIGNPDELAEWLN---AELIVDDWRPVELKKGIYKNEAIEFINGEIREIKA
      Consensus (191) VVVVDEIHLI D RGPTLEVLLAKLR LNP LQIIALSATIGNAEELAEWL AELVVSDWRPVDLR GVFY L F D I
                                        286 380
      Hel308 Mbu (227) LEK-----DDAVNLVLDTIKAEGQ-----CLVFESSRRNCAGFAKTASS---KVAKILDNDIMIKLAGIAEEVES--TGETDTAIVLANCIRKGV
      Hel308 Afu (225) TSRR----VKFEELVEECVAENGG-----VLVFESTRRGAEKTAVKLSA---ITAKYVEN------EGLEKAILE--ENEGEMSRKLAECVRKGA
      Hel308 Csy (233) TGGG-----PAVDLAAESVAEGGQ-----SLIFADTRARSASLAAKASA---VIPEAKGADAAKLAAAAKKIISS--GGETKLAKTLAELVEKGA
      Hel308 Dth (273) ----------AIRVLQKALELGLR-----TIVYTQSRKMTELIAMWASQRAGRLKKYISAYRAGFLPEQRREIEQKLASGELLAVVSTSALELGI
      Hel308 Fac (217) -EKKHLGRDDEVSLIKESIESGGQ-----ALVFRNSRRNAEKYAQSMVN----------------FFDFQNDFEKLEIPPDLFNEAQANMVAHGV
      Hel308 Hla (229) ERGE----DQTARLVADALDTEEDGQGGSSLVFVNSRRNAESSARKLTD---VTGPRLTDDERDQLRELADEIRS--GSDTDTASDLADAVEQGS
      Hel308 Hpa (229) -GNE----KETAALVRDTLEDGGS-----SLVFVNSRRNAEAAAKRLAD---VTKTHLTDDERRDLLDIADQIRD--VSDTETSDDLATAIEKGA
      Hel308 Htu (229) EGSE----KQEAALVRDILREGGS-----SLVFVNSRRNAEGAAKRLGQ---VSSREITEDERAELAELADDIRD--DSDTETSADLADCVERGA
      Hel308 Hvo (229) GRGE----RQTPALVADALEGDGEGDQGSSLVFVNSRRNAESAARRMAD---VTERYVTGDERSDLAELAAEIRD--VSDTETSDDLANAVAKGA
      Hel308 Mac (227) PTK-----DEAINLVLDTLREGGQ-----CLVFESSRKNCMGFAKKATS---AVKKTLSAEDKEKLAGIADEILE--NSETDTASVLASCVRAGT
      Hel308 Mba (227) STK-----DEAVNLALDTLKKDGQ-----CLVFESSRKNCMAFAKKAAS---TVKKTLSAEDRNALAGIADEILE--NSETDTSTNLAVCIRSGT
      Hel308 Mbo (224) VSKN----YDDLNLCLDTIAEGGQ-----CLVFVSSRRNAEAFAKRAAG---AIKSEDA-----ALAACAERLLE--GTPTEMVKTLAACVAKGA
      Hel308 Mev (227) IVK-----DTAVNLVLDTIDENGQ-----CLVFESSRRNCAGFAKKAKS---KVGKSLDKGLLAELNNIAEEVLE--TSDTETTKELASCIKRGT
      Hel308 Mfe (222) REIKAINNNDIYNLVVDCVKDGGC-----CIVFCNTKRGAVNEAKKLN-----LKKFLTNEEKRKLKEVAEEILSILEPPTEMCKTLAECILNGS
      Hel308 Mfr (220) PAK-----TEDINLLLDCVADGGQ-----CLVFVSSRRNAEGYAKRAAT---ALKCSHA-----ALDSIAEKLEA--AAETDMGRVLATCVKKGA
      Hel308 Mhu (225) KTK-----HDDLNLCLDTIEEGGQ-----CLVFVSSRRNAEGFAKKAAG---ALKAGSP-----DSKALAQELRR--LRDRDEGNVLADCVERGA
      Hel308 Mig (223) KELPNFSNNPMLNLVLDCVKEGGC-----CLVFCNSKNGAVSEAKKLN-----LKKYLSNSEKYELQKLKEEILSILDPPTETCKTLAECLEKGV
      Hel308 Min (211) -------VKECQDIVKEVVKDNGS-----VIIFCPTKKKAENRALSLD-----LSDLLKKSEKRKLEEISEELLSLFDPPTELCKKLASCVRKGI
      Hel308 Mma (219) KKIKQVSRNNLTDLIVDSVEEKGS-----CLIFCNSKRNAVGEAKKHN-----LAKYLTRTEQHELNKLSEEILSILDRPVETCKALSKCIQNGV
    Hel308 Mmah (227) RHK-----EDSVNLVIDTVIQGGQ-----CLVFDSSRRNCVGFAKKCAP---AVGELLDRQNRNELEEVAKEVLE--NGETKLTETLAYCIKKGV
    Hel308 Mmar (229) QNNE----KQTAAIVRDTLEDDGS-----TLVFVNSRRNAEAAAGRLAN---TVRPHLSTEERDQLADIAEEIRD--VSDTETSDDLADAVADGA
    Hel308 Mmaz (227) PTK-----DEAVNLVLDTIKEGGQ-----CLVFESSRKNCMGFAKKAVS---AVKKTLSNEDRETLAGIADEIIE--NSETDVSSVLATCVRSGT
      Hel308 Mok (226) VIVDEISKNNMFNLVVDSILKDGS-----CIIFCNSKRGAVGEAKKLN-----LKKYLSPDEISELRHLKEEVLSVLDNPTKTCKDLAECIEKGV
      Hel308 Mth (230) RNR-----DPVLNLVLDTVDQGGQ-----MLIFESTRRNAESMAKKVSG---ALQESGE------TIELAERLS----GEGKTAKKLAMCLRHGA
      Hel308 Mzh (227) ESR-----DDAVNLVIDTVKDKGQ-----CLVFESSRRNCMGFAKKAGK---WVSKILDEHDTIQLKSLSQEIGE--AGETEIADVLSRCVRQGV
      Hel308 Nma (229) EAGE----KQEAALVRDILQEGGS-----SLVFVNSRRNAEAAARRLGQ---VSSRELTAGEQNDLAALATEIRE--DSDTETSQDLADCVERGA
      Hel308 Nth (241) ----------ID-LYQDLRELTKN---VHSLIFCNSRAEVEETTLYLNR---LANREVNTELYLAHHSSIDKKER-EYVEKTMANSKSPKSVVTT
      Hel308 Pfu (226) ----------WEELVYDAIRKKKG-----ALIFVNMRRKAERVALELSK---KVKSLLTKPEIRALNELADSLE----ENPTNEKLAKAIRGGV
      Hel308 Sso (232) TKKVHG-DDAIIAYTLDSLSKNGQ-----VLVFRNSRKMAESTALKIAN---YMNFVSLDEN--ALSEILKQLDDIEEGGSDEKELLKSLISKGV
      Hel308 Tba (247) Q---------WDSLVIDAVKKGKQ-----ALVFVNTRRSAEKEAGMLGK---KVRRLLTKPEARRLKELAESLE-----SNPTNDKLKEVLVNGA
      Hel308 Tga (226) N---------WYSLVVDAVKRGKG-----ALVFVNTRRSAEKEALALSK---LVSSHLTKPEKRALESLASQLE-----DNPTSEKLKRALRGGV
      Hel308 Tsi (249) Q---------WDSLVIDALKKGKQ-----ALVFVNTRRSAEKEALLLAG---KIQRFLTKPEERKLKQLADGLD-----TTPTNQKLKEALTKGV
      Hel308 Mja (225) VDN-----NDIYNLVVDCVKEGGC-----CLVFCNTKRNAVNEAKKLN-----LKKFLTEEEKIRLKEIAEEILSILEPPTEMCKTLAECILNGS
      Consensus (286) LVLDTV EGGQ LVF NSRRNAE AKKLA V K LT E L LAEEI ETETS LA CV KG
                                        381 475
      Hel308 Mbu (307) AFHHAGLNSNH-----------RKLVENGFRQNLIKVISSTPTLAA-------------------------------------------------
      Hel308 Afu (300) AFHHAGLLNGQ-----------RRVVEDAFRRGNIKVVVATPTLAA-------------------------------------------------
      Hel308 Csy (313) AFHHAGLNQDC-----------RSVVEEEFRSGRIRLLASTPTLAA-------------------------------------------------
      Hel308 Dth (353) DIGHLDLCLLVGYPGSVMATMQRGGRVGRSGRDSAIMLIGHEDALDQYLLRNPREFFSLEPESAVINPDNPSIMRRHLVCAAAEKPIALQEMMLD
      Hel308 Fac (290) MFHHAGLSNDQ-----------RTMIEKLFKQGYIKILTATPTLAA-------------------------------------------------
      Hel308 Hla (315) AFHHAGLRSED-----------RARVEDAFRDRLIKCISATPTLAA-------------------------------------------------
      Hel308 Hpa (309) AFHHAGLASDH-----------RSLVEDAFRDKLIKVISATPTLAA-------------------------------------------------
      Hel308 Htu (310) AFHHAGLSSTQ-----------RSLVEDAFRDRLLKVISATPTLAA-------------------------------------------------
      Hel308 Hvo (315) AFHHAGLAAEH-----------RTLVEDAFRDRLIKCICATPTLAA-------------------------------------------------
      Hel308 Mac (307) AFHHAGLTSPL-----------RELVETGFREGYVKLISSTPTLAA-------------------------------------------------
      Hel308 Mba (307) AFHHAGLTTPL-----------RELVEDGFRAGRIKLISSTPTLAA-------------------------------------------------
      Hel308 Mbo (300) AFHHAGLSRKE-----------RSIVEEAFRKNLLKCISSTPTLAA-------------------------------------------------
      Hel308 Mev (307) AFHHAGLNSAQ-----------RKIVEDNFRNNKIKVISSTPTLAA-------------------------------------------------
      Hel308 Mfe (307) AFHHAGLTYQH-----------RKIVEDAFRNKLIKVICCTPTLSV-------------------------------------------------
      Hel308 Mfr (295) AFHHAGMNRMQ-----------RTLVEGGFRDGFIKSISSTPTLAA-------------------------------------------------
      Hel308 Mhu (300) AFHHAGLIRQE-----------RTIIEEGFRNGYIEVIAATPTLAA-------------------------------------------------
      Hel308 Mig (308) AFHHAGLTYEH-----------RKIVEEGFRNKLIKVICCTPTLSA-------------------------------------------------
      Hel308 Min (289) AFHHSGLTYEH-----------RKIIEKAFRERILKVICSTTTLAF-------------------------------------------------
      Hel308 Mma (304) AFHHAGLTYKH-----------RKIVEDGFRNRLIKVICCTPTLSA-------------------------------------------------
    Hel308 Mmah (307) AFHHAGLNSAH-----------RRIVEDAFRNNLIKMICSTPTLAA-------------------------------------------------
    Hel308 Mmar (310) AFHHAGLSRGH-----------RELVEDAFRDRLVKVVCATPTLAA-------------------------------------------------
    Hel308 Mmaz (307) AFHHAGLTTPL-----------RELVENGFREGRIKIISSTPTLAA-------------------------------------------------
      Hel308 Mok (311) AFHHAGLTYEQ-----------RKIVEEGFRKKLIKAICCTPTLSA-------------------------------------------------
      Hel308 Mth (302) AFHHAGLLPEQ-----------RRLIELGFRQNVVKVIACTPTLAA-------------------------------------------------
      Hel308 Mzh (307) AFHHAGLNSEH-----------RRMVEEGFRKNLIKMISSTPTLAA-------------------------------------------------
      Hel308 Nma (310) AFHHAGLSSTQ-----------RSLVEDAFRDRLLKVISATPTLAA-------------------------------------------------
      Hel308 Nth (318) SSLELGIDIGA-----------IDYVVQIDDTHTVSSLKQRLGRSG-------------------------------------------------
      Hel308 Pfu (298) AFHHAGLGRDE-----------RVLVEENFRKGIIKAVVATPTLSA-------------------------------------------------
      Hel308 Sso (316) AYHHAGLSKAL-----------RDLIEEGFRQRKIKVIVATPTLAA-------------------------------------------------
      Hel308 Tba (320) AFHHAGLGRAE-----------RTLIEDAFREGLIKVLTATPTLAM-------------------------------------------------
      Hel308 Tga (299) AFHHAGLSRVE-----------RTLIEDAFREGLIKVITATPTLSA-------------------------------------------------
      Hel308 Tsi (322) AFHHAGLGRTE-----------RSIIEDAFREGLIKVITATPTLSA-------------------------------------------------
      Hel308 Mja (305) AFHHAGLTYQH-----------RKIVEDAFRKRLIKVICCTPTLSA-------------------------------------------------
      Consensus (381) AFHHAGL R LVEDAFR LIKVI ATPTLAA
                                        476 570
      Hel308 Mbu (342) --------------------------------------GLNLPARRVIIRSYRRFDS-NFG--------------------------MQPIPVLE
      Hel308 Afu (335) --------------------------------------GVNLPARRVIVRSLYRFDG-YSK----------------------------RIKVSE
      Hel308 Csy (348) --------------------------------------GVNLPARRVVISSVMRYNS-SSGM-------------------------SEPISILE
      Hel308 Dth (448) NEAGKCIKSLEKDGELLASRDRSFYYTRARYPHKDVDLRGIGQTYNIFEHSTGEYLGEVDGVRAFKETHPGAVYLHMGETYVVQDLDLETFAVYA
      Hel308 Fac (325) --------------------------------------GVNLPARTVIIRDITRFSD---GY-------------------------SKPISGIE
      Hel308 Hla (350) --------------------------------------GVNTPARRVIVRDWRRYDG-EFGG-------------------------MKPLDVLE
      Hel308 Hpa (344) --------------------------------------GVNTPSRRVIVRDWRRYDG-DIGG-------------------------MQPLDVLE
      Hel308 Htu (345) --------------------------------------GVNTPARRVIVRDWRRFDP-SAGG-------------------------MAPLDVLE
      Hel308 Hvo (350) --------------------------------------GVNTPSRRVVVRDWQRYDG-DYGG-------------------------MKPLDVLE
      Hel308 Mac (342) --------------------------------------GLNLPARRVIIRSYRRYSS-DSG--------------------------MQPIPVLE
      Hel308 Mba (342) --------------------------------------GLNLPARRVIIRNYRRYSS-EDG--------------------------MQPIPVLE
      Hel308 Mbo (335) --------------------------------------GLNLPARRVIIRDYLRFSA-GEG--------------------------MQPIPVSE
      Hel308 Mev (342) --------------------------------------GLNLPARRVIVRNYKRYDP-NFG--------------------------MQPIPVLD
      Hel308 Mfe (342) --------------------------------------GLNLPCRRAIVKDLTRYT--NRG--------------------------MRYIPIME
      Hel308 Mfr (330) --------------------------------------GLNLPARRVIIRDYLRYSG-GEG--------------------------MRPIPVRE
      Hel308 Mhu (335) --------------------------------------GLNLPARRVIIRDYNRFAS-GLG--------------------------MVPIPVGE
      Hel308 Mig (343) --------------------------------------GINIPCRRAIVRDLMRFS--NGR--------------------------MKPIPIME
      Hel308 Min (324) --------------------------------------GLNLPCRRVIISELKRYT--RRG--------------------------LTYIPIME
      Hel308 Mma (339) --------------------------------------GLNLPCRRAIVRDIKRYS--QNG--------------------------LVDIPRME
    Hel308 Mmah (342) --------------------------------------GLNLPARRVIIRSYKRYDP-NAG--------------------------MQPIPVLD
    Hel308 Mmar (345) --------------------------------------GVNTPSRRVVVRDWRRYDG-SAGG-------------------------MAPLSVLE
    Hel308 Mmaz (342) --------------------------------------GLNLPARRVIIRSYRRYSS-DSG--------------------------MQPIPVLE
      Hel308 Mok (346) --------------------------------------GINMPCRRAIIRDLKRFS--SRG--------------------------YIPIPKME
      Hel308 Mth (337) --------------------------------------GLNLPARRVLIRSYKRYEA-GLG--------------------------TRPIPVME
      Hel308 Mzh (342) --------------------------------------GLNLPARRVIIRSYKRYDP-NFG--------------------------MKPIPVLE
      Hel308 Nma (345) --------------------------------------GVNTPARRVIVRDWRRFDP-SAGG-------------------------MAPLDVLE
      Hel308 Nth (353) --------------------------------------RKLGINQVLQVYSTINDSLVQSLA-------------------------VIDLLLEK
      Hel308 Pfu (333) --------------------------------------GINTPAFRVIIRDIWRYS--DFG--------------------------MERIPIIE
      Hel308 Sso (351) --------------------------------------GVNLPARTVIIGDIYRFNKKIAGY-------------------------YDEIPIME
      Hel308 Tba (355) --------------------------------------GVNLPSFRVIIRDTKRYS--TFG--------------------------WSDIPVLE
      Hel308 Tga (334) --------------------------------------GVNLPSFRVIIRDTKRYA--GFG--------------------------WTDIPVLE
      Hel308 Tsi (357) --------------------------------------GVNLPAYRVIIRDTKRYS--NFG--------------------------WVDIPVLE
      Hel308 Mja (340) --------------------------------------GLNLPCRRAIVKDLTRFTN---KG-------------------------MRYIPIME
      Consensus (476) GLNLPARRVIIRDYKRY G M PIPVLE
                                        571 665
      Hel308 Mbu (372) YKQMAGRAGRPHLDPYGESVLLAKTYDEF--AQLMENYVEADAEDIWSKLGTENALRTHVLSTIVNGFASTRQELFDFFGATFFAYQQ-DKWMLE
      Hel308 Afu (363) YKQMAGRAGRPGMDERGEAIIIVGKRDR---EIAVKRYIFGEPERITSKLGVETHLRFHSLSIICDGYAKTLEELEDFFADTFFFKQN--EISLS
      Hel308 Csy (379) YKQLCGRAGRPQYDKSGEAIVVGGVNAD----EIFDRYIGGEPEPIRSAMVDDRALRIHVLSLVTTSPGIKEDDVTEFFLGTLGGQQS-GESTVK
      Hel308 Dth (543) AKSEANYYTRPITEKYTEIVEVQATRATAAGELCLGRLKVTEHVSAYEKRLVRGQARIGLIPLDLPPLVFETQGMWFTLDSQVRRDVEDRRLHFM
      Hel308 Fac (354) IQQMIGRAGRPKYDKKGYGYIYAASPG---MLRVAEGYLTGELEPVISRMDSNSLIRFNVLALISSGIATDLKGIQDFYGKTLLAAQN-DIDGYE
      Hel308 Hla (381) VHQMCGRAGRPGLDPYGEAVLLANDADTK--EELFERYLWADPEPVRSKLAAEPALRTHVLATVASGFASTRDGLLSFLDNTLYATQTDDEGRLA
      Hel308 Hpa (375) VHQMFGRAGRPGLDPHGEAVLIAKSHDEL--QELFDQYVWADPEPVHSKLAAEPALRTHILATVASGFAGTEEELLDFLERTLYATQTDETGRLE
      Hel308 Htu (376) VHQMMGRAGRPGLDPYGEAVLLAKSHDES--EELFDRYIWADPEPVRSKLAAEPALRTHVLATIASGFARTRGGLLEFLEATLYASQSSEAGRLE
      Hel308 Hvo (381) VHQMMGRAGRPGLDPYGEAVLLAKDADAR--DELFERYIWADAEDVRSKLAAEPALRTHLLATVASGFAHTREGLLEFLDQTLYATQTDDPERLG
      Hel308 Mac (372) YKQMAGRAGRPRLDPYGEAVLLAKSYEEL--LFLFEKYIEAGAEDIWSKLGTENALRTHVLSTISNGFARTKEELMDFLEATFFAYQY-SNFGLS
      Hel308 Mba (372) YKQMAGRAGRPRLDPYGEAVLVAKSYKEF--VFLFENYIEANAEDIWSKLGTENALRTHVLSTISNGFARTYDELMDFLEATFFAFQY-SNFGLS
      Hel308 Mbo (365) YRQMAGRAGRPRLDPYGEAVLIAKEAEQV--PELFEVYIEAEAEDVHSRIAEPTALYTHVLSLVASGFAGTRGELTEFMNRSFYVHEHKQGRLIH
      Hel308 Mev (372) YKQMAGRAGRPSLDPYGESVLISHTYNEF--TDLLDRYIDAEPEDILSKLGTENALRTHVLSTIVNGFATTRQGMVDFMGSSFFAYQQ-QKWSLI
      Hel308 Mfe (371) IQQCIGRAGRLGLDPYGEGIIVAKNDR---DYLRSYQVLTQKPEPIYSKLSNQAVLRTQLLGLIATIEIRDEYDLEWFIRNTFYAYQYGNLREVA
      Hel308 Mfr (360) YRQMAGRAGRPHLDPYGEAILIAKTEYAV--NDLHEEYVEAPDEDVTSRCGEKGVLTAHILSLIATGYARSYDELMAFLEKTLYAYQHTGKKALT
      Hel308 Mhu (365) YHQMAGRAGRPHLDPYGEAVLLAKDAPSV--ERLFETFIDAEAERVDSQCVDDASLCAHILSLIATGFAHDQEALSSFMERTFYFFQHPKTRSLP
      Hel308 Mig (372) IHQCIGRAGRPGLDPYGEGIIFVKNER---DLERAEQYLEGKPEYIYSKLSNQAVLRTQLLGMIATREIENEFDLISFIKNTFYAHQYGNLGGVL
      Hel308 Min (353) VQQCIGRAGRPGLDEYGEGILVAKDER---DYLRALQCLTQKPEPIYSKLSNDSVLRTQILGLIATRYVLDEYDLEEFIKNTFYAYQYKNLDEIK
      Hel308 Mma (368) IQQCIGRAGRPGLDPYGEGIIYIKNER---DAEKAYEILTGSVENIYSKLANQKVLRIHILGLISTGEIKDGQNLVNFMKNTFYAHQFGNIGAVL
    Hel308 Mmah (372) YKQMAGRAGRPHLDPYGEAVVIVKTYEEF--TDVLERYISASAEDIWSKLGTENALRTHILSTIASGFANCHREILTFLGSTFFAHQQ-QSWNFE
    Hel308 Mmar (376) VHQMMGRAGRPGLDPYGEAVLIASSHDEV--DELFERYVWADPEPVRSKLAAEPALRTHILATVASGFARSRKGLLEFLEQTLYASQTDDSGQLE
    Hel308 Mmaz (372) YKQMAGRAGRPRLDPYGEAVLLAKSYEEF--VFLFEKYIEAGAEDIWSKLGTENALRTHILSTISNGFARTREELMDFLEATFFAFQY-SNFGLS
      Hel308 Mok (375) IHQCIGRAGRPNLDPYGEGIIYINNTENPELIENAKNYLIGNVEEIYSKLSNQKVLRTHMLGLITTGDIKNKNDLEEFIKNTFYAYQYQNTKKIL
      Hel308 Mth (367) YRQMAGRAGRPGLDPYGESLIMARSESEL--QKLMDHYVMGEPEDIWSKLASERALRTHVLATIASRFADSVDSLSRLMASTFYARQQ-DPSYLG
      Hel308 Mzh (372) YKQMAGRAGRPHLDPYGESVLIARSYDEF--MDIMENYVNADPEDIWSKLGTENALRTHVLSTIVNGFAYTYRGLMDFVKMTFFAYQK-EASDLH
      Hel308 Nma (376) VHQMMGRAGRPGLDPYGEAVLLAKSHDES--QELFDRYVWADPEPVRSKLAAEPALRTHVLATIASGFARTREGLLEFLEATLYASQSSEGGRLE
      Hel308 Nth (385) WIEPATEYPLPLDILFHQIISICHEANGVRLDPLIDNIKANAAFYKLKEEDINHVINYMIENDFLQLIRNSAELIVGLEGERLLRGKEFYAVFMT
      Hel308 Pfu (362) VHQMLGRAGRPKYDEVGEGIIVSTSDD---PREVMNHYIFGKPEKLFSQLSNESNLRSQVLALIATFGYSTVEEILKFISNTFYAYQRKDTYSLE
      Hel308 Sso (383) YKQMSGRAGRPGFDQIGESIVVVRDKEDV--DRVFKKYVLSDVEPIESKLGSERAFYTFLLGILSAEGNLSEKQLENFAYESLLAKQL-----VD
      Hel308 Tba (384) IQQMIGRAGRPKYDKEGEAIIVAKTEK---PEELMEKYIFGKPEKLFSMLSNDAAFRSQVLALITNFGVESFRELIGFLEKTFYYHQRKDLEILE
      Hel308 Tga (363) IQQMMGRAGRPRYDKYGEAIIVARTDE---PGKLMERYIRGKPEKLFSMLANEQAFRSQVLALITNFGIRSFPELVRFLERTFYAHQRKDLSSLE
      Hel308 Tsi (386) IQQMMGRAGRPKYDIEGQAIIIAKTEK---PEDLMKRYVLGKPEKLFSMLSNEASFRSQVLALITNFGVGNFKELVNFLERTFYYHQRKNLEALE
      Hel308 Mja (369) IQQCIGRAGRPGLDPYGEGIIVAKNDRDY---LRAYQALTQKPEPIYSKLSNQAVLRTQLLGLIATGEIRDEYDLEWFIRNTFYAHQYGNLREVA
      Consensus (571) I QM GRAGRP LDPYGEAVLIAKS D EL E YI ADPE IWSKLA E ALRTHVLALIASGFA T ELLDFL TFYAYQ L
                                        666 760
      Hel308 Mbu (464) EVINDCLEFLIDKAMVSET-E--------------------------------------DIEDASKLFLRGTRLGSLVSMLYIDPLSGSKIVDGF
      Hel308 Afu (453) YELERVVRQLENWGMVVEAAH-----------------------------------------------LAPTKLGSLVSRLYIDPLTGFIFHDVL
      Hel308 Csy (469) FSVAVALRFLQEEGMLGRR----------------------------------------------GGRLAATKMGRLVSRLYMDPMTAVTLRDAV
      Hel308 Dth (638) GGLHALEHGLIGCMPLIILTDRNDLGGIASPVHEQLHKG--------------AVFIYDGTPGGIGLCRQAFELGDRLVARAMGILSSCTCENGC
      Hel308 Fac (445) LAFESALYFLKDNDFITEEN----------------------------------------------DIYSATKFGRLTSDLYIDPVSSLILKKCL
      Hel308 Hla (474) AVTDTVLDYLAVNDFIERDRD------------------------------------------GGSESLTATGIGHTVSRLYLDPMSAAEMIDGL
      Hel308 Hpa (468) TVTQHVLDYLDRNGFLERDD-----------------------------------------------RLRATGLGHRVSQLYLDPMSAAEIIDGL
      Hel308 Htu (469) SVTDDVLDYLERNDFIERSR--DDEAEDSGEDDGPFTSAADLAEQ---------QAAK------REETLEATSLGHTVSRLYLDPMSAAEIVHGL
      Hel308 Hvo (474) QVTDRVLDYLEVNGFVEFEG----------------------------------------------ETIQATPVGHTVSRLYLDPMSAAEIIDGL
      Hel308 Mac (464) VVVDECLNFLRQEGMLEQDS----------------------------------------------DALISTMFGKLVSRLYIDPLSAALIAKGL
      Hel308 Mba (464) TVVNECLNFLRQEGMLEKD-----------------------------------------------DALIPTSFGKLVSRLYIDPLSAARIAKGL
      Hel308 Mbo (458) RAIDEALQFLITAEMVVEV----------------------------------------------GEHIGATELGTLVSRMYIDPRSAFAIVTTL
      Hel308 Mev (464) DVVDDCIEFLQDNEMIKD-----------------------------------------DG--ER---LYATRLGQVISTLYIDPLSGAIIIDKL
      Hel308 Mfe (463) KNINEVIRFLEEK--------------------------------------------------EFMIDFIPTELGKRVAELYIDPLSAKYMIDGL
      Hel308 Mfr (453) RTLDDALGFLTEAEMVTDL----------------------------------------------SGMLHATEYGDLTSRLYIDPHSAEIITTAL
      Hel308 Mhu (458) RLVADAIRFLTTAGMVEER----------------------------------------------ENTLSATRLGSLVSRLYLNPCTARLILDSL
      Hel308 Mig (464) RNIKEVINFLEEN--------------------------------------------------DFIADYFPTKLGKRVSELYIDPLSAKIIIDGL
      Hel308 Min (445) KKIKEIIEFLEDCN--------------------------------------------------FIKNFEVTPLGKKVSNLYLDPLSAKIMIDNI
      Hel308 Mma (460) LNVSEVVEFLEKNKFLETTIHKKTENKVRELSFDS------S-NN---LVLDSKETSFDLTNPNSNIEFRSTKLGKRISELYIDPMSSEIIIEEL
    Hel308 Mmah (464) ELLEDCLIFLKNEGMLEQD-N--------------------------------------ET-------IRATELGKMISSLYIDPLSASKIIRGL
    Hel308 Mmar (469) RVVDDVLTYLQRNDFLEIEAG----------------------------------------------ELDATSLGHTVSRLYLDPMSAAEIVDGL
    Hel308 Mmaz (464) AVVDECLDFLRREGMLEKDP----------------------------------------------DALVSTVFGKLVSRLYIDPLSAALIAKGL
      Hel308 Mok (470) ENIYEITNFLEKNGFIELNYRRDENKDKSNNSHNNKKNISNTNNSIKMLVLDNNNSLTIKSRHEEDVYYNITPLGKKVSELYIDPLSAEYIIDGL
      Hel308 Mth (459) ETIASVLEFLVRSDMIDKD-------------------------------------------------LTPTPLGALVSRLYIDPLSAMVMIQEI
      Hel308 Mzh (464) DVIEECVRFLIDNEMIISD-S--------------------------------------NDILPES-AFRSTATGKLISMLYIDPLSGSLIMDGI
      Hel308 Nma (469) RVTDDVLSYLERNDFIERSGGPEDTLNSEADAASAFTSAADLADS---------DGGDSGGTTGQEEDLEATSLGHTVSRLYLDPMSAAEIVHGL
      Hel308 Nth (480) QEEFEVREGIRKIGSIDKS-------------------------------------------LMVSEGDNIILAGQLWTIKNIDIERDIIYVAKA
      Hel308 Pfu (454) EKIRNILYFLLEN---------------------------------------------EFIEISLEDKIRPLSLGIRTAKLYIDPYTAKMFKDKM
      Hel308 Sso (471) VYFDRAIRWLLEHSFIKEE----------------------------------------------GNTFALTNFGKRVADLYINPFTADIIRKGL
      Hel308 Tba (476) GKAKSIVYFLLEN---------------------------------------------EFIDIDLNDSFIALPFGIRTSQLYLDPLTAKKFKDAL
      Hel308 Tga (455) YKAKEVVYFLIEN---------------------------------------------EFIDLDLEDRFIPLPFGKRTSQLYIDPLTAKKFKDAF
      Hel308 Tsi (478) GKAKSIVYFLFEN---------------------------------------------EFIDIDLNDQFMPLPLGIRTSQLYLDPVTAKKFKDAF
      Hel308 Mja (461) KNINEVIRFLEENEFI--------------------------------------------------IDFMPTELGKRVSELYIDPLSAKFIIDGL
      Consensus (666) I EVL FL N I L AT LG VS LYIDPLSA IIDGL
                                        761 855
      Hel308 Mbu (520) KDIGKSTGGNMGSLEDDKG----------------------------------------------------DDITVTDMTLLHLVCSTPDMRQLY
      Hel308 Afu (501) SRMELS-----------------------------------------------------------------------DIGALHLICRTPDMERLT
      Hel308 Csy (518) GEASPGR--------------------------------------------------------------------MHTLGFLHLVSECSEFMPRF
      Hel308 Dth (719) PGCIHSPKCGSGNR------------------------------------------------------------PLDKEAAMHMLAVLAGERCGE
      Hel308 Fac (494) DLEFS-------------------------------------------------------------------------ELYLYYISKTPDMLTFN
      Hel308 Hla (527) RSVARDAADTGASAEADNG-EFVRTGDADDASGGDEPGFGTYTRAGDDESGER-------TENEETDEEETEASEVTPLGLYHLISRTPDMYELY
      Hel308 Hpa (516) RDADG---------------------------------------------------------------------KPTALGLYHLVSRTPDMYQLY
      Hel308 Htu (547) ERADER---------------------------------------------------------------------PTALGLYQLVSRTPDMYELY
      Hel308 Hvo (523) EWAADHRTEKLRALAGETPEKPTRDRSESDESGGFQRASEMVADDGDGGGGEDGVGANGDGDSDDADGVETDRTYPTPLGLYHLVCRTPDMYQLY
      Hel308 Mac (513) REAGT----------------------------------------------------------------------LTELTLLHLVCSTPDMRLMY
      Hel308 Mba (512) KGAKS----------------------------------------------------------------------LSELTLLHLVCSTPDMRLLY
      Hel308 Mbo (507) REQEK----------------------------------------------------------------------YADLGLIQLICTTPDMPTLY
      Hel308 Mev (513) KKADK-----------------------------------------------------------------------TDMTMLHIICSTPDMRQLY
      Hel308 Mfe (508) NEMENED----------------------------------------------------------------------DIYYLYLISKTLEMMPNL
      Hel308 Mfr (502) REEGE-----------------------------------------------------------------------TDLALLQLLCMTPDMFTLY
      Hel308 Mhu (507) KSCKT----------------------------------------------------------------------PTLIGLLHVICVSPDMQRLY
      Hel308 Mig (509) KEMGNVDNE--------------------------------------------------------------------ELYYLYLISKTLEMMPLL
      Hel308 Min (490) EVKDDLH-------------------------------------------------------------------------LLYILCKCIEMKPLL
      Hel308 Mma (545) HELKKKCDQLDR--------------------------------------------------------------SKIDQYLFYLISKTNEMRPLL
    Hel308 Mmah (513) EKTTH----------------------------------------------------------------------VTDMTLLQLICSTPDMRLLY
    Hel308 Mmar (518) RDWERGASDSTSASGSPAD----AQAEP-PANSGFTTASELAEDADESDADRD--------------------DDISALGLYHLVSRTPDMYQLY
    Hel308 Mmaz (513) REAGT----------------------------------------------------------------------LTELTLLHLICSTPDMRLMY
      Hel308 Mok (565) KNLHKKTLSNPKNM------------------------------------------------------------ECYILHILYIISKTTEMQPVL
      Hel308 Mth (505) RGIRR----------------------------------------------------------------------PTVLTLLHVITMTPDMELLF
      Hel308 Mzh (519) RKADY----------------------------------------------------------------------FEDITMMHLICSTPDMKNLY
      Hel308 Nma (555) EDADER---------------------------------------------------------------------PTALGLYQLVSRTPDMYELY
      Hel308 Nth (532) VDGKPPK--------------------------------------------------------------------YSGGGFILNPKIPERMHKIL
      Hel308 Pfu (504) EEVVKDPN---------------------------------------------------------------------PIGIFHLISLTPDITPFN
      Hel308 Sso (520) EGHKAS----------------------------------------------------------------------CELAYLHLLAFTPDGPLVS
      Hel308 Tba (526) PQIEENPN---------------------------------------------------------------------PLGIFQLLASTPDMGTLS
      Hel308 Tga (505) PAIERNPN---------------------------------------------------------------------PFGIFQLIASTPDMATLT
      Hel308 Tsi (528) EKLEKNPN---------------------------------------------------------------------PLGIFQLLASTPDMSSLR
      Hel308 Mja (506) EEMENEE----------------------------------------------------------------------EIYYLYLISKTLEMMPNL
      Consensus (761) LGLLHLIS TPDM LY
                                        856 950
      Hel308 Mbu (563) LRNTDYTIVNEYIVAHSDEFH---EIPDKLKETDYEWFMGEVKTAMLLEEW---------------VTEVSAEDITRHFNVGEGDIHALADTSEW
      Hel308 Afu (525) VRKTDSWVEEEAFRLRKELSY----YPSDFS-VEYDWFLSEVKTALCLKDW---------------IEEKDEDEICAKYGIAPGDLRRIVETAEW
      Hel308 Csy (545) ALRQKDHEVAEMMLEAGRGELLR---P--------VYSYECGRGLLALHRW---------------IGESPEAKLAEDLKFESGDVHRMVESSGW
      Hel308 Dth (754) AKRKDVSCRIETDEGSMEIDSG-YTKSDQAELPYAVLDIETRYSAQEVGGWGNCHRMGVSFAVVFDSRNQEFVTFDQEQAADLGSFLEDFSLVVG
      Hel308 Fac (517) YRASDYEYLEEFLDRHNISDFS-------------EESMGAAKTAIILNEW---------------INEVPINTIAETFGIGPGDIQAKASSADW
      Hel308 Hla (615) LKSGDRETYTELCYERETEFLG--DVPSEYEDVRFEDWLASLKTARLLEDW---------------VNEVDEDRITERYGVGPGDIRGKVDTAEW
      Hel308 Hpa (542) LRSGDRERYTEIAYEREPEFLG--HMPSEFEDNAFEDWLSALKTARLLEDW---------------ASELDEDRITERYAIGPGDIRGKVETAQW
      Hel308 Htu (573) LRSGEDEKFGELFYERETELLG--DAPSEYEEDRFEDWLAALKTGKLLEDW---------------ADETDEETITDRYKIGPGDLRGKVDTAEW
      Hel308 Hvo (618) LKSGDRETYTELCYEREPEFLG--RVPSEYEDVAFEDWLSALKTAKLLEDW---------------VGEVDEDRITERYGVGPGDIRGKVETSEW
      Hel308 Mac (538) MRSQDYQDINDFVMAHAEEFS---KVPSPFNIVEYEWFLSEVKTSLLLMDW---------------IHEKPENEICLKFGTGEGDIHTTADIAEW
      Hel308 Mba (537) MRSHDYQDINDYVMAHASEFV---KVPSPFDTTEYEWFLGEVKTSLLLLDW---------------IHEKSENEICLKFGTGEGDIHSIADIAEW
      Hel308 Mbo (532) AKNADLPALSRMLEVRGADIW---LPP-PLDDDAAETYYRAVKTAMLLSDW---------------TDELSEEKICERYGVGPGDVFGMVENINW
      Hel308 Mev (538) LRSKEYEKINEYVMTHSDEFV---EVPNPFKSIEYEWFLGEVKTALLINEW---------------IDEKTLDDITAEFGVGEGDINALSDISEW
      Hel308 Mfe (533) RVYKSEE--LNLIDEMENLG------IKSFE----IEDLEAFKTAKMLYDW---------------ISEVPEDEILKKYKIEPGILRYKVENAVW
      Hel308 Mfr (527) VKKNDLGTLEKFFFEHEEEFR---T---EFSYDEMEDFFRSLKTAMLLSDW---------------TDEIGDDTICTRFGVGPGDIFNAVQGISW
      Hel308 Mhu (532) LKAADTQLLRTFLFKHKDDLI---LPL-PFEQEEEELWLSGLKTALVLTDW---------------ADEFSEGMIEERYGIGAGDLYNIVDSGKW
      Hel308 Mig (536) RVNSFEE--LDLILEMEEAG------IYDRT----YDDLAAFKNAKMLYDW---------------INEVPEDEILKKYKIEPGILRYKVEQAKW
      Hel308 Min (512) RVYRKEE--EELAEELLNYE------I-FIS----YENLEEFKTAKMLYDW---------------INEVPEDEILKTYKVEPGILRYKVEVAKW
      Hel308 Mma (578) RIRPNEE--LDLILEMDKMG------LKDYS----IENIEAFKNSKMFCDW---------------VSEIPEEIILEKYGVEPGILRYKVEQAKW
    Hel308 Mmah (538) LRNRDYEIINDYVMNHTEEFI---EVPSPFKQIEYEWFLSEVKTALLLLEW---------------INEKSLEKIVENYQVGEGDIYASSDIAEW
    Hel308 Mmar (589) LRSGDREEYEMELFEREEELLG--PTPSEFEEGRFEDWLSALKTARLLEDW---------------ATEVDEATITDRYGVGPGDIRGKVETAQW
    Hel308 Mmaz (538) MRSQDYQEVNDYVMAHAGEFS---KVPNPFNIAEYEWFLGEVKTSLLLMDW---------------IHEKPENEICLKFGIGEGDIHATADIAEW
      Hel308 Mok (600) RVRRKEE--NDLINDMIKLDIDVDDVIYGIS----SENLEYFKNAKLFYDW---------------INEIPEEELLLGYNIEPGILRYNVEQAKW
      Hel308 Mth (530) VQQS-DNWLEDFISEHSSELG---NEKN------FDWLLREVKTASMLMDW---------------INEVHEDRIEDRYSISPGDLVRIAETAEW
      Hel308 Mzh (544) MRSSDYENVNMYVLQNKDKFI---SMPSPFKMIEYEWFLGEVKTALLLLDW---------------INEVPADDICKKYGIGEGDIRMFSETAVW
      Hel308 Nma (581) LRSGEDEKFGELYYERERELLG--DAPSEFEEERFEDWLAALKTGKLLEDW---------------ATEDDEEQITERYKIGPGDLRGKVDTAEW
      Hel308 Nth (559) CERKNFEFIDNMAQNHLEEQR---------------------KPFELYNIK---------------PNERVIWNNGDEILFETYTGTKIFQTLAW
      Hel308 Pfu (530) YSKREFERLEEEYYEFKDRLYFDDPYISGYDPYLERKFFRAFKTALVLLAW---------------INEVPEGEIVEKYSVEPGDIYRIVETAEW
      Hel308 Sso (545) VGRNEEEELIELLEDLDCELL----IEEPYEEDEYSLYINALKVALIMKDW---------------MDEVDEDTILSKYNIGSGDLRNMVETMDW
      Hel308 Tba (552) IKRKEQESYLDYAYEMEDYLYRSIPYWEDYE---FQKFLSEVKTAKLLLDW---------------INEVSEAKLIEAYGIDTGDLYRIIELADW
      Hel308 Tga (531) ARRREMEDYLDLAYELEDKLYASIPYYEDSR---FQGFLGQVKTAKVLLDW---------------INEVPEARIYETYSIDPGDLYRLLELADW
      Hel308 Tsi (554) VKRKEQEDLLDYAYEMEEYLYQNIPYWEDYK---FEKFLGETKTAKLLLDW---------------INEVNDVKILETYEIDTGDLYRILELVDW
      Hel308 Mja (531) RVYNSEE--LNLIDEMDSLGIK----------SFEIEDLEAFKTAKMLYDW---------------INEVPEDEILKRYKIEPGILRYKVENAVW
      Consensus (856) LR D E L E I E E F FE FL VKTA LL DW I EV ED I ERYGIGPGDL VE AEW
                                        951 1045
      Hel308 Mbu (640) LMHAAAKLAELLGVEYSS--------HAYSLEKRIRYGSGLDLMELVGIRGVGRVRARKLYNAGFVS---------VAKLKGADISVLSKLVGP-
      Hel308 Afu (600) LSNAMNRIAEEVG-N--T--------SVSGLTERIKHGVKEELLELVRIRHIGRVRARKLYNAGIRN---------AEDIVRHREKVASLIGRG-
      Hel308 Csy (614) LLRCIWEISKHQERPDLLG-------ELDVLRSRVAYGIKAELVPLVSIKGIGRVRSRRLFRGGIKG---------PGDLAAVPVERLSRVEGIG
      Hel308 Dth (848) FNLLKFDYRVLQGLSDYDFSSLPTLDMLREIEARLGHRLSLDHLARHTLGTNKSANGLMALKWWKEGELDKIVEYCRQDVSVTRDLYLFGRDKGY
      Hel308 Fac (584) ISYSLYRLGSMFDKENEN--------NLLHLNIRIKEGVKEEIIRIIEIPQVGRVRGRRLYNNGFKS---------IDDIANARVEDISRIFGFS
      Hel308 Hla (693) LLRAAETLARDVEGVDGDVVV-----AVREARKRIEYGVREELLDLAGVRNVGRKRARRLFEAGIET---------RADLREADKAVVLGALRGR
      Hel308 Hpa (620) LLNAAERLAAELQRDDAEGIPSATTTAVREARKRVEYGVEEELLDLAGVRNVGRKRARRLYEAGIES---------RADLREADKSVVLGALRGR
      Hel308 Htu (651) LLGAAESLAAEIDSEWTV--------AVREARARVEHGVGEELLELVSVGGVGRKRARRLYDAGIEE---------PADLRSADKGIVLSVLKG-
      Hel308 Hvo (696) LLGAAERLATELD---LDSVY-----AVREAKKRVEYGVREELLDLAGVRGVGRKRARRLFEAGVET---------RADLREADKPRVLAALRGR
      Hel308 Mac (615) IMHVATQLARLLDLKGAK--------EAAELEKRIHYGAGPELMDLLDIRGIGRVRARKLYGAGFKS---------TADLAGATPEKVAALVGP-
      Hel308 Mba (614) IMHVTSQLAGLLDLKGAR--------EAAELEKRIHYGAAPELIDLLNIRGIGRVRARKLYEAGFKS---------SAELAEVDPEKVAALLGP-
      Hel308 Mbo (608) LLHATSQLARMFVPKFYG--------QIADCEICMKNGIRRELLPLVRLRGIGRVRARRLFNNGITS---------PEELSRHKKEDLVKILGS-
      Hel308 Mev (615) LMHSAVNLANLTDLDAD---------KAQELEKRIHHGVNKDLIQLVSISNIGRVRARKLYEAGIQS---------VSDIKNTKLHILSNYLGR-
      Hel308 Mfe (601) LMHALKEMAKIIGKN---------SEIPEKLEIRLEYGAKEDIIELLNVKYIGRVRARKLYNAGIRN---------VEDIINNPSK---VASIIG
      Hel308 Mfr (601) LLHASGRLARLVAPEHRD--------AVEETTLRVRHGIRRELIPLVRVKGIGRVRARRLFNNGITG---------PELLAAADPSVVGHIVGG-
      Hel308 Mhu (608) LLHGTERLVSVEMPEMSQ--------VVKTLSVRVHHGVKSELLPLVALRNIGRVRARTLYNAGYPD---------PEAVARAGLSTIARIIGE-
      Hel308 Mig (604) MIYSTKEIAKLLNRN---------IDTLSKLEIRLEYGAKEDIIELLKIKYVGRARARKLYDAGIRS---------VEDIINNPKK---VASLLG
      Hel308 Min (579) LSYSLKEIAKILNKEVP------------NLELRLEYGAKEELLELLKIKYIGRVRARKLYSAGIRN---------REDIIKNPKK---VANILG
      Hel308 Mma (646) MIYSTKEIAKLIHLDNSE-----IYKSLLKMEVRIEYGAKEELIELLNVKNVGRIRSRKLYDAGIRS---------KIEINKNPEK---ILELFG
    Hel308 Mmah (615) LMHATQRIASRINPQLET--------ECAKLEKRIHYGAGSELIELVEIPNVGRARARKLFKKGYRS---------RQKLATADEKQLAGIVGP-
    Hel308 Mmar (667) LLGAAESLASEVDLDAAR--------AISEARIRVEHGVREELVDLAGVRGVGRKRARRLFQAGITD---------RAQLRDADKAVVLAALRGR
    Hel308 Mmaz (615) IMHVTAQLAGLLDLKGAK--------EASELEKRIRYGAAPELMDLLDIRSVGRVRARKLYEAGFKS---------TAELAAASPEHIAVLVGP-
      Hel308 Mok (674) MIHSAKEIFNLLNIDNKV-----IKDCLNDLEIRMEYGAKQDIIELLKIKHIGRARARILYNAGIKN---------ANDIINNQKN---IINLLG
      Hel308 Mth (600) LMSALHRISKHMDLGVTY--------LAERLALRIHYGAGDELLQLLELKGIGRVRARKLYQAGYRS---------LEDLKAADKSTLSEILGP-
      Hel308 Mzh (621) LMHATSRLSGLLKVSEASE-------KSKELEKRLSYGINSELVNIVALKGIGRVRARKIYENGYRS---------IDDLKKADPLKLSKIVGS-
      Hel308 Nma (659) LLGAAESLASEIDSEWAV--------AVREARARVEHGVGEELLELVSVSGIGRKRARRLYAAGIEE---------PAALRSADKGVILHVLKG-
      Hel308 Nth (618) ILRSYNVNIKEIDGIGRIN-----IEGGIDLPGVLQDIKETDWRPEYLLDFTLEQEKFKSKFSPYLP---------KDLQDKMHIAHLVDIEGVK
      Hel308 Pfu (610) LVYSLKEIAKVLG-AYE------IVDYLETLRVRVKYGIREELIPLMQLPLVGRRRARALYNSGFRS---------IEDISQARPEELLKIEGIG
      Hel308 Sso (621) LTYSAYHLSRELKLNEHAD-------KLRILNLRVRDGIKEELLELVQISGVGRKRARLLYNNGIKE---------LGDVVMNPDKVKNLLGQK-
      Hel308 Tba (629) LMYSLIELAKVLNAGGE------TIKYLRRLHLRLKHGVREELLELVELPMIGRRRARALYNAGFKN---------VNDIVKAKPSELLAVEGIG
      Hel308 Tga (608) LMYSLIELYKLFEPKEE------ILNYLRDLHLRLRHGVREELLELVRLPNIGRKRARALYNAGFRS---------VEAIANAKPAELLAVEGIG
      Hel308 Tsi (631) LMYSLIELYKLFDPKPE------VLDFLKKLHIRVKHGVREELLELITLPMIGRKRARALYNAGFKG---------IDDIVRAKASELLKVEGIG
      Hel308 Mja (599) IMHALKEIAKLIGKSSDI---------PEKLEIRLEYGAKEDIIELLSIKYIGRVRARKLYNAGIRS---------IEDIINNPSK---VASIIG
      Consensus (951) LMHA LAKLL L EL IRI YGVKEELLELV IR IGRVRARKLY AGIRS DL A L ILG
                                        1046 1140
      Hel308 Mbu (717) -KVAYNILSGIGVRVNDKHFNSAPISSNTLD--------------------TLLDKNQKTFNDFQ------------------------------
      Hel308 Afu (674) --IAERVVEGISVKSLNPESAAALEHHHHHH----------------------------------------------------------------
      Hel308 Csy (693) ATLANNIKSQLRKGG--------------------------------------------------------------------------------
      Hel308 Dth (943) LLFKNKAGKKVRIPVSWQDTAFQV-----------------------------------------------------------------------
      Hel308 Fac (662) TKLAKDIIENAGKLNNRYYR---------------------------------------------------------------------------
      Hel308 Hla (774) ERTAERILEHAGREDPSMDDVRPDKSASAAATAGS--------------ASDEDGEGQASLGDFR------------------------------
      Hel308 Hpa (706) KKTAENILENVGRQDPSLDDVEADAET---------AA---------TSARATNDGGQQSLGDFE------------------------------
      Hel308 Htu (728) EKTAENILENAGREDPSMDGVEPADGGPAVGAATNGSSGGSETDETGRADAAESDDSQSSLGDF-------------------------------
      Hel308 Hvo (774) RKTAENILEAAGRKDPSMDAVDEDDAPDDAVPDDA---G--------FETAKERADQQASLGDFEGS----------------------------
      Hel308 Mac (692) -KIAERIFRQIGRREAVSEISDSERLEKS------------------------SQDGQSTISDF-------------------------------
      Hel308 Mba (691) -KIADRIFKQIRGRGTSSGIIASEPPEKS------------------------PYSGQKTISDY-------------------------------
      Hel308 Mbo (685) -GIAEQVLEQLHPSKDTGKKEPPSGDKNTN------------------------PG-QSTLFHFG------------------------------
      Hel308 Mev (691) -KTAYKVLEQLGVEPEDNQQIDEEPESIKSY--------------------SGNDQGQKTFNDF-------------------------------
      Hel308 Mfe (675) EKITKKILEDLG--IKFGQ---------------------------------------QKLIF--------------------------------
      Hel308 Mfr (678) -KTAESII---------------------------------------------------------------------------------------
      Hel308 Mhu (685) -GIARQVIDEITGVKRSGIHSSDDDYQQKT------------------------PE-LLTDIPGIGKKMAEKLQNAGIITVSDLLTADEVLLSDV
      Hel308 Mig (678) EKIAKKILGELG--MKFGQ---------------------------------------QTLQI--------------------------------
      Hel308 Min (650) EKISKKIFEELG--VRYGQ---------------------------------------QRLI---------------------------------
      Hel308 Mma (724) EKIGKKILGEHG--MKYGQ---------------------------------------QTLLNFN------------------------------
    Hel308 Mmah (692) -KIAQKILSYLGRETDSNGYVEPETLENK-------------------------KQ-QKTFQDFI------------------------------
    Hel308 Mmar (745) RKTAENVLENAGHRDPSMEGVEPAPDVSVDLNDGADGD---------ASAESTANDDQASLGDF-------------------------------
    Hel308 Mmaz (692) -KITERIFKQIGRREAVSEFSDIEPLEKG------------------------SSDGQRTISDY-------------------------------
      Hel308 Mok (752) EKIARKILSELGVDTKFGQ---------------------------------------MRLSI--------------------------------
      Hel308 Mth (677) -KIAEGVISQLK-EPGVSA----------------------------------------------------------------------------
      Hel308 Mzh (699) -KISQKILKQLDIDVDISEIKEKDSDTVP-E--------------------P--ESSQKTISDFT------------------------------
      Hel308 Nma (736) EKTAENILENAGREEPSMDGVEPIPVEGGSGSGSSNSSGSSEPNADANATEDDADDNQSSLGDF-------------------------------
      Hel308 Nth (699) TFLENKKIKEIKL----------------------------------------------------------------------------------
      Hel308 Pfu (689) VKTVEAIFKFLGKNVKISE-----------------------------------KPRKSTLDYFLKS----------------------------
      Hel308 Sso (699) -LGEKVVQEAARLLNRFH-----------------------------------------------------------------------------
      Hel308 Tba (709) VKVLERIYRHFGVELPLLKNIKDPDKPEDKPKEKP-------------------KPKKGTLDYFLK-----------------------------
      Hel308 Tga (688) AKILDGIYRHLGIEKRVTE-----EK----------------------------PKRKGTLEDFLR-----------------------------
      Hel308 Tsi (711) IGVIEKIYQHFGVELPTNE-----KK--------K-------------------KVKKGTLDEFFK-----------------------------
      Hel308 Mja (673) EKIAKKILDELGVKFGQQKLSFSGGSAWSHPQFEKGGGSGGGSGGSAWSHPQFEK----KL----------------------------------
      Consensus (1046) KIAEKIL LG TL F
                                        1141 1186
      Hel308 Mbu (761) ----------------------------------------------
      Hel308 Afu (703) ----------------------------------------------
      Hel308 Csy (708) ----------------------------------------------
      Hel308 Dth (967) ----------------------------------------------
      Hel308 Fac (682) ----------------------------------------------
      Hel308 Hla (825) ----------------------------------------------
      Hel308 Hpa (753) ----------------------------------------------
      Hel308 Htu (792) ----------------------------------------------
      Hel308 Hvo (830) ----------------------------------------------
      Hel308 Mac (731) ----------------------------------------------
      Hel308 Mba (730) ----------------------------------------------
      Hel308 Mbo (724) ----------------------------------------------
      Hel308 Mev (734) ----------------------------------------------
      Hel308 Mfe (697) ----------------------------------------------
      Hel308 Mfr (685) ----------------------------------------------
      Hel308 Mhu (754) LGAARARKVLAFLSNSEKENSSSDKTEEIPDTQKIRGQSSWEDFGC
      Hel308 Mig (700) ----------------------------------------------
      Hel308 Min (671) ----------------------------------------------
      Hel308 Mma (748) ----------------------------------------------
    Hel308 Mmah (730) ----------------------------------------------
    Hel308 Mmar (800) ----------------------------------------------
    Hel308 Mmaz (731) ----------------------------------------------
      Hel308 Mok (776) ----------------------------------------------
      Hel308 Mth (694) ----------------------------------------------
      Hel308 Mzh (740) ----------------------------------------------
      Hel308 Nma (800) ----------------------------------------------
      Hel308 Nth (712) ----------------------------------------------
      Hel308 Pfu (721) ----------------------------------------------
      Hel308 Sso (716) ----------------------------------------------
      Hel308 Tba (756) ----------------------------------------------
      Hel308 Tga (721) ----------------------------------------------
      Hel308 Tsi (745) ----------------------------------------------
      Hel308 Mja (730) ----------------------------------------------
      Consensus (1141) ----------------------------------------------
The number below the * indicates the SEQ ID NO. The “-” are shown for alignment purposes only and do not form part of the sequences.
```

1-43. (canceled)

44. A method of controlling the movement of a polynucleotide through a transmembrane pore, comprising:
(a) contacting the polynucleotide with a transmembrane pore under an applied voltage field such that a portion of the polynucleotide is captured by the transmembrane pore, wherein the pore is present in a membrane;
(b) contacting the polynucleotide with a Hel308 helicase on the cis side of the membrane such that the helicase binds to the polynucleotide, thereby controlling the movement of the polynucleotide through the transmembrane pore against the applied voltage field from the trans side of the membrane to the cis side of the membrane; and
(c) measuring an ionic current as the polynucleotide moves through the transmembrane pore.

45. The method of claim 44, wherein the polynucleotide is modified by methylation, by oxidation, by damage, with one or more proteins, or with one or more labels, tags, or spacers.

46. The method of claim 44, wherein the transmembrane pore is a protein pore.

47. The method of claim 46, wherein the protein pore is selected from the group consisting of: α-hemolysin, leukocidin, Mycobacterium smegmatis porin A (MspA), outer membrane porin F (OmpF), outer membrane porin G (OmpG), outer membrane phospholipase A, Neisseria autotransporter lipoprotein (NalP) and WZA.

48. The method of claim 44, wherein the Hel308 helicase comprises the amino acid motif Q-X₁-X₂-G-R-A-G-R (SEQ ID NO: 8), wherein X₁ is C, M or L and X₂ is A, F, M, C, V, L, I, S, T or P.

49. A method of controlling the movement of a polynucleotide through a transmembrane pore, comprising:
(a) providing in an aqueous solution the transmembrane pore and a membrane, wherein the transmembrane pore is present in the membrane, and wherein the aqueous solution comprises a salt at a concentration in a range of 0.3 M to 3 M;
(b) combining, in the aqueous solution of step (a), the polynucleotide and a Hel308 helicase, wherein the helicase binds to the polynucleotide to form a polynucleotide-helicase complex, thereby controlling the movement of the polynucleotide through the transmembrane pore; and
(c) measuring, during an application of a potential across the transmembrane pore, an ionic current as the polynucleotide moves through the transmembrane pore.

50. The method of claim 49, wherein the polynucleotide is modified by methylation, by oxidation, by damage, with one or more proteins or with one or more labels, tags or spacers.

51. The method of claim 49, wherein the transmembrane pore is a protein pore.

52. The method of claim 51, wherein the transmembrane protein pore is selected from the group consisting of: α-hemolysin, leukocidin, Mycobacterium smegmatis porin A (MspA), outer membrane porin F (OmpF), outer membrane porin G (OmpG), outer membrane phospholipase A, Neisseria autotransporter lipoprotein (NalP) and WZA.

53. The method of claim 49, wherein the Hel308 helicase comprises the amino acid motif Q-X₁-X₂-G-R-A-G-R (SEQ ID NO: 8), wherein X₁ is C, M or L and X₂ is A, F, M, C, V, L, I, S, T or P.

54. The method of claim 49, wherein the salt is KCl.

55. The method of claim 54, wherein the salt concentration is at least 1.0 M.

56. A method of controlling the movement of a polynucleotide through a transmembrane pore, comprising:
(a) contacting a polynucleotide with a transmembrane pore, wherein a first helicase is bound to the polynucleotide such that the first helicase controls the movement of the polynucleotide through the pore;
(b) further contacting the polynucleotide with a second helicase as the first helicase controls the movement of the polynucleotide through the transmembrane pore, wherein the second helicase binds to an internal polynucleotide of the polynucleotide at a location on the polynucleotide different than where the first helicase is bound without concurrent binding to a terminal nucleotide of the polynucleotide, and wherein the second helicase controls movement of the polynucleotide through the pore after the first helicase disengages from the polynucleotide; and
(c) measuring an ionic current as the polynucleotide moves through the transmembrane pore.

57. The method of claim 56, wherein the one or more interactions are measured along a length of the polynucleotide that comprises at least 10 nucleotides.

58. The method of claim 56, wherein the one or more interactions are measured along a length of the polynucleotide that comprises 10-100 nucleotides.

59. The method of claim 56, wherein the second helicase is a Hel308 helicase, Hel308 Tga, Hel308 Mhu or Hel308 Csy.

60. The method of claim 56, wherein the polynucleotide is modified by methylation, by oxidation, by damage, with one or more proteins or with one or more labels, tags or spacers.

61. The method of claim 56, wherein the transmembrane pore is a protein pore.

62. The method of claim 61, wherein the transmembrane protein pore is selected from the group consisting of: α-hemolysin, leukocidin, Mycobacterium smegmatis porin A (MspA), outer membrane porin F (OmpF), outer membrane porin G (OmpG), outer membrane phospholipase A, Neisseria autotransporter lipoprotein (NalP) and WZA.

63. The method of claim 56, wherein the method further comprises the step of applying a voltage across the pore to form a complex between the pore and the first helicase; and wherein at least a portion of the polynucleotide is double stranded.


SEQUENCE LISTING (78 sequences)

SEQ ID NO: 1 - DNA, 558 bp, synthetic construct
  misc_feature 1..558: note = Synthetic Polynucleotide
  source 1..558: mol_type = other DNA
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 2 - protein, 184 aa, synthetic construct
  REGION 1..184: note = Synthetic Polypeptide
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 3 - DNA, 885 bp, synthetic construct
  misc_feature 1..885: note = Synthetic Polynucleotide
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, synthetic construct
  REGION 1..293: note = Synthetic Polypeptide
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, Mycobacterium smegmatis
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - protein, 8 aa, synthetic construct
  REGION 1..8: note = Synthetic Polypeptide
  VARIANT 2: note = May be C, M or L
QXXGRAGR

SEQ ID NO: 9 - protein, 9 aa, synthetic construct
  REGION 1..9: note = Synthetic Polypeptide
  VARIANT 2: note = May be C, M or L
QXXGRAGRP

SEQ ID NO: 10 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 11 - protein, 8 aa, synthetic construct
  REGION 1..8: note = Synthetic Polypeptide
QMAGRAGR

SEQ ID NO: 12 - protein, 9 aa, synthetic construct
  REGION 1..9: note = Synthetic Polypeptide
QMAGRAGRP

SEQ ID NO: 13 - protein, 720 aa, Pyrococcus furiosus
MRVDELRVDERIKSTLKERGIESFYPPQAEALKSGILEGKNALISIPTASGKTLIAEIAMVHRILTQGGKAVYIVPLKALAEEKFQEFQDWEKIGLRVAMATGDYDSKDEWLGKYDIIIATAEKFDSLLRHGSSWIKDVKILVADEIHLIGSRDRGATLEVILAHMLGKAQIIGLSATIGNPEELAEWLNAELIVSDWRPVKLRRGVFYQGFVTWEDGSIDRFSSWEELVYDAIRKKKGALIFVNMRRKAERVALELSKKVKSLLTKPEIRALNELADSLEENPTNEKLAKAIRGGVAFHHAGLGRDERVLVEENFRKGIIKAVVATPTLSAGINTPAFRVIIRDIWRYSDFGMERIPIIEVHQMLGRAGRPKYDEVGEGIIVSTSDDPREVMNHYIFGKPEKLFSQLSNESNLRSQVLALIATFGYSTVEEILKFISNTFYAYQRKDTYSLEEKIRNILYFLLENEFIEISLEDKIRPLSLGIRTAKLYIDPYTAKMFKDKMEEVVKDPNPIGIFHLISLTPDITPFNYSKREFERLEEEYYEFKDRLYFDDPYISGYDPYLERKFFRAFKTALVLLAWINEVPEGEIVEKYSVEPGDIYRIVETAEWLVYSLKEIAKVLGAYEIVDYLETLRVRVKYGIREELIPLMQLPLVGRRRARALYNSGFRSIEDISQARPEELLKIEGIGVKTVEAIFKFLGKNVKISEKPRKSTLDYFLKS

SEQ ID NO: 14 - protein, 8 aa, synthetic construct
  REGION 1..8: note = Synthetic Polypeptide
QMLGRAGR

SEQ ID NO: 15 - protein, 9 aa, synthetic construct
  REGION 1..9: note = Synthetic Polypeptide
QMLGRAGRP

SEQ ID NO: 16 - protein, 829 aa, Haloferax volcanii
MRTADLTGLPTGIPEALRDEGIEELYPPQAEAVEAGLTDGESLVAAVPTASGKTLIAELAMLSSVARGGKALYIVPLRALASEKKAEFERWEEYGIDVGVSTGNYESDGEWLSSRDIIVATSEKVDSLVRNNAAWMDQLTCVVADEVHLVDDRHRGPTLEVTLAKLRRLNTNLQVVALSATVGNAGVVSDWLDAELVKSDWRPIDLKMGVHYGNAVSFADGSQREVPVGRGERQTPALVADALEGDGEGDQGSSLVFVNSRRNAESAARRMADVTERYVTGDERSDLAELAAEIRDVSDTETSDDLANAVAKGAAFHHAGLAAEHRTLVEDAFRDRLIKCICATPTLAAGVNTPSRRVVVRDWQRYDGDYGGMKPLDVLEVHQMMGRAGRPGLDPYGEAVLLAKDADARDELFERYIWADAEDVRSKLAAEPALRTHLLATVASGFAHTREGLLEFLDQTLYATQTDDPERLGQVTDRVLDYLEVNGFVEFEGETIQATPVGHTVSRLYLDPMSAAEIIDGLEWAADHRTEKLRALAGETPEKPTRDRSESDESGGFQRASEMVADDGDGGGGEDGVGANGDGDSDDADGVETDRTYPTPLGLYHLVCRTPDMYQLYLKSGDRETYTELCYEREPEFLGRVPSEYEDVAFEDWLSALKTAKLLEDWVGEVDEDRITERYGVGPGDIRGKVETSEWLLGAAERLATELDLDSVYAVREAKKRVEYGVREELLDLAGVRGVGRKRARRLFEAGVETRADLREADKPRVLAALRGRRKTAENILEAAGRKDPSMDAVDEDDAPDDAVPDDAGFETAKERADQQASLGDFEGS

SEQ ID NO: 17 - protein, 8 aa, synthetic construct
  REGION 1..8: note = Synthetic Polypeptide
QMMGRAGR

SEQ ID NO: 18 - protein, 9 aa, synthetic construct
  REGION 1..9: note = Synthetic Polypeptide
QMMGRAGRP

SEQ ID NO: 19 - protein, 824 aa, Halorubrum lacusprofundi
MQPSSLSGLPAGVGEALEAEGVAELYPPQEAAVEAGVADGESLVAAVPTASGKTLIAELAMLSSIERGGKALYIVPLRALASEKKTEFERWEEFGVTVGVSTGNYESDGEWLATRDIIVATSEKVDSLIRNGAPWIDDLTCVVSDEVHLVDDPNRGPTLEVTLAKLRKVNPGLQTVALSATVGNADVIAEWLDAELVESDWRPIDLRMGVHFGNAIDFADGSKREVPVERGEDQTARLVADALDTEEDGQGGSSLVFVNSRRNAESSARKLTDVTGPRLTDDERDQLRELADEIRSGSDTDTASDLADAVEQGSAFHHAGLRSEDRARVEDAFRDRLIKCISATPTLAAGVNTPARRVIVRDWRRYDGEFGGMKPLDVLEVHQMCGRAGRPGLDPYGEAVLLANDADTKEELFERYLWADPEPVRSKLAAEPALRTHVLATVASGFASTRDGLLSFLDNTLYATQTDDEGRLAAVTDTVLDYLAVNDFIERDRDGGSESLTATGIGHTVSRLYLDPMSAAEMIDGLRSVARDAADTGASAEADNGEFVRTGDADDASGGDEPGFGTYTRAGDDESGERETENEETDEEETEASEVTPLGLYHLISRTPDMYELYLKSGDRETYTELCYERETEFLGDVPSEYEDVRFEDWLASLKTARLLEDWVNEVDEDRITERYGVGPGDIRGKVDTAEWLLRAAETLARDVEGVDGDVVVAVREARKRIEYGVREELLDLAGVRNVGRKRARRLFEAGIETRADLREADKAVVLGALRGRERTAERILEHAGREDPSMDDVRPDKSASAAATAGSASDEDGEGQASLGDFR

SEQ ID NO: 20 - protein, 8 aa, synthetic construct
  REGION 1..8: note = Synthetic Polypeptide
QMCGRAGR

SEQ ID NO: 21 - protein, 9 aa, synthetic construct
  REGION 1..9: note = Synthetic Polypeptide
QMCGRAGRP

SEQ ID NO: 22 - protein, 707 aa, unidentified
  source 1..707: note = Cenarchaeum symbiosum
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 23 - protein, 8 aa, synthetic construct
  REGION 1..8: note = Synthetic Polypeptide
QLCGRAGR

SEQ ID NO: 24 - protein, 9 aa, synthetic construct
  REGION 1..9: note = Synthetic Polypeptide
QLCGRAGRP

SEQ ID NO: 25 - protein, 715 aa, Sulfolobus solfataricus
MSLELEWMPIEDLKLPSNVIEIIKKRGIKKLNPPQTEAVKKGLLEGNRLLLTSPTGSGKTLIAEMGIISFLLKNGGKAIYVTPLRALTNEKYLTFKDWELIGFKVAMTSGDYDTDDAWLKNYDIIITTYEKLDSLWRHRPEWLNEVNYFVLDELHYLNDPERGPVVESVTIRAKRRNLLALSATISNYKQIAKWLGAEPVATNWRPVPLIEGVIYPERKKKEYNVIFKDNTTKKVHGDDAIIAYTLDSLSKNGQVLVFRNSRKMAESTALKIANYMNFVSLDENALSEILKQLDDIEEGGSDEKELLKSLISKGVAYHHAGLSKALRDLIEEGFRQRKIKVIVATPTLAAGVNLPARTVIIGDIYRFNKKIAGYYDEIPIMEYKQMSGRAGRPGFDQIGESIVVVRDKEDVDRVFKKYVLSDVEPIESKLGSERAFYTFLLGILSAEGNLSEKQLENFAYESLLAKQLVDVYFDRAIRWLLEHSFIKEEGNTFALTNFGKRVADLYINPFTADIIRKGLEGHKASCELAYLHLLAFTPDGPLVSVGRNEEEELIELLEDLDCELLIEEPYEEDEYSLYINALKVALIMKDWMDEVDEDTILSKYNIGSGDLRNMVETMDWLTYSAYHLSRELKLNEHADKLRILNLRVRDGIKEELLELVQISGVGRKRARLLYNNGIKELGDVVMNPDKVKNLLGQKLGEKVVQEAARLLNRFH

SEQ ID NO: 26 - protein, 8 aa, synthetic construct
  REGION 1..8: note = Synthetic Polypeptide
QMSGRAGR

SEQ ID NO: 27 - protein, 9 aa, synthetic construct
  REGION 1..9: note = Synthetic Polypeptide
QMSGRAGRP

SEQ ID NO: 28 - protein, 685 aa, Methanogenium frigidum
MDLSLPKAFIQYYKDKGIESLYPPQSECIENGLLDGADLLVAIPTASGKTLIAEMAMHAAIARGGMCLYIVPLKALATEKAQEFKGKGAEIGVATGDYDQKEKRLGSNDIVIATSEKVDSLLRNGVPWLSQVTCLVVDEVHLIDDESRGPTLEMVITKLRHASPDMQVIGLSATIGNPKELAGWLGADLITSDWRPVDLREGICYHNTIYFDNEDKEIPAPAKTEDINLLLDCVADGGQCLVFVSSRRNAEGYAKRAATALKCSHAALDSIAEKLEAAAETDMGRVLATCVKKGAAFHHAGMNRMQRTLVEGGFRDGFIKSISSTPTLAAGLNLPARRVIIRDYLRYSGGEGMRPIPVREYRQMAGRAGRPHLDPYGEAILIAKTEYAVNDLHEEYVEAPDEDVTSRCGEKGVLTAHILSLIATGYARSYDELMAFLEKTLYAYQHTGKKALTRTLDDALGFLTEAEMVTDLSGMLHATEYGDLTSRLYIDPHSAEIITTALREEGELTDLALLQLLCMTPDMFTLYVKKNDLGTLEKFFFEHEEEFRTEFSYDEMEDFFRSLKTAMLLSDWTDEIGDDTICTRFGVGPGDIFNAVQGISWLLHASGRLARLVAPEHRDAVEETTLRVRHGIRRELIPLVRVKGIGRVRARRLFNNGITGPELLAAADPSVVGHIVGGKTAESII

SEQ ID NO: 29 - protein, 775 aa, Methanothermococcus okinawensis
MLMLMEVLKENGIAELRPPQKKVVEGGLLNKNKNFLICIPTASGKTLIGEMAFINHLLDNNKTPTNKKGLFIVPLKALANEKYEEFKGKYEKYGLKIALSIGDFDEKEDLKGYDLIITTAEKLDSLIRHKVEWIKDISVVVIDEIHLIGDESRGGTLEVLLTKLKTKKTIQIIGLSATIGNPEELAKWLNAELIVDEWRPVKLKKGIGYGNKIMFIDDNGNTINEVIVDEISKNNMFNLVVDSILKDGSCIIFCNSKRGAVGEAKKLNLKKYLSPDEISELRHLKEEVLSVLDNPTKTCKDLAECIEKGVAFHHAGLTYEQRKIVEEGFRKKLIKAICCTPTLSAGINMPCRRAIIRDLKRFSSRGYIPIPKMEIHQCIGRAGRPNLDPYGEGIIYINNTENPELIENAKNYLIGNVEEIYSKLSNQKVLRTHMLGLITTGDIKNKNDLEEFIKNTFYAYQYQNTKKILENIYEITNFLEKNGFIELNYRRDENKDKSNNSHNNKKNISNTNNSIKMLVLDNNNSLTIKSRHEEDVYYNITPLGKKVSELYIDPLSAEYIIDGLKNLHKKTLSNPKNMECYILHILYIISKTTEMQPVLRVRRKEENDLINDMIKLDIDVDDVIYGISSENLEYFKNAKLFYDWINEIPEEELLLGYNIEPGILRYNVEQAKWMIHSAKEIFNLLNIDNKVIKDCLNDLEIRMEYGAKQDIIELLKIKHIGRARARILYNAGIKNANDIINNQKNIINLLGEKIARKILSELGVDTKFGQMRLSI

SEQ ID NO: 30 - protein, 8 aa, synthetic construct
  REGION 1..8: note = Synthetic Polypeptide
QCIGRAGR

SEQ ID NO: 31 - protein, 9 aa, synthetic construct
  REGION 1..9: note = Synthetic Polypeptide
QCIGRAGRP

SEQ ID NO: 32 - protein, 699 aa, Methanotorris igneus
MQKYSHVFEVLKENGIKELRPPQKKVIEKGLLNKEKNFLICIPTASGKTLIGEMALINHLLDENKTPTNKKGLFIVPLKALASEKYEEFKRKYEKYGLKVALSIGDYDEKEDLSSYNIIITTAEKLDSLMRHEIDWLNYVSVAIVDEIHMINDEKRGGTLEVLLTKLKNLDVQIIGLSATIGNPEELAEWLNAELIIDNWRPVKLRKGIFFQNKIMYLNGACKELPNFSNNPMLNLVLDCVKEGGCCLVFCNSKNGAVSEAKKLNLKKYLSNSEKYELQKLKEEILSILDPPTETCKTLAECLEKGVAFHHAGLTYEHRKIVEEGFRNKLIKVICCTPTLSAGINIPCRRAIVRDLMRFSNGRMKPIPIMEIHQCIGRAGRPGLDPYGEGIIFVKNERDLERAEQYLEGKPEYIYSKLSNQAVLRTQLLGMIATREIENEFDLISFIKNTFYAHQYGNLGGVLRNIKEVINFLEENDFIADYFPTKLGKRVSELYIDPLSAKIIIDGLKEMGNVDNEELYYLYLISKTLEMMPLLRVNSFEELDLILEMEEAGIYDRTYDDLAAFKNAKMLYDWINEVPEDEILKKYKIEPGILRYKVEQAKWMIYSTKEIAKLLNRNIDTLSKLEIRLEYGAKEDIIELLKIKYVGRARARKLYDAGIRSVEDIINNPKKVASLLGEKIAKKILGELGMKFGQQTLQI

SEQ ID NO: 33 - protein, 720 aa, Thermococcus gammatolerans
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 34 - protein, 755 aa, Thermococcus barophilus
MLSTKPKAYKRFSPIGYAMQVDELSKFGVDERIIRKIKERGISEFYPPQAEALRSGVLNGENLLLAIPTASGKTLVAEIVMLHKLFTGGGKAVYLVPLKALAEEKYREFKTWEDLGVRVAVTTGDYDSSEEWLGKYDIIIATSEKFDSLLRHKSRWIRDVTLIVADEIHLLGSYDRGATLEMILSHMLGKAQILGLSATVGNAEELAEWLNAKLVVSDWRPVKLRKGVFAHGQLIWEDGKVDKFPPQWDSLVIDAVKKGKQALVFVNTRRSAEKEAGMLGKKVRRLLTKPEARRLKELAESLESNPTNDKLKEVLVNGAAFHHAGLGRAERTLIEDAFREGLIKVLTATPTLAMGVNLPSFRVIIRDTKRYSTFGWSDIPVLEIQQMIGRAGRPKYDKEGEAIIVAKTEKPEELMEKYIFGKPEKLFSMLSNDAAFRSQVLALITNFGVESFRELIGFLEKTFYYHQRKDLEILEGKAKSIVYFLLENEFIDIDLNDSFIALPFGIRTSQLYLDPLTAKKFKDALPQIEENPNPLGIFQLLASTPDMGTLSIKRKEQESYLDYAYEMEDYLYRSIPYWEDYEFQKFLSEVKTAKLLLDWINEVSEAKLIEAYGIDTGDLYRIIELADWLMYSLIELAKVLNAGGETIKYLRRLHLRLKHGVREELLELVELPMIGRRRARALYNAGFKNVNDIVKAKPSELLAVEGIGVKVLERIYRHFGVELPLLKNIKDPDKPEDKPKEKPKPKKGTLDYFLK

SEQ ID NO: 35 - protein, 8 aa, synthetic construct
  REGION 1..8: note = Synthetic Polypeptide
QMIGRAGR

SEQ ID NO: 36 - protein, 9 aa, synthetic construct
  REGION 1..9: note = Synthetic Polypeptide
QMIGRAGRP

SEQ ID NO: 37 - protein, 744 aa, Thermococcus sibiricus
MKLNKLKSYINAFLLGMVMSMKVDELKSLGVDERILRLLRERGIEELYPPQADALKTEVLKGKNLVLAIPTASGKTLVAEIVMINKILREGGKTVYLVPLKALAEEKYKEFKFWEKLGIRIAMTTGDYDSTEEWLGKYDIIIATSEKFDSLLRHKSPWIKDINLVIADEIHLLGSYDRGATLEMILAHLDDKAQILGLSATVGNAEEVAEWLNADLVMSEWRPVALRKGVFYHGELFWEDGSIERFPTQWDSLVIDALKKGKQALVFVNTRRSAEKEALLLAGKIQRFLTKPEERKLKQLADGLDTTPTNQKLKEALTKGVAFHHAGLGRTERSIIEDAFREGLIKVITATPTLSAGVNLPAYRVIIRDTKRYSNFGWVDIPVLEIQQMMGRAGRPKYDIEGQAIIIAKTEKPEDLMKRYVLGKPEKLFSMLSNEASFRSQVLALITNFGVGNFKELVNFLERTFYYHQRKNLEALEGKAKSIVYFLFENEFIDIDLNDQFMPLPLGIRTSQLYLDPVTAKKFKDAFEKLEKNPNPLGIFQLLASTPDMSSLRVKRKEQEDLLDYAYEMEEYLYQNIPYWEDYKFEKFLGETKTAKLLLDWINEVNDVKILETYEIDTGDLYRILELVDWLMYSLIELYKLFDPKPEVLDFLKKLHIRVKHGVREELLELITLPMIGRKRARALYNAGFKGIDDIVRAKASELLKVEGIGIGVIEKIYQHFGVELPTNEKKKKVKKGTLDEFFK

SEQ ID NO: 38 - protein, 729 aa, unidentified
  source 1..729: note = Methanosarcina barkeri fusaro
MKIESLDLPDEVKQFYLNSGIMELYPPQAEAVEKGLLEGRNLLAAIPTASGKTLLAELAMLKSILAGGKALYIVPLRALASEKFRRFREFSELGIRVGISTGDYDLRDEGLGVNDIIVATSEKTDSLLRNETVWMQEISVVVADEVHLIDSPDRGPTLEVTLAKLRKMNPSCQILALSATVGNADELAVWLEAELVVSEWRPTELLEGVFFNGTFYCKDREKTVEQSTKDEAVNLALDTLKKDGQCLVFESSRKNCMAFAKKAASTVKKTLSAEDRNALAGIADEILENSETDTSTNLAVCIRSGTAFHHAGLTTPLRELVEDGFRAGRIKLISSTPTLAAGLNLPARRVIIRNYRRYSSEDGMQPIPVLEYKQMAGRAGRPRLDPYGEAVLVAKSYKEFVFLFENYIEANAEDIWSKLGTENALRTHVLSTISNGFARTYDELMDFLEATFFAFQYSNFGLSTVVNECLNFLRQEGMLEKDDALIPTSFGKLVSRLYIDPLSAARIAKGLKGAKSLSELTLLHLVCSTPDMRLLYMRSHDYQDINDYVMAHASEFVKVPSPFDTTEYEWFLGEVKTSLLLLDWIHEKSENEICLKFGTGEGDIHSIADIAEWIMHVTSQLAGLLDLKGAREAAELEKRIHYGAAPELIDLLNIRGIGRVRARKLYEAGFKSSAELAEVDPEKVAALLGPKIADRIFKQIRGRGTSSGIIASEPPEKSPYSGQKTISDY

SEQ ID NO: 39 - protein, 730 aa, Methanosarcina acetivorans
MKIESLDLPDEVKRFYENSGIPELYPPQAEAVEKGLLEGKNLLAAIPTASGKTLLAELAMLKSVLAGGKALYIVPLRALASEKFRRFQDFSELGIRVGISTGDYDRRDEGLGINDIIVATSEKTDSLLRNETAWMQEISVVVVDEVHLIDSADRGPTLEVTLAKLRKMNPFCQILALSATVGNADELAAWLDAELVLSEWRPTDLMEGVFFDGTFFCKDKEKLIEQPTKDEAINLVLDTLREGGQCLVFESSRKNCMGFAKKATSAVKKTLSAEDKEKLAGIADEILENSETDTASVLASCVRAGTAFHHAGLTSPLRELVETGFREGYVKLISSTPTLAAGLNLPARRVIIRSYRRYSSDSGMQPIPVLEYKQMAGRAGRPRLDPYGEAVLLAKSYEELLFLFEKYIEAGAEDIWSKLGTENALRTHVLSTISNGFARTKEELMDFLEATFFAYQYSNFGLSVVVDECLNFLRQEGMLEQDSDALISTMFGKLVSRLYIDPLSAALIAKGLREAGTLTELTLLHLVCSTPDMRLMYMRSQDYQDINDFVMAHAEEFSKVPSPFNIVEYEWFLSEVKTSLLLMDWIHEKPENEICLKFGTGEGDIHTTADIAEWIMHVATQLARLLDLKGAKEAAELEKRIHYGAGPELMDLLDIRGIGRVRARKLYGAGFKSTADLAGATPEKVAALVGPKIAERIFRQIGRREAVSEISDSERLEKSSQDGQSTISDF

SEQ ID NO: 40 - protein, 729 aa, Methanohalophilus mahii
MKIEELDLPSEAIEVYLQAGIEELYPPQADAVEKGLLQGENLLAAIPTASGKTLLAEMAMLKAIKKGGKALYIVPLRALASEKFRDFKRFESLGIKTAISTGDFDSRDEWLGSNDIIVATSEKTDSLLRNSTPWMKDITAVIVDEVHLLDSANRGPTLEVTLAKLKRLNPGAQVVALSATVGNAMEIAQWLEAKLVLSEWRPTYLHEGIFYGDAINFDEDQTFIERRHKEDSVNLVIDTVIQGGQCLVFDSSRRNCVGFAKKCAPAVGELLDRQNRNELEEVAKEVLENGETKLTETLAYCIKKGVAFHHAGLNSAHRRIVEDAFRNNLIKMICSTPTLAAGLNLPARRVIIRSYKRYDPNAGMQPIPVLDYKQMAGRAGRPHLDPYGEAVVIVKTYEEFTDVLERYISASAEDIWSKLGTENALRTHILSTIASGFANCHREILTFLGSTFFAHQQQSWNFEELLEDCLIFLKNEGMLEQDNETIRATELGKMISSLYIDPLSASKIIRGLEKTTHVTDMTLLQLICSTPDMRLLYLRNRDYEIINDYVMNHTEEFIEVPSPFKQIEYEWFLSEVKTALLLLEWINEKSLEKIVENYQVGEGDIYASSDIAEWLMHATQRIASRINPQLETECAKLEKRIHYGAGSELIELVEIPNVGRARARKLFKKGYRSRQKLATADEKQLAGIVGPKIAQKILSYLGRETDSNGYVEPETLENKKQQKTFQDFI

SEQ ID NO: 41 - protein, 730 aa, Methanosarcina mazei
MKIESLDLPDEIKRFYENSGILELYPPQAEAVEKGLLEGKNLLAAIPTASGKTLLAELAMLKSVLNGGKALYIVPLRALASEKFRRFQEFSVLGMRVGISTGDYDRRDEGLGINDIIVATSEKTDSLLRNETAWMQEISVVVADEVHLIDSPDRGPTLEITLSKLRRMNPSCQVLALSATVGNADELAAWLDAELVLSEWRPTDLMEGVFYNGIFYCKDKEKPVGQPTKDEAVNLVLDTIKEGGQCLVFESSRKNCMGFAKKAVSAVKKTLSNEDRETLAGIADEIIENSETDVSSVLATCVRSGTAFHHAGLTTPLRELVENGFREGRIKIISSTPTLAAGLNLPARRVIIRSYRRYSSDSGMQPIPVLEYKQMAGRAGRPRLDPYGEAVLLAKSYEEFVFLFEKYIEAGAEDIWSKLGTENALRTHILSTISNGFARTREELMDFLEATFFAFQYSNFGLSAVVDECLDFLRREGMLEKDPDALVSTVFGKLVSRLYIDPLSAALIAKGLREAGTLTELTLLHLICSTPDMRLMYMRSQDYQEVNDYVMAHAGEFSKVPNPFNIAEYEWFLGEVKTSLLLMDWIHEKPENEICLKFGIGEGDIHATADIAEWIMHVTAQLAGLLDLKGAKEASELEKRIRYGAAPELMDLLDIRSVGRVRARKLYEAGFKSTAELAAASPEHIAVLVGPKITERIFKQIGRREAVSEFSDIEPLEKGSSDGQRTISDY

SEQ ID NO: 42 - protein, 693 aa, unidentified
  source 1..693: note = Methanosaeta thermophila
MLTIRDLIRWLPESVIELYEALGIDELYPPQAEAIERGLLDGRNMIISVPTAAGKTLLAELAMLRGALSGKRSLYIVPLRALASEKFESFSRFSKLGLRVGISTGDFEKRDERLGRNDIIIATSEKADSLIRNGASWVRRIGVLVVDEIHLLDSANRGPTLEMTMTKLMHLNPEMQVIGLSATIANGREIADWIKGEIVSSDWRPVRLREGVLLEDRLVFPDGEIQLENRNRDPVLNLVLDTVDQGGQMLIFESTRRNAESMAKKVSGALQESGETIELAERLSGEGKTAKKLAMCLRHGAAFHHAGLLPEQRRLIELGFRQNVVKVIACTPTLAAGLNLPARRVLIRSYKRYEAGLGTRPIPVMEYRQMAGRAGRPGLDPYGESLIMARSESELQKLMDHYVMGEPEDIWSKLASERALRTHVLATIASRFADSVDSLSRLMASTFYARQQDPSYLGETIASVLEFLVRSDMIDKDLTPTPLGALVSRLYIDPLSAMVMIQEIRGIRRPTVLTLLHVITMTPDMELLFVQQSDNWLEDFISEHSSELGNEKNFDWLLREVKTASMLMDWINEVHEDRIEDRYSISPGDLVRIAETAEWLMSALHRISKHMDLGVTYLAERLALRIHYGAGDELLQLLELKGIGRVRARKLYQAGYRSLEDLKAADKSTLSEILGPKIAEGVISQLKEPGVSA

SEQ ID NO: 43 - protein, 739 aa, Methanosalsum zhilinae
MNINNLNLPEKVKKYYTDTGIVDLYPPQREAVDKGLLDGENIVAAIPTASGKTLLAELCMLKSIGMGGKCLYIVPLKALASEKYSRFREFESLGIKVGIATGDLDSREEWLGKNDIIIATSEKVDSLLRNESSWMKEINTVVADEVHLLNSVNRGPTLEITLAKLIHLNPGSQIIALSATIGNPEDIAGWLGARLVVSEWRPTDLYEGILLDGLLHIGNIKKDIQDESRDDAVNLVIDTVKDKGQCLVFESSRRNCMGFAKKAGKWVSKILDEHDTIQLKSLSQEIGEAGETEIADVLSRCVRQGVAFHHAGLNSEHRRMVEEGFRKNLIKMISSTPTLAAGLNLPARRVIIRSYKRYDPNFGMKPIPVLEYKQMAGRAGRPHLDPYGESVLIARSYDEFMDIMENYVNADPEDIWSKLGTENALRTHVLSTIVNGFAYTYRGLMDFVKMTFFAYQKEASDLHDVIEECVRFLIDNEMIISDSNDILPESAFRSTATGKLISMLYIDPLSGSLIMDGIRKADYFEDITMMHLICSTPDMKNLYMRSSDYENVNMYVLQNKDKFISMPSPFKMIEYEWFLGEVKTALLLLDWINEVPADDICKKYGIGEGDIRMFSETAVWLMHATSRLSGLLKVSEASEKSKELEKRLSYGINSELVNIVALKGIGRVRARKIYENGYRSIDDLKKADPLKLSKIVGSKISQKILKQLDIDVDISEIKEKDSDTVPEPESSQKTISDFT

SEQ ID NO: 44 - protein, 733 aa, Methanohalobium evestigatum
METGKLELPEYVIQFYLDTGIEKLYPPQAEAVEKGLLDNKNLLAAIPTASGKTLISELAMLKSISNGGKCLYIVPLRALASEKFERFKQFSSIGVNIGISTGDFDSTDEWLGSNDIIVATSEKADSLLRNETSWMKDITTIVVDEIHLLDSADRGPTLEITIAKLLRLNPNSQIIGLSATIGNAEEIAGWLDAELVQSQWRPIELYEGVFLEDNINFKQSQKPIKNIVKDTAVNLVLDTIDENGQCLVFESSRRNCAGFAKKAKSKVGKSLDKGLLAELNNIAEEVLETSDTETTKELASCIKRGTAFHHAGLNSAQRKIVEDNFRNNKIKVISSTPTLAAGLNLPARRVIVRNYKRYDPNFGMQPIPVLDYKQMAGRAGRPSLDPYGESVLISHTYNEFTDLLDRYIDAEPEDILSKLGTENALRTHVLSTIVNGFATTRQGMVDFMGSSFFAYQQQKWSLIDVVDDCIEFLQDNEMIKDDGERLYATRLGQVISTLYIDPLSGAIIIDKLKKADKVTDMTMLHIICSTPDMRQLYLRSKEYEKINEYVMTHSDEFVEVPNPFKSIEYEWFLGEVKTALLINEWIDEKTLDDITAEFGVGEGDINALSDISEWLMHSAVNLANLTDLDADKAQELEKRIHHGVNKDLIQLVSISNIGRVRARKLYEAGIQSVSDIKNTKLHILSNYLGRKTAYKVLEQLGVEPEDNQQIDEEPESIKSYSGNDQGQKTFNDF

SEQ ID NO: 45 - protein, 747 aa, Methanococcus maripaludis
MHVLDLLKENKITELRPPQKKVIDEGLFDKTKNFLICIPTASGKTLIGEMALLNHILDENKNLTGKKGLFIVPLKALANEKFDEFREKYEKYGIKVGLSIGDFDTKENLSKFHIIITTSEKLDSLMRHNVEWINDVSLAVIDEIHLIGDNERGGTLEVILTKLKNLNAQIVGLSATIGNPEELSNWLNAKLIVDGWRPVELKKGIYFENELEFLKNPAKKIKQVSRNNLTDLIVDSVEEKGSCLIFCNSKRNAVGEAKKHNLAKYLTRTEQHELNKLSEEILSILDRPVETCKALSKCIQNGVAFHHAGLTYKHRKIVEDGFRNRLIKVICCTPTLSAGLNLPCRRAIVRDIKRYSQNGLVDIPRMEIQQCIGRAGRPGLDPYGEGIIYIKNERDAEKAYEILTGSVENIYSKLANQKVLRIHILGLISTGEIKDGQNLVNFMKNTFYAHQFGNIGAVLLNVSEVVEFLEKNKFLETTIHKKTENKVRELSFDSSNNLVLDSKETSFDLTNPNSNIEFRSTKLGKRISELYIDPMSSEIIIEELHELKKKCDQLDRSKIDQYLFYLISKTNEMRPLLRIRPNEELDLILEMDKMGLKDYSIENIEAFKNSKMFCDWVSEIPEEIILEKYGVEPGILRYKVEQAKWMIYSTKEIAKLIHLDNSEIYKSLLKMEVRIEYGAKEELIELLNVKNVGRIRSRKLYDAGIRSKIEINKNPEKILELFGEKIGKKILGEHGMKYGQQTLLNFN

SEQ ID NO: 46 - protein, 799 aa, Natrialba magadii
MNVEELSGLPPGARSHFQEQGIEELYPPQAEAVEAGATEGENLVAAVPTASGKTMIAALSMLSAVQRGGKALYIVPLRALASEKKAEFDAYEEFGVTTGVATGNYESTSEWLATKDIIVATSEKVDSLVRNGADWLSDLTCVVSDEVHLIDDRNRGPTLEVTLAKLRRLNPQLQVVALSATVGNADELADWLDAELVDTDWRPIDLQMGVHYGNALNFDDGETREVPVEAGEKQEAALVRDILQEGGSSLVFVNSRRNAEAAARRLGQVSSRELTAGEQNDLAALATEIREDSDTETSQDLADCVERGAAFHHAGLSSTQRSLVEDAFRDRLLKVISATPTLAAGVNTPARRVIVRDWRRFDPSAGGMAPLDVLEVHQMMGRAGRPGLDPYGEAVLLAKSHDESQELFDRYVWADPEPVRSKLAAEPALRTHVLATIASGFARTREGLLEFLEATLYASQSSEGGRLERVTDDVLSYLERNDFIERSGGPEDTLNSEADAASAFTSAADLADSDGGDSGGTTGQEEDLEATSLGHTVSRLYLDPMSAAEIVHGLEDADERPTALGLYQLVSRTPDMYELYLRSGEDEKFGELYYERERELLGDAPSEFEEERFEDWLAALKTGKLLEDWATEDDEEQITERYKIGPGDLRGKVDTAEWLLGAAESLASEIDSEWAVAVREARARVEHGVGEELLELVSVSGIGRKRARRLYAAGIEEPAALRSADKGVILHVLKGEKTAENILENAGREEPSMDGVEPIPVEGGSGSGSSNSSGSSEPNADANATEDDADDNQSSLGDF

SEQ ID NO: 47 - protein, 723 aa, Methanoregula boonei
MQIQDLAIPEPLRQQYLGLGIRELYPPQAACVERGLLDGKNLLVAIPTASGKTLIAEMAMHRHIANGGKCLYIVPLKALASEKYEEFGNKGVKVGLSTGDLDRRDDALGKNDIIVATSEKVDSLLRNGARWIPDITLVVIDEIHLIDSPDRGPTLEMVIAKMRSKNPGMQLIGLSATIGNPKVLAGWLDAELVTSSWRPVDLRQGVFYDNRIQFAERMRPVKQVSKNYDDLNLCLDTIAEGGQCLVFVSSRRNAEAFAKRAAGAIKSEDAALAACAERLLEGTPTEMVKTLAACVAKGAAFHHAGLSRKERSIVEEAFRKNLLKCISSTPTLAAGLNLPARRVIIRDYLRFSAGEGMQPIPVSEYRQMAGRAGRPRLDPYGEAVLIAKEAEQVPELFEVYIEAEAEDVHSRIAEPTALYTHVLSLVASGFAGTRGELTEFMNRSFYVHEHKQGRLIHRAIDEALQFLITAEMVVEVGEHIGATELGTLVSRMYIDPRSAFAIVTTLREQEKYADLGLIQLICTTPDMPTLYAKNADLPALSRMLEVRGADIWLPPPLDDDAAETYYRAVKTAMLLSDWTDELSEEKICERYGVGPGDVFGMVENINWLLHATSQLARMFVPKFYGQIADCEICMKNGIRRELLPLVRLRGIGRVRARRLFNNGITSPEELSRHKKEDLVKILGSGIAEQVLEQLHPSKDTGKKEPPSGDKNTNPGQSTLFHFG

SEQ ID NO: 48 - protein, 681 aa, unidentified
  source 1..681: note = Ferroplasma acidarmanus
MKLSEITPSEFLKVTDNNDFTLYEHQEEAVAKLRENKNVIVSVPTASGKTLIGYISIYDTYLKGKKSMYIVPLRSLAMEKFSELLSLRNLGVKVTMSIGDYDVPPSFVKNYDVIIATSERADSMLHRDPDILNYFGLVIIDEIHMISDPSRGPRLETVISSLLYLNPEILLLGLSATVSNIQEIAEWMNAETVVSNFRAVPLETGIIFKGNLITDGEKKHLGRDDEVSLIKESIESGGQALVFRNSRRNAEKYAQSMVNFFDFQNDFEKLEIPPDLFNEAQANMVAHGVMFHHAGLSNDQRTMIEKLFKQGYIKILTATPTLAAGVNLPARTVIIRDITRFSDGYSKPISGIEIQQMIGRAGRPKYDKKGYGYIYAASPGMLRVAEGYLTGELEPVISRMDSNSLIRFNVLALISSGIATDLKGIQDFYGKTLLAAQNDIDGYELAFESALYFLKDNDFITEENDIYSATKFGRLTSDLYIDPVSSLILKKCLDLEFSEELYLYYISKTPDMLTFNYRASDYEYLEEFLDRHNISDFSEESMGAAKTAIILNEWINEVPINTIAETFGIGPGDIQAKASSADWISYSLYRLGSMFDKENENNLLHLNIRIKEGVKEEIIRIIEIPQVGRVRGRRLYNNGFKSIDDIANARVEDISRIFGFSTKLAKDIIENAGKLNNRYYR

SEQ ID NO: 49 - protein, 696 aa, Methanocaldococcus fervens
MPTNKILEILKDFGIEELRPPQKKALEKGLLDKNKNFLISIPTASGKTLIGEMALINHLLDENKNPTNKKGIFIVPLKALASEKYEEFKNKYERYGLRVALSIGDYDEDEDLSRYHLIITTAEKLDSLWRHKIDWIDDVSVVVVDEIHLINDESRGGTLEILLTKLKKFNIQIIGLSATIGNPEELANWLNAELIVDDWRPVELKKGIYKNGIIEFINGENREIKAINNNDIYNLVVDCVKDGGCCIVFCNTKRGAVNEAKKLNLKKFLTNEEKRKLKEVAEEILSILEPPTEMCKTLAECILNGSAFHHAGLTYQHRKIVEDAFRNKLIKVICCTPTLSVGLNLPCRRAIVKDLTRYTNRGMRYIPIMEIQQCIGRAGRLGLDPYGEGIIVAKNDRDYLRSYQVLTQKPEPIYSKLSNQAVLRTQLLGLIATIEIRDEYDLEWFIRNTFYAYQYGNLREVAKNINEVIRFLEEKEFMIDFIPTELGKRVAELYIDPLSAKYMIDGLNEMENEDDIYYLYLISKTLEMMPNLRVYKSEELNLIDEMENLGIKSFEIEDLEAFKTAKMLYDWISEVPEDEILKKYKIEPGILRYKVENAVWLMHALKEMAKIIGKNSEIPEKLEIRLEYGAKEDIIELLNVKYIGRVRARKLYNAGIRNVEDIINNPSKVASIIGEKITKKILEDLGIKFGQQKLIF

SEQ ID NO: 50 - protein, 729 aa, Methanocaldococcus jannaschii
MDKILEILKDFGIVELRPPQKKALERGLLDKNKNFLISIPTASGKTLIGEMALINHLLDGNKNPTNKKGIFIVPLKALASEKYEEFKSKYERYGLRIALSIGDYDEDEDLSKYHLIITTAEKLDSLWRHKIDWINDVSVVVVDEIHLINDETRGGTLEILLTKLKEFNVQIIGLSATIGNPDELAEWLNAELIVDDWRPVELKKGIYKNEAIEFINGEIREIKAVDNNDIYNLVVDCVKEGGCCLVFCNTKRNAVNEAKKLNLKKFLTEEEKIRLKEIAEEILSILEPPTEMCKTLAECILNGSAFHHAGLTYQHRKIVEDAFRKRLIKVICCTPTLSAGLNLPCRRAIVKDLTRFTNKGMRYIPIMEIQQCIGRAGRPGLDPYGEGIIVAKNDRDYLRAYQALTQKPEPIYSKLSNQAVLRTQLLGLIATGEIRDEYDLEWFIRNTFYAHQYGNLREVAKNINEVIRFLEENEFIIDFMPTELGKRVSELYIDPLSAKFIIDGLEEMENEEEIYYLYLISKTLEMMPNLRVYNSEELNLIDEMDSLGIKSFEIEDLEAFKTAKMLYDWINEVPEDEILKRYKIEPGILRYKVENAVWIMHALKEIAKLIGKSSDIPEKLEIRLEYGAKEDIIELLSIKYIGRVRARKLYNAGIRSIEDIINNPSKVASIIGEKIAKKILDELGVKFGQQKLSFSGGSAWSHPQFEKGGGSGGGSGGSAWSHPQFEKKL

SEQ ID NO: 51 - protein, 670 aa, Methanocaldococcus infernus
MDEILKFLGIKELRPPQKKALELGILDKKKNFLISIPTGAGKTVIAEMALINHLLLDKGKKGVYIVPLKALASEKYEEFKKKYEKFGVRVALSIGDYDEDEDLENYDLIITTAEKFDSLWRHGIKLSDISVVVVDEIHVIGDSERGGTLEVLLTKLKELDVQIIGLSATIGNPEELSEWLNAELLLDNWRPVELRKGIYREGVIEYLDGEVKECQDIVKEVVKDNGSVIIFCPTKKKAENRALSLDLSDLLKKSEKRKLEEISEELLSLFDPPTELCKKLASCVRKGIAFHHSGLTYEHRKIIEKAFRERILKVICSTTTLAFGLNLPCRRVIISELKRYTRRGLTYIPIMEVQQCIGRAGRPGLDEYGEGILVAKDERDYLRALQCLTQKPEPIYSKLSNDSVLRTQILGLIATRYVLDEYDLEEFIKNTFYAYQYKNLDEIKKKIKEIIEFLEDCNFIKNFEVTPLGKKVSNLYLDPLSAKIMIDNIEVKDDLHLLYILCKCIEMKPLLRVYRKEEEELAEELLNYEIFISYENLEEFKTAKMLYDWINEVPEDEILKTYKVEPGILRYKVEVAKWLSYSLKEIAKILNKEVPNLELRLEYGAKEELLELLKIKYIGRVRARKLYSAGIRNREDIIKNPKKVANILGEKISKKIFEELGVRYGQQRLI

SEQ ID NO: 52 - protein, 799 aa, Methanospirillum hungatei
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 53 - protein, 702 aa, Archaeoglobus fulgidus
MKVEELAESISSYAVGILKEEGIEELFPPQAEAVEKVFSGKNLLLAMPTAAGKTLLAEMAMVREAIKGGKSLYVVPLRALAGEKYESFKKWEKIGLRIGISTGDYESRDEHLGDCDIIVTTSEKADSLIRNRASWIKAVSCLVVDEIHLLDSEKRGATLEILVTKMRRMNKALRVIGLSATAPNVTEIAEWLDADYYVSDWRPVPLVEGVLCEGTLELFDGAFSTSRRVKFEELVEECVAENGGVLVFESTRRGAEKTAVKLSAITAKYVENEGLEKAILEENEGEMSRKLAECVRKGAAFHHAGLLNGQRRVVEDAFRRGNIKVVVATPTLAAGVNLPARRVIVRSLYRFDGYSKRIKVSEYKQMAGRAGRPGMDERGEAIIIVGKRDREIAVKRYIFGEPERITSKLGVETHLRFHSLSIICDGYAKTLEELEDFFADTFFFKQNEISLSYELERVVRQLENWGMVVEDHHLAPTKLGSLVSRLYIDPLTGFIFHDVLSRMELSDIGALHLICRTPDMERLTVRKTDSWVEEEAFRLRKELSYYPSDFSVEYDWFLSEVKTALCLKDWIEEKDEDEICAKYGIAPGDLRRIVETAEWLSNAMNRIAEEVGNTSVSGLTERIKHGVKEELLELVRIRHIGRVRARKLYNAGIRNAEDIVRHREKVASLIGRGIAERVVEGISVKSLNPESAAALEHHHHHH

SEQ ID NO: 54 - protein, 791 aa, Haloterrigena turkmenica
MNLEELTGLPPGATDHFRGEGIEELYPPQADAVEAGATDGENLVAAVPTASGKTMIAALSMLSAVQRGGKALYIVPLRALASEKKEEFEAYEEFGVTTGVTTGNYESTDDWLATKDIIVATSEKVDSLVRNGADWLSELTCVVSDEVHLIDDRNRGPTLEVTLAKLRRLNPGMQVVALSATVGNADEIADWLDASLVDTDWRPIDLQMGVHYGNALNFDDGSTREVPVEGSEKQEAALVRDILREGGSSLVFVNSRRNAEGAAKRLGQVSSREITEDERAELAELADDIRDDSDTETSADLADCVERGAAFHHAGLSSTQRSLVEDAFRDRLLKVISATPTLAAGVNTPARRVIVRDWRRFDPSAGGMAPLDVLEVHQMMGRAGRPGLDPYGEAVLLAKSHDESEELFDRYIWADPEPVRSKLAAEPALRTHVLATIASGFARTRGGLLEFLEATLYASQSSEAGRLESVTDDVLDYLERNDFIERSRDDEAEDSGEDDGPFTSAADLAEQQAAKREETLEATSLGHTVSRLYLDPMSAAEIVHGLERADERPTALGLYQLVSRTPDMYELYLRSGEDEKFGELFYERETELLGDAPSEYEEDRFEDWLAALKTGKLLEDWADETDEETITDRYKIGPGDLRGKVDTAEWLLGAAESLAAEIDSEWTVAVREARARVEHGVGEELLELVSVGGVGRKRARRLYDAGIEEPADLRSADKGIVLSVLKGEKTAENILENAGREDPSMDGVEPADGGPAVGAATNGSSGGSETDETGRADAAESDDSQSSLGDF

SEQ ID NO: 55 - protein, 752 aa, Haladaptatus paucihalophilus
MNVADLTGLPDGVPEHFHAQGIEELYPPQAEAVEAGITEGESVVASIPTASGKTFIAELAMLSSVARGGKALYIVPLRALASEKKEEFEEFEQYGVSIGVSTGNYESDGDWLASRDIIVATSEKVDSLVRNGAKWIDDLSCVVADEVHLVNDAHRGPTLEVTLAKLRRVNPDLQTVALSATVGNAGEMADWLDATLVDSTWRPIDLRKGVLYGQALHFDDGTQQELARGNEKETAALVRDTLEDGGSSLVFVNSRRNAEAAAKRLADVTKTHLTDDERRDLLDIADQIRDVSDTETSDDLATAIEKGAAFHHAGLASDHRSLVEDAFRDKLIKVISATPTLAAGVNTPSRRVIVRDWRRYDGDIGGMQPLDVLEVHQMFGRAGRPGLDPHGEAVLIAKSHDELQELFDQYVWADPEPVHSKLAAEPALRTHILATVASGFAGTEEELLDFLERTLYATQTDETGRLETVTQHVLDYLDRNGFLERDDRLRATGLGHRVSQLYLDPMSAAEIIDGLRDADGKPTALGLYHLVSRTPDMYQLYLRSGDRERYTEIAYEREPEFLGHMPSEFEDNAFEDWLSALKTARLLEDWASELDEDRITERYAIGPGDIRGKVETAQWLLNAAERLAAELQRDDAEGIPSATTTAVREARKRVEYGVEEELLDLAGVRNVGRKRARRLYEAGIESRADLREADKSVVLGALRGRKKTAENILENVGRQDPSLDDVEADAETAATSARATNDGGQQSLGDFE

SEQ ID NO: 56 - protein, 8 aa, synthetic construct
  REGION 1..8: note = Synthetic Polypeptide
QMFGRAGR

SEQ ID NO: 57 - protein, 9 aa, synthetic construct
  REGION 1..9: note = Synthetic Polypeptide
QMFGRAGRP

SEQ ID NO: 58 - protein, 783 aa, unidentified
  source 1..783: note = Halobacterium sp. NRC-1
MRVADVPGLPGGVADHFEGEGVEELYPPQAEAVERGVTEGANLVASVPTASGKTLIAQLAMLSAIAEGGDSPTFSGDGTALYIVPLRALAGEKAQEFEAFERFGLSVGVSTGNYERDGARLADNDIVVATSEKVDSLVRNGAGWIDDLSCVVADEVHLVDDDHRGPTLEVTLAKLRQQVADLQVVALSATVGNAGELAAWLDAELVDSDWRPIELRTGVHYGQSLHYDDGTQAELSVGSGSQTAAVVADTLADDGSTLVFVNSRRNAEASARRLADVTGNALSSAERERLADIAAEIRGVSDTETSDELADAVASGAAFHHAGLAREHRELVEEAFRDRLVKAVSATPTLAAGVNTPARRVVVRDWQRYDGTAGGMQPLDVLEVHQMFGRAGRPGLDPYGEAVLLANSHDELEELFDRYVYADPEPVRSKLAAEPALRTHVLAAIATGFTTTEDGLHEFLGGTLYATQTDDTGRLRSVTGDVLRYLDRNGFVERDGAALRATATGQLVSRLYVDPMSAATIIDGLRDAARDATETDDEGAFRPASELGDDAALPADASVEPTPLGLYHLVSRTPDMYELYLRSGDREQYTEVAYEHEDELLGATPREEQAEFEDWLSALKTARLMADWASELDEERIAERYDVGPGDIRGKVETAEWLLNAAERLAGELDVECGPAVREARKRVQYGVREELLGLAGVRNVGRKRARRLYNAGVESRADLRNADKGVVLGAVRGRAATAERILETVGHPDPGMDGVAADTDAAPESGGEAGGDEGQASLGDFS

SEQ ID NO: 59 - DNA, 450 bp, synthetic construct
  misc_feature 1..450: note = Synthetic Polynucleotide
  source 1..450: mol_type = other DNA
ttttttttttttttttttttttttttttttttttttttttttttttttttggttgtttctgttggtgctgatattgcttttgatgccgaccctaaattttttgcctgtttggttcgctttgagtcttcttcggttccgactaccctcccgactgcctatgatgtttatcctttgaatggtcgccatgatggtggttattataccgtcaaggactgtgtgactattgacgtccttccccgtacgccgggcaataacgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtc

SEQ ID NO: 60 - DNA, 57 bp, synthetic construct
  misc_feature 1..57: note = Synthetic Polynucleotide
  source 1..57: mol_type = other DNA
gcaatatcagcaccaacagaaacaacctttttttttttttttttttttttttttttt

SEQ ID NO: 61 - DNA, 80 bp, synthetic construct
  misc_feature 1..80: note = Synthetic Polynucleotide
  source 1..80: mol_type = other DNA
ctattctgtttatgtttcttgtttgttagccctattctgtccccccccccacccccccccacccccccccaccccccccc

SEQ ID NO: 62 - DNA, 40 bp, synthetic construct
  misc_feature 1..40: note = Synthetic Polynucleotide
  source 1..40: mol_type = other DNA
acagaatagggctaacaaacaagaaacataaacagaatag

SEQ ID NO: 63 - DNA, 60 bp, synthetic construct
  misc_feature 1..60: note = Synthetic Polynucleotide
  source 1..60: mol_type = other DNA
ctattctgtttatgtttcttgtttgttagccctattctgtccccccccccaccccccccc

SEQ ID NO: 64 - DNA, 40 bp, synthetic construct
  misc_feature 1..40: note = Synthetic Polynucleotide
  source 1..40: mol_type = other DNA
ctattctgtttatgtttcttgtttgttagccctattctgt

SEQ ID NO: 65 - DNA, 20 bp, synthetic construct
  misc_feature 1..20: note = Synthetic Polynucleotide
  source 1..20: mol_type = other DNA
ccccccccccaccccccccc

SEQ ID NO: 66 - DNA, 40 bp, synthetic construct; identical to SEQ ID NO: 64
  misc_feature 1..40: note = Synthetic Polynucleotide
  source 1..40: mol_type = other DNA

SEQ ID NO: 67 - DNA, 974 bp, synthetic construct
  misc_feature 1..974: note = Synthetic Polynucleotide
  source 1..974: mol_type = other DNA
ttttttttttttttttttttttttttttttttttttttttttttttttttggttgtttctgttggtgctgatattgctgtgttctatgtcttattctgtgtatgtatcttgtctgttagccccgattgttaccggataattcgagctcggtacccaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccagtattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggatcctctagagtcgacctgcaggcatgcaagctatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattcttgaagacgaaagggcctcgtgatacgcctatttttataggttaatgtcatgataataatggtttcttagacgt

SEQ ID NO: 68 - DNA, 893 bp, synthetic construct
  misc_feature 1..893: note = Synthetic Polynucleotide
  source 1..893: mol_type = other DNA
ctaagaaaccattattatcatgacattaacctataaaaataggcgtatcacgaggccctttcgtcttcaagaattctcatgtttgacagcttatcatcgataagctagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatagcttgcatgcctgcaggtcgactctagaggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatactggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattatccggtaacaatcggggctaacagacaagatacatacacagaataagacatagaacaca

SEQ ID NO: 69 - DNA, 28 bp, synthetic construct
  misc_feature 1..28: note = Synthetic Polynucleotide
  source 1..28: mol_type = other DNA
gcaatatcagcaccaacagaaacaacct

SEQ ID NO: 70 - DNA, 117 bp, synthetic construct
  misc_feature 1..117: note = Synthetic Polynucleotide
  source 1..117: mol_type = other DNA
tcgctgctccacaggtctcagcttgagcagcgaaaataagaacattatgatcagtaggagcactacgacctttgttctggtgctcgtccgggcgcccaaagtggagcgagtgccccc

SEQ ID NO: 71 - DNA, 80 bp, synthetic construct
  misc_feature 1..80: note = Synthetic Polynucleotide
  source 1..80: mol_type = other DNA
gcactcgctccactttgggcgcccggacgagcaccagaacaaaggtcgtagtgctcctactgatcataatgttcttattt

SEQ ID NO: 72 - DNA, 33 bp, synthetic construct
  misc_feature 1..33: note = Synthetic Polynucleotide
  source 1..33: mol_type = other DNA
tcgctgctcaagctgagacctgtggagcagcga

SEQ ID NO: 73 - DNA, 113 bp, synthetic construct
  misc_feature 1..113: note = Synthetic Polynucleotide
  source 1..113: mol_type = other DNA
tcgctgctccacaggtctcagcttgagcagcgaaaataagaacattatgatcagtaggagcactacgacctttgttctggtgctcgtccgggcgcccaaagtggagcgagtgc

SEQ ID NO: 74 - DNA, 28 bp, synthetic construct
  misc_feature 1..28: note = Synthetic Polynucleotide
  source 1..28: mol_type = other DNA
tcgctgctccacaggtctcagcttcccc

SEQ ID NO: 75 - protein, 966 aa, Desulfonatronospira thiodismutans
MPGVDELLQQMGQGDLQGLSTVAVKEIPAREAEFSGIEGLPPPLKQALTESGIENFYTHQARAVNLVRKGRSVVTATPTASGKSLIYNIPVLESIINDPASRALYLFPLKALTRDQLTSLEEFARLLAGKVHVDSAVYDGDTDPQARARIRSKPPNILLTNPDMLHRSFLPYHRSWQKFFSALKYIVVDEVHTYRGVMGSNMAWVFRRLRRICAQYGREPVFIFSSATIANPGQLCSALTGHEPEVIQKGGAPAGKKHFLLLDPEMQGAAQSAIRVLQKALELGLRTIVYTQSRKMTELIAMWASQRAGRLKKYISAYRAGFLPEQRREIEQKLASGELLAVVSTSALELGIDIGHLDLCLLVGYPGSVMATMQRGGRVGRSGRDSAIMLIGHEDALDQYLLRNPREFFSLEPESAVINPDNPSIMRRHLVCAAAEKPIALQEMMLDNEAGKCIKSLEKDGELLASRDRSFYYTRARYPHKDVDLRGTGQTYNIFEHSTGEYLGEVDGVRAFKETHPGAVYLHMGETYVVQDLDLETFAVYAAKSEANYYTRPITEKYTEIVEVQATRATAAGELCLGRLKVTEHVSAYEKRLVRGQARIGLIPLDLPPLVFETQGMWFTLDSQVRRDVEDRRLHFMGGLHALEHGLIGCMPLIILTDRNDLGGIASPVHEQLHKGAVFIYDGTPGGIGLCRQAFELGDRLVARAMGILSSCTCENGCPGCIHSPKCGSGNRPLDKEAAMHMLAVLAGERCGEAKRKDVSCRIETDEGSMEIDSGYTKSDQAELPYAVLDIETRYSAQEVGGWGNCHRMGVSFAVVFDSRNQEFVTFDQEQAADLGSFLEDFSLVVGFNLLKFDYRVLQGLSDYDFSSLPTLDMLREIEARLGHRLSLDHLARHTLGTNKSANGLMALKWWKEGELDKIVEYCRQDVSVTRDLYLFGRDKGYLLFKNKAGKKVRIPVSWQDTAFQV

SEQ ID NO: 76 - protein, 799 aa, Haloarcula marismortui
MDVADLPGVPEWLPDHLRDDGIEELYPPQAEAVEAGVTEGENLVASIPTASGKTLIAELAMLSSVARGGKALYIVPLRALASEKQADFEEFEQYGLDIGVSTGNYESEGGWLADKDIVVATSEKVDSLVRNDAPWIEDLTCVVTDEVHLVDDGERGPTLEVTLAKLRRLNPDLQTVALSATIGNAEALATWLDAGLVDSDWRPIDLQKGVHYGQALHLEDGSQQRLSVQNNEKQTAAIVRDTLEDDGSTLVFVNSRRNAEAAAGRLANTVRPHLSTEERDQLADIAEEIRDVSDTETSDDLADAVADGAAFHHAGLSRGHRELVEDAFRDRLVKVVCATPTLAAGVNTPSRRVVVRDWRRYDGSAGGMAPLSVLEVHQMMGRAGRPGLDPYGEAVLIASSHDEVDELFERYVWADPEPVRSKLAAEPALRTHILATVASGFARSRKGLLEFLEQTLYASQTDDSGQLERVVDDVLTYLQRNDFLEIEAGELDATSLGHTVSRLYLDPMSAAEIVDGLRDWERGASDSTSASGSPADAQAEPPANSGFTTASELAEDADESDADRDPDDISALGLYHLVSRTPDMYQLYLRSGDREEYEMELFEREEELLGPTPSEFEEGRFEDWLSALKTARLLEDWATEVDEATITDRYGVGPGDIRGKVETAQWLLGAAESLASEVDLDAARAISEARIRVEHGVREELVDLAGVRGVGRKRARRLFQAGITDRAQLRDADKAVVLAALRGRRKTAENVLENAGHRDPSMEGVEPAPDVSVDLNDGADGDASAESTANDDQASLGDF

SEQ ID NO: 77 - protein, 711 aa, Natranaerobius thermophilus
MSETFYLLSERMQKKIWEMGWDEFTPVQDKTIPIVMNTNKDVVVSSGTASGKTEAVFLPILSQIEKDATKDLKILYISPLKALINDQFERIIKLCEKSYIPIHRWHGDVNQNKKKQLTKNPAGILQITPESIESLFINRTNELNYILSDIEFIIIDELHAFLDNERGVHLRSLLSRLENYIKEKPRYFALSATLNNFKLIKEWINYNDIKNVEIIDSNEDDKDLLLSLMHFDKGKDYKKPIDLYQDLRELTKNVHSLIFCNSRAEVEETTLYLNRLANREVNTELYLAHHSSIDKKEREYVEKTMANSKSPKSVVTTSSLELGIDIGAIDYVVQIDDTHTVSSLKQRLGRSGRKLGTNQVLQVYSTTNDSLVQSLAVIDLLLEKWIEPATEYPLPLDILFHQIISICHEANGVRLDPLIDNIKANAAFYKLKEEDINHVINYMIENDFLQLIRNSAELIVGLEGERLLRGKEFYAVFMTQEEFEVREGIRKIGSIDKSLMVSEGDNIILAGQLWTIKNIDIERDIIYVAKAVDGKPPKYSGGGFILNPKIPERMHKILCERKNFEFIDNMAQNHLEEQRKPFELYNIKPNERVIWNNGDEILFETYTGTKIFQTLAWILRSYNVNIKEIDGIGRINIEGGIDLPGVLQDIKETDWRPEYLLDFTLEQEKFKSKFSPYLPKDLQDKMHIAHLVDIEGVKTFLENKKIKEIKL

SEQ ID NO: 78 - protein, 491 aa, synthetic construct
  REGION 1..491: note = Synthetic Polypeptide
LPVLEGIELYPPQAEAVEGLLDGKNLLIAIPTASGKTLIAELAMLILGGKALYIVPLRALASEKYEFKFEGVRVGISTGDYDDEWLGDIIVATSEKVDSLLRNWIDITVVVVDEIHLIDRGPTLEVLLAKLRLNPLQIIALSATIGNAEELAEWLAELVVSDWRPVDLRGVFYLFDILVLDTVEGGQLVFNSRRNAEAKKLAVKLTELLAEEIETETSLACVKGAFHHAGLRLVEDAFRLIKVIATPTLAAGLNLPARRVIIRDYKRYGMPIPVLEIQMGRAGRPLDPYGEAVLIAKSDELEYIADPEIWSKLAEALRTHVLALIASGFATELLDFLTFYAYQLIEVLFLNILATLGVSLYIDPLSAIIDGLLGLLHLISTPDMLYLRDELEIEEFFEFLVKTALLDWIEVEDIERYGIGPGDLVEAEWLMHALAKLLLELIRIYGVKEELLELVIRIGRVRARKLYAGIRSDLALILGKIAEKILLGTLF